(19) United States
(12) Patent Application Publication
(10) Pub. No.: US 2025/0388625 A1
(43) Pub. Date: Dec. 25, 2025

(54) CYCLOSPORINE ANALOGUES

(71) Applicant: UCL BUSINESS LTD, London (GB)

(72) Inventors: Greg Towers, London (GB); David Selwood, London (GB); Lucy Thorne, London (GB); Justin Warne, London (GB); Ben Graham, London (GB); Valeria Pingitore, London (GB); Dara Davison, London (GB); Kate Morling, London (GB)

(21) Appl. No.: 18/877,776

(22) PCT Filed: Jun. 20, 2023
(86) PCT No.: PCT/GB2023/051600

§ 371 Date: Dec. 20, 2024

(30) Foreign Application Priority Data
Jun. 21, 2022 (GB) 2209098.9

(51) Int. Cl. C07K 7/64; A61K 35/15; A61P 31/12

(52) U.S. Cl. CPC C07K 7/645; A61K 35/15; A61P 31/12

(57) ABSTRACT

The present application relates to cyclosporine analogues and their use in medical applications.

### FIELD OF THE INVENTION

The present invention relates to novel cyclosporine analogues and their use in medical applications, specifically in gene therapy and as antiviral compounds.

### BACKGROUND TO THE INVENTION

Haematopoietic Stem Cell (HSC) gene therapy can now treat genetic haematopoietic diseases including immunodeficiencies and metabolic disorders that are often otherwise fatal and have limited long-term drug-based therapeutic options. Gene therapy requires HSC isolation and delivery of a functional copy of the disease gene ex vivo. Modified HSC are returned to the patient to replenish the haematopoietic system for long-term therapy.

HSC gene delivery requires vectors based on HIV. A major hurdle is HSC resistance to vector infection. A key protective antiviral protein in HSC, which blocks vector entry and gene delivery, is a known antiviral protein called IFITM3 (see, for instance, Petrillo et al. Cell StemCell 23, 820-832, 2018). As described, for instance, in Petrillo et al. (supra) and WO 2015/162594, the naturally-occurring cyclosporines CsA and CsH have previously been shown to act as transduction enhancers (TE) by inhibiting IFITM3 to enhance vector infection and gene delivery in these cells.

Unfortunately, CsH has limited availability, high cost and poor purity. Conversely, CsA has undesirable features that reduce its efficacy, particularly its inhibition of the well-characterized HIV cofactor cyclophilin A (CypA).

It would be desirable to provide alternative compounds capable of enhancing transduction that do not suffer from these limitations. Particularly desirable would be the provision of easy-to-synthesise, highly potent and selective IFITM3 inhibitors to enhance HIV-vector infection, reduce vector dose required and overcome patient variability.

Certain cyclosporine analogues have been disclosed in WO 2021/229237. However, further developments are needed to the tune the properties of the compounds in relation to obtaining high IFITM3 inhibition, in some cases coupled also with reduced or eliminated CypA inhibition, and hence to obtain compounds that are particularly well suited for use as transduction enhancers in clinical applications.

Meanwhile, the effective treatment of viral infections remains a significant challenge for healthcare systems throughout the world. For instance, there currently exists no known effective treatment for a range of recently emerging coronaviruses, including coronavirus diseases 2019 (“COVID-19”). Cyclosporine compounds have previously been proposed as being potentially useful in the therapy of such conditions (see, for instance, de Wilde et al., J Gen Virol. 2011; 92 (Pt 11): 2542-2548). Meanwhile, more recently it has been proposed that IFITM proteins may promote SARS-Cov-2 infections, for instance by hijacking these usually anti-viral proteins for efficient viral infection, and hence that IFITM proteins may be viable targets for virus inhibition. There remains a pressing demand for efficacious antiviral therapies, with a particularly acute current instance being the need for treatments for COVID-19.

### SUMMARY OF THE INVENTION

A specific class of cyclosporine analogues (more particularly, analogues of CsA) have now been found that are effective as transduction enhancers in HSC gene delivery. It has also been found that the cyclosporine analogues may be effective as antiviral compounds against viruses in which IFITM proteins promote infection, including but not limited to COVID-19.

Specifically, the present invention provides a cyclosporine analogue that is a compound of formula (I):

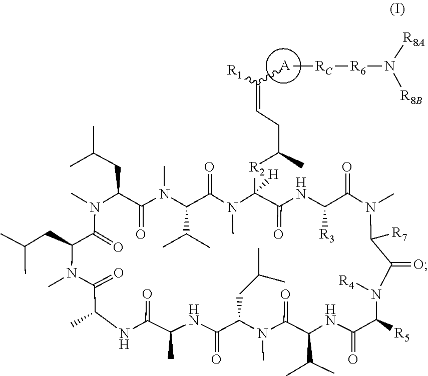

or a pharmaceutically acceptable salt thereof;
wherein:
R₁ represents hydrogen, C₁-C₄ alkyl or C₂-C₄ alkenyl;
R₂ represents

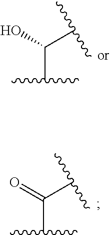

R₃ represents ethyl or isopropyl;
R₄ represents methyl or ethyl;
R₅ represents —CH₂CH(CH₃)₂, —CH₂CH(CH₃)CH₂CH₃, —CH(CH₃)CH₃, or —CH(CH₃)CH₂CH₃;
R₇ represents a hydrogen atom or a moiety that is a C₁₋₂₀ alkyl group, a C₂₋₂₀ alkenyl group or a C₂₋₂₀ alkynyl group, which moiety is unsubstituted or substituted by one or more substituents selected from halogen atoms and sulfonic acid groups, and in which
   (a) 0, 1, 2 or 3 carbon atoms are replaced by groups selected from C₆₋₁₀ arylene, 5- to 10-membered heteroarylene, C₃₋₇ carbocyclylene and 5- to 10-membered heterocyclylene groups, and
   (b) up to half of the —CH₂— groups are replaced by groups selected from —O—, —S—, —C(O)— and —N(C₁₋₆ alkyl)- groups, wherein:
   (i) said arylene, heteroarylene, carbocyclylene and heterocyclylene groups are unsubstituted or substituted by one or more substituents selected from halogen atoms and C₁₋₆ alkyl, C₁₋₆ haloalkyl, C₁₋₆ alkoxy, —(C₁₋₆ alkyl)ₙC(O)O(C₁₋₆ alkyl) (where n=0 or 1), —(C₁₋₆ alkyl)ₙOC(O)(C₁₋₆ alkyl) (where n=0 or 1), C₁₋₆ alkylthiol, —N(R_{N})₂ (wherein each R_{N} independently represents a hydrogen atom or a C₁₋₆ alkyl group), —CN, —S(O)₂NH₂, nitro and sulfonic acid groups; and
   (ii) 0, 1 or 2 carbon atoms in said carbocyclylene and heterocyclylene groups are replaced by —C(O)— groups; and
Ring A represents a monocyclic ring or bicyclic ring system, is a C₆₋₁₀ arylene group or a 5- to 10-membered heteroarylene group, and is unsubstituted or substituted by one or more substituents selected from halogen atoms and C₁₋₆ alkyl, C₁₋₆ haloalkyl, C₁₋₆ alkoxy, —(C₁₋₆ alkyl)ₙC(O)O(C₁₋₆ alkyl) (where n=0 or 1), —(C₁₋₆ alkyl)ₙOC(O)(C₁₋₆ alkyl) (where n=0 or 1), C₁₋₆ alkylthiol, —N(R_{N})₂ (wherein each R_{N} independently represents a hydrogen atom or a C₁₋₆ alkyl group), —CN, —S(O)₂NH₂, nitro and sulfonic acid groups;
and wherein the moiety —R_{C}—R₆—N(R_{8A})(R_{8B}) is defined according to either (X) or (Y): (X).
R_{C} represents a moiety selected from the group consisting of —C(O)O—, —OC(O)—, —C(O)N(R_{N})—, —N(R_{N})C(O)—, —S(O)₂N(R_{N})—, —N(R_{N})S(O)₂—, —N(R_{N})—C(O)—N(R_{N})—, —N(R_{N})—C(S)—N(R_{N})—, —C(O)CH₂—, —CH₂C(O)—, —C(CF₃)N(R_{N})—, —N(R_{N})C(CF₃)—, —C(O)NF—, —NFC(O)—, —C(CN)═N—O—, —O—N—C(CN)—, —N(R_{N})C(O)O—, —OC(O)N(R_{N})—, phenylene, 5- to 6-membered heteroarylene, C₅₋₆ carbocyclylene and 5- to 6-membered heterocyclylene, wherein each R_{N} independently represents a hydrogen atom or a C₁₋₆ alkyl group, and wherein said phenylene, said heteroarylene, said carbocyclylene and said heterocyclylene are each unsubstituted or substituted by one or more substituents selected from halogen atoms and C₁₋₆ alkyl, C₁₋₆ haloalkyl, C₁₋₆ alkoxy, —(C₁₋₆ alkyl)ₙC(O)O(C₁₋₆ alkyl) (where n=0 or 1), —(C₁₋₆ alkyl)ₙOC(O)(C₁₋₆ alkyl) (where n=0 or 1), C₁₋₆ alkylthiol, —N(R_{N})₂ (wherein each R_{N} independently represents a hydrogen atom or a C₁₋₆ alkyl group), —CN, —S(O)₂NH₂, nitro and sulfonic acid groups;
R₆ represents a C₁₋₆ alkylene group, a C₂₋₆ alkenylene group or a C₂₋₆ alkynylene group, and is unsubstituted or substituted by one or more substituents selected from halogen atoms and sulfonic acid groups; and
R_{8A} and R_{8B} either:
   (a) together with the nitrogen atom to which they are attached, form a 5- to 10-membered heteroaryl group or 5- to 10-membered heterocyclyl group, wherein said heteroaryl group and said heterocyclyl group are either unsubstituted or substituted by one or more substituents selected from halogen atoms and C₁₋₆ alkyl, C₁₋₆ haloalkyl, C₁₋₆ alkoxy, —(C₁₋₆ alkyl)ₙC(O)O(C₁₋₆ alkyl) (where n=0 or 1), —(C₁₋₆ alkyl)ₙOC(O)(C₁₋₆ alkyl) (where n=0 or 1), C₁₋₆ alkylthiol, —N(R_{N})₂ (wherein each R_{N} independently represents a hydrogen atom or a C₁₋₆ alkyl group), —CN, —S(O)₂NH₂, nitro and sulfonic acid groups; or
   (b) independently represent a C₁₋₆ alkyl group, a C₂₋₆ alkenyl group or a C₂₋₆ alkynyl group, and are unsubstituted or substituted by one or more substituents selected from halogen atoms, sulfonic acid groups and hydroxy groups;
(Y):
—R_{C}—R₆—N(R_{8A})(R_{8B}) together form a group of formula (VI)

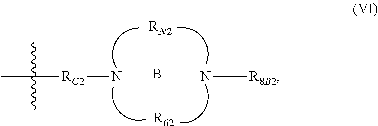

in which
R_{C2} represents —C(O)—, —S(O)₂—, —N(R_{N})—C(O)—, —N(R_{N})—C(S)—, —C(CF₃)— or —OC(O)—, Ring B is a 5-10 membered heterocyclylene ring containing both the nitrogen atom bound to R_{C2} and the nitrogen atom bound to R_{8B2}, and in which R_{N2} and R₆₂ are each alkylene groups, and
R_{8B2} is a C₁₋₆ alkyl group, a C₂₋₆ alkenyl group or a C₂₋₆ alkynyl group, and is unsubstituted or substituted by one or more substituents selected from halogen atoms, sulfonic acid groups, and hydroxy groups.

The present invention further provides use of the cyclosporine analogue of the present invention for increasing the efficiency of transduction of an isolated population of mammalian cells, preferably wherein the mammalian cells are human cells. More preferably, the population of cells is selected from human haematopoietic stem and/or progenitor cells, induced human haematopoietic stem and/or progenitor cells, and/or cells differentiated from the human haematopoietic stem and/or progenitor cells or induced human haematopoietic stem and/or progenitor cells such as cells selected from one or more of a common myeloid progenitor, a megakaryocyte, an erythroblast, a mast cell, a myeloblast, a basophil, a neutrophil, an eosinophil, a monocyte, a common lymphoid progenitor, a natural killer cell, a T cell such as an α/β T cell, a γδ T cell or a regulatory T cell, a B cell and a plasma cell, by a vector derived from HIV-1, HIV-2, SIV, FIV, BIV, EIAV, CAEV or visna lentivirus.

The present invention also provides a method of transducing a population of mammalian cells, preferably wherein the mammalian cells are human cells. More preferably, the population of cells is selected from human haematopoietic stem and/or progenitor cells, induced human haematopoietic stem and/or progenitor cells, and/or cells differentiated from the human haematopoietic stem and/or progenitor cells or induced human haematopoietic stem and/or progenitor cells such as cells selected from one or more of a common myeloid progenitor, a megakaryocyte, an erythroblast, a mast cell, a myeloblast, a basophil, a neutrophil, an eosinophil, a monocyte, a common lymphoid progenitor, a natural killer cell, a T cell such as an α/β T cell, a γδ T cell or a regulatory T cell, a B cell and a plasma cell, comprising the steps of: a) contacting the population of cells with the cyclosporine analogue of the present invention; and b) transducing the population of cells with a vector derived from HIV-1, HIV-2, FIV, BIV, EIAV, CAEV or visna lentivirus.

The present invention also provides a method of gene therapy comprising the steps of: a) transducing a population of mammalian cells, preferably wherein the mammalian cells are human cells. More preferably, the population of cells is selected from human haematopoietic stem and/or progenitor cells, induced human haematopoietic stem and/or progenitor cells, and/or cells differentiated from the human haematopoietic stem and/or progenitor cells or induced human haematopoietic stem and/or progenitor cells such as cells selected from one or more of a common myeloid progenitor, a megakaryocyte, an erythroblast, a mast cell, a myeloblast, a basophil, a neutrophil, an eosinophil, a monocyte, a common lymphoid progenitor, a natural killer cell, a T cell such as an α/β T cell, a γδ T cell or a regulatory T cell, a B cell and a plasma cell, according to the method of transducing of the present invention; and b) administering the transduced cells to a subject.

The present invention further provides a population of mammalian cells, preferably wherein the mammalian cells are human cells. More preferably, the population of cells is selected from human haematopoietic stem and/or progenitor cells, induced human haematopoietic stem and/or progenitor cells, and/or cells differentiated from the human haematopoietic stem and/or progenitor cells or induced human haematopoietic stem and/or progenitor cells such as cells selected from one or more of a common myeloid progenitor, a megakaryocyte, an erythroblast, a mast cell, a myeloblast, a basophil, a neutrophil, an eosinophil, a monocyte, a common lymphoid progenitor, a natural killer cell, a T cell such as an α/β T cell, a γδ T cell or a regulatory T cell, a B cell and a plasma cell, prepared according to the method of transducing of the present invention, as well as a pharmaceutical composition comprising such a population of mammalian cells, human cells, haematopoietic stem and/or progenitor cells, induced haematopoietic stem and/or progenitor cells, and/or cells differentiated from the haematopoietic stem and/or progenitor cells or induced haematopoietic stem and/or progenitor cells such as cells selected from one or more of a common myeloid progenitor, a megakaryocyte, an erythroblast, a mast cell, a myeloblast, a basophil, a neutrophil, an eosinophil, a monocyte, a common lymphoid progenitor, a natural killer cell, a T cell such as an α/β T cell, a γδ T cell or a regulatory T cell, a B cell and a plasma cell. The invention further provides this population of mammalian cells, human cells, haematopoietic stem and/or progenitor cells, induced haematopoietic stem and/or progenitor cells, and/or cells differentiated from the haematopoietic stem and/or progenitor cells or induced haematopoietic stem and/or progenitor cells such as cells selected from one or more of a common myeloid progenitor, a megakaryocyte, an erythroblast, a mast cell, a myeloblast, a basophil, a neutrophil, an eosinophil, a monocyte, a common lymphoid progenitor, a natural killer cell, a T cell such as an α/β T cell, a γδ T cell or a regulatory T cell, a B cell and a plasma cell, for use in therapy.

The present invention still further provides the cyclosporine analogue according to the present invention, for use in mammalian cell, human cell, haematopoietic stem and/or progenitor cell, induced haematopoietic stem and/or progenitor cells, and/or common myeloid progenitor, megakaryocyte, erythroblast, mast cell, myeloblast, basophil, neutrophil, eosinophil, monocyte, common lymphoid progenitor, natural killer cell, T cell such as α/β T cell, γδ T cell or regulatory T cell, B cell and plasma cell, gene therapy.

The present invention further provides cyclosporine analogue according to the present invention, for use in treatment of a pathological condition associated with IFITM3 expression (e.g., a viral infection). Additionally, the present invention provides a method of treating a pathological condition associated with IFITM3 expression (e.g. a viral infection) in a patient in need thereof, which comprises administering to the patient an effective amount of a cyclosporine analogue according to the present invention.

The present invention still further provides a compound of formula

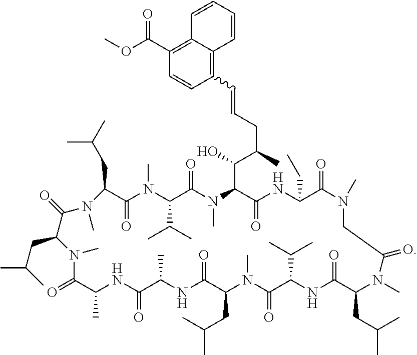 [SMILES: CC[C@@H]1NC(=O)[C@H]([C@H](O)[C@H](C)CC=Cc2ccc(C(=O)OC)c3ccccc23)N(C)C(=O)[C@H](C(C)C)N(C)C(=O)[C@H](CC(C)C)N(C)C(=O)[C@H](CC(C)C)N(C)C(=O)[C@@H](C)NC(=O)[C@H](C)NC(=O)[C@H](CC(C)C)N(C)C(=O)[C@H](C(C)C)NC(=O)[C@H](CC(C)C)N(C)C(=O)CN(C)C1=O]

The present invention still further provides a compound of formula

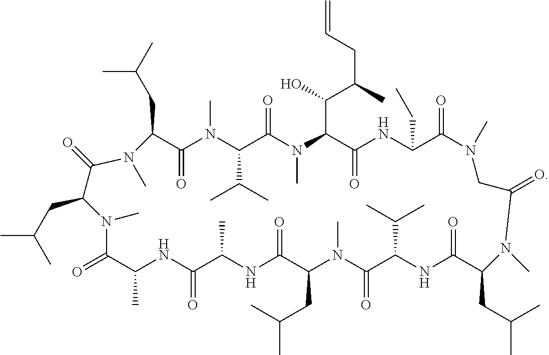 [SMILES: C=CC[C@@H](C)[C@@H](O)[C@H]1C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

### BRIEF DESCRIPTION OF THE DRAWINGS

FIG. 1 shows % infected cells for a range of test compounds at a range of concentrations when tested in THP-1 cells, in the experiments set out in detail in Example 4.

FIG. 2 shows % infected cells for a range of test compounds at a range of concentrations when tested in U87 cells, in the experiments set out in detail in Example 4.

FIG. 3 shows % infected cells for IFN treated (closed symbols) and untreated (open symbols) THP-1 cells when treated with either BG147 (panel A) or CsH (panel B), in the experiments set out in detail in Example 4.

FIG. 4 shows % infected human stem cells in the presence of various transduction enhancers at various concentrations, in the experiments set out in detail in Example 5.

FIG. 5 shows % cell viability of human stem cells in the presence of various transduction enhancers at various concentrations, in the experiments set out in detail in Example 5.

FIG. 6 shows % infected human stem cells in the presence of various transduction enhancers at various concentrations, in further experiments set out in detail in Example 5.

FIG. 7 shows mean fluorescent intensity of human stem cells in the presence of various transduction enhancers at various concentrations, in the experiments set out in detail in Example 6.

FIG. 8 shows the live/dead stain data for the experiments set out in detail in Example 6 for various test compounds at various concentrations.

FIG. 9 shows results from: (a) treating IFITM3 expression-induced THP-1 cells with various compounds, harvesting protein samples and measuring IFITM3 levels by immunoblot, as set out in Example 7 (panel A); and (b) pre-treating Calu-3 lung epithelial cells with either JW3-158 or an equivalent volume of DMSO for 4 h prior to infection with either Alpha, Delta or Omicron isolates of SARS-COV-2 then quantifying the level of viral replication by RT-qPCR, as set out in Example 7 (panel B—for each isolate, left bar shows DMSO study and right bar shows JW3-158 study).

FIG. 10 shows % infected cells for IFNβ-treated THP-1 cells when treated with 1.25, 2.5 or 5 μM various test compounds and GFP vector, in the experiments set out in detail in Example 9 (left, centre, and right bar for each test compound corresponding to 1.25, 2.5 and 5 μM, respectively). For comparison, THP-1 cells that had and had not been subjected to the pre-treatment with IFNβ, were treated with DMSO and are indicated by the bars at the far left of the figure.

FIG. 11 shows the results of Western Blot analysis on THP-1 cells activated by IFNβ and treated with 5 μM of test compounds or DMSO control for 24 hours, in the experiments set out in detail in Example 9.

FIG. 12 shows the results of a MTT colourimetric viability assay of THP-1 cells activated by IFNβ in the presence of test compounds (1.25-5 μM: left, centre, and right bar for each test compound corresponding to 1.25, 2.5 and 5 μM, respectively) or DMSO control, in the experiments set out in detail in Example 9.

### DETAILED DESCRIPTION OF THE INVENTION

### Definitions

As used herein, the term “alkyl” includes both saturated straight chain and branched alkyl groups (i.e., as a monovalent group derived by removing one hydrogen atom from an alkane). Preferably, unless otherwise specified an alkyl group is a C₁₋₂₀ alkyl group, more preferably a C₁₋₁₅, more preferably still a C₁₋₁₂ alkyl group, more preferably still, a C₁₋₆ alkyl group, and most preferably a C₁₋₄ alkyl group. Unless otherwise specified, particularly preferred alkyl groups include, for example, methyl, ethyl, propyl, isopropyl, butyl, isobutyl, tert-butyl, pentyl and hexyl. The term “alkylene” should be construed accordingly (i.e., as the divalent counterpart to an alkyl group, in other words an alkane from which two hydrogen atoms have been removed).

As used herein, the term “alkenyl” refers to a group containing one or more carbon-carbon double bonds, which may be branched or unbranched (i.e., as a monovalent group derived by removing one hydrogen atom from an alkene). Preferably, unless otherwise specified the alkenyl group is a C₂₋₂₀ alkenyl group, more preferably a C₂₋₁₅ alkenyl group, more preferably still a C₂₋₁₂ alkenyl group, or preferably a C₂₋₆ alkenyl group, and most preferably a C₂₋₄ alkenyl group. The term “alkenylene” should be construed accordingly (i.e., as the divalent counterpart to an alkenyl group, in other words an alkene from which two hydrogen atoms have been removed).

As used herein, the term “alkynyl” refers to a carbon chain containing one or more triple bonds, which may be branched or unbranched (i.e., as a monovalent group derived by removing one hydrogen atom from an alkyne). Preferably, unless otherwise specified the alkynyl group is a C₂₋₂₀ alkynyl group, more preferably a C₂₋₁₅ alkynyl group, more preferably still a C₂₋₁₂ alkynyl group, or preferably a C₂₋₆ alkynyl group and most preferably a C₂₋₄ alkynyl group. The term “alkynylene” should be construed accordingly (i.e., as the divalent counterpart to an alkynyl group, in other words an alkyne from which two hydrogen atoms have been removed).

Unless otherwise specified, an alkyl, alkenyl or alkynyl group is typically unsubstituted. However, where such a group (or the divalent counterparts thereto) is indicated to be unsubstituted or substituted (and again unless otherwise specified), one or more hydrogen atoms are optionally replaced by halogen atoms or sulfonic acid groups. Preferably, a substituted alkyl, alkenyl or alkynyl group has from 1 to 10 substituents, more preferably 1 to 5 substituents, more preferably still 1, 2 or 3 substituents and most preferably 1 or 2 substituents, for example 1 substituent. Preferably a substituted alkyl, alkenyl or alkynyl group carries not more than 2 sulfonic acid substituents. Halogen atoms are preferred substituents. Preferably, though, an alkyl, alkenyl or alkynyl group is unsubstituted. A haloalkyl group means an alkyl group that is substituted by one or more halogen atoms.

Where an alkyl, alkenyl or alkynyl group (or the divalent counterparts thereto) is indicated to be unsubstituted or substituted by one or more substituents selected from halogen atoms, sulfonic acid groups, and hydroxy groups (e.g., for each of R_{8A}, R_{8B} and R_{8B2}), then one or more hydrogen atoms are optionally replaced by halogen atoms, sulfonic acid groups, and/or hydroxy groups. Preferably, such a substituted alkyl, alkenyl or alkynyl group has from 1 to 10 substituents, more preferably 1 to 5 substituents, more preferably still 1, 2 or 3 substituents and most preferably 1 or 2 substituents, for example 1 substituent. Preferably such a substituted alkyl, alkenyl or alkynyl group carries not more than 2 sulfonic acid substituents. Halogen atoms and hydroxy groups are preferred substituents. Halogen atoms are preferred substituents. Hydroxy groups are also preferred substituents. Preferably, though, an alkyl, alkenyl or alkynyl group is unsubstituted.

A haloalkyl group means an alkyl group that is substituted by one or more halogen atoms.

As used herein, halogen atoms are typically F, Cl, Br or I atoms.

As used herein, a C₆₋₁₀ aryl group is a monocyclic or polycyclic 6- to 10-membered aromatic hydrocarbon ring system having from 6 to 10 carbon atoms. Unless otherwise specified, phenyl and naphthyl are preferred. The term “arylene” should be construed accordingly (i.e., as the divalent counterpart to an aryl group), with phenylene and naphthylene hence being preferred arylenes unless otherwise specified.

As used herein, a 5- to 10-membered heteroaryl group is a monocyclic or polycyclic 5- to 10-membered aromatic ring system, such as a 5- to 6-membered ring or a a 9- to 10-membered ring system, containing at least one heteroatom, for example 1, 2, 3 or 4 heteroatoms, selected from O, S and N. When the ring or ring system contains 4 heteroatoms these are preferably all nitrogen atoms. The term “heteroarylene” should be construed accordingly.

Examples of monocyclic heteroaryl groups include thienyl, furyl, pyrrolyl, imidazolyl, thiazolyl, isothiazolyl, pyrazolyl, oxazolyl, isoxazolyl, triazolyl, thiadiazolyl, oxadiazolyl, pyridinyl, pyridazinyl, pyrimidinyl, pyrazinyl, triazinyl and tetrazolyl groups.

Examples of polycyclic heteroaryl groups include quinolinyl, isoquinolinyl, indolinyl, benzothienyl, benzofuryl, benzimidazolyl, benzothiazolyl, benzisothiazolyl, benzoxazolyl, benzisoxazolyl, benztriazolyl, indolyl, isoindolyl and indazolyl groups.

As used herein, a 5- to 10-membered heterocyclyl group is a non-aromatic, saturated or unsaturated, monocyclic or polycyclic C₅₋₁₀ carbocyclic ring system in which one or more, for example 1, 2, 3 or 4, of the carbon atoms are replaced with a moiety selected from N, O, S, S(O) and S(O)₂. Preferably, unless otherwise specified the 5- to 10-membered heterocyclyl group is a 5- to 6-membered ring. The term “heterocyclyene” should be construed accordingly.

Examples of heterocyclyl groups include azetidinyl, oxetanyl, thietanyl, pyrrolidinyl, imidazolidinyl, oxazolidinyl, isoxazolidinyl, thiazolidinyl, isothiazolidinyl, tetrahydrofuranyl, tetrahydrothienyl, tetrahydropyranyl, tetrahydrothiopyranyl, dithiolanyl, dioxolanyl, pyrazolidinyl, piperidinyl, piperazinyl, hexahydropyrimidinyl, methylenedioxyphenyl, ethylenedioxyphenyl, thiomorpholinyl, S-oxo-thiomorpholinyl, S,S-dioxo-thiomorpholinyl, morpholinyl, 1,3-dioxolanyl, 1,4-dioxolanyl, trioxolanyl, trithianyl, imidazolinyl, pyranyl, pyrazolyl, thioxolanyl, thioxothiazolidinyl, 1H-pyrazol-5-(4H)-onyl, 1,3,4-thiadiazol-2 (3H)-thionyl, oxopyrrolidinyl, oxothiazolidinyl, oxopyrazolidinyl, succinimido and maleimido groups and moieties.

For the avoidance of doubt, although the above definitions of heteroaryl and heterocyclyl groups refer to an “N” moiety which can be present in the ring, as will be evident to a skilled chemist the N atom will be protonated (or will carry a substituent as defined below) if it is attached to each of the adjacent ring atoms via a single bond.

As used herein, a C₃₋₇ carbocyclyl group is a non-aromatic saturated or unsaturated hydrocarbon ring having from 3 to 7 carbon atoms. Preferably, unless otherwise specified it is a saturated or mono-unsaturated hydrocarbon ring (i.e. a cycloalkyl moiety or a cycloalkenyl moiety) having from 3 to 7 carbon atoms, more preferably having from 5 to 6 carbon atoms. Examples include cyclopropyl, cyclobutyl, cyclopentyl and cyclohexyl and their mono-unsaturated variants. Particularly preferred carbocyclic groups, unless otherwise specified, are cyclopentyl and cyclohexyl. The term “carbocyclylene” should be construed accordingly.

Where specified, 0, 1 or 2 carbon atoms in a carbocyclyl or heterocyclyl group may be replaced by —C(O)— groups. As used herein, the “carbon atoms” being replaced are understood to include the hydrogen atoms to which they are attached. When 1 or 2 carbon atoms are replaced, preferably two such carbon atoms are replaced. Preferred such carbocyclyl groups include a benzoquinone group and preferred such heterocyclyl groups include succinimido and maleimido groups.

Unless otherwise specified, an aryl, heteroaryl, carbocyclyl or heterocyclyl group is typically unsubstituted. However, where such a group is indicated to be unsubstituted or substituted, one or more hydrogen atoms are optionally replaced by halogen atoms or C₁₋₆ alkyl, C₁₋₆ haloalkyl, C₁₋₆ alkoxy, —(C₁₋₆ alkyl) nC(O)O(C₁₋₆ alkyl) (where n=0 or 1), —(C₁₋₆ alkyl)ₙOC(O)(C₁₋₆ alkyl) (where n=0 or 1), C₁₋₆ alkylthiol, —N(R_{N})₂ (wherein each R_{N} independently represents a hydrogen atom or a C₁₋₆ alkyl group), —CN, —S(O)₂NH₂, nitro or sulfonic acid groups. Preferably, a substituted aryl, heteroaryl, carbocyclyl or heterocyclyl group has from 1 to 4 substituents, more preferably 1 to 2 substituents and most preferably 1 substituent. Preferably a substituted aryl, heteroaryl, carbocyclyl or heterocyclyl group carries not more than 2 nitro substituents and not more than 2 sulfonic acid substituents.

As used herein, a alkoxy group is an alkyl (e.g. a C₁₋₆ alkyl or C₁₋₄ alkyl) group which is attached to an oxygen atom.

As used herein, an alkylthiol group is an alkyl (e.g. a C₁₋₆ alkyl or C₁₋₄ alkyl) group which is attached to a sulfur atom.

In some instances, the compounds of the present invention can be provided in the form of a pharmaceutical salt. Substantially any pharmaceutically acceptable salt can be used. Those skilled in the art of preparing compounds for use in medical applications will be familiar with suitable such salt compounds. For instance, the present compounds may be in the form of a salt with a pharmaceutically acceptable base. Pharmaceutically acceptable bases include, but are by no means limited to, alkali metal (e.g. sodium or potassium) and alkali earth metal (e.g. calcium or magnesium) hydroxides and organic bases such as alkyl amines, aralkyl amines or heterocyclic amines.

### Cyclosporine Analogues

Non-limiting, preferred embodiments of the cyclosporine analogues of the present invention are set out below.

R₁ preferably represents hydrogen.

R₂ preferably represents

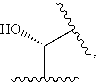

R₃ preferably represents ethyl.

R₄ preferably represents methyl.

R₅ preferably represents —CH₂CH(CH₃)₂.

In R₇, the hydrogen or moiety that is a C₁₋₂₀ alkyl group, a C₂₋₂₀ alkenyl group or a C₂₋₂₀ alkynyl group is preferably hydrogen or a moiety that is a C₁₋₁₅ alkyl group, a C₂₋₁₅ alkenyl group or a C₂₋₁₅ alkynyl group, more preferably hydrogen or a moiety that is a C₁₋₁₂ alkyl group, a C₂₋₁₂ alkenyl group or a C₂₋₁₂ alkynyl group, more preferably still hydrogen or a moiety that is a C₁₋₁₀ alkyl group or a C₂₋₁₀ alkenyl group, and most preferably hydrogen. In these preferred embodiments, preferred options for substituents and for (a) and (b) are as follows.

With respect to substituents, preferably the moiety is unsubstituted or substituted by one or more substituents selected from halogen atoms, and more preferably the moiety is unsubstituted. The maximum number of substituents is preferably 5, more preferably 3.

With respect to (a), preferably 0, 1 or 2 carbon atoms are replaced, and more preferably 0 or 1 carbon atoms are replaced. Such carbon atoms are preferably replaced by groups selected from phenylene, 5- to 6-membered heteroarylene, C₅₋₆ carbocyclylene and 5- to 6-membered heterocyclylene groups, and more preferably by phenylene.

With respect to (b), preferably 0 to 4 —CH₂— groups are replaced, more preferably 0 to 3 groups and most preferably 0, 1 or 2 groups. For any —CH₂— groups that are replaced, preferably they are replaced by groups selected from —O—, —S— and —C(O)— and more preferably by groups selected from —O— and —C(O)—. For the avoidance of doubt, it is acceptable to replace adjacent —CH₂— where chemically meaningful, e.g. to replace —CH₂—CH₂— with —C(O)—O—, —O—C(O)—, etc. Similarly, it is acceptable to replace the —CH₂— of a terminal methyl group (i.e., CH₃ or —CH₂—H), e.g. replace —CH₃ with —OH. Preferably not more than two adjacent —CH₂— groups are replaced (e.g., typically any replacements do not involve replacement of three or more adjacent/contiguous —CH₂— groups).

Preferably any arylene, heteroarylene, carbocyclylene and heterocyclylene groups in (a) are unsubstituted or substituted by one or more substituents selected from halogen atoms and sulfonic acid groups and more preferably they are unsubstituted. Preferably 0 carbon atoms in any carbocyclylene and heterocyclylene groups are replaced by —C(O)— groups.

Preferably R₇ represents hydrogen or a moiety that is a C₁₋₁₅ alkyl group, a C₂₋₁₅ alkenyl group or a C₂₋₁₅ alkynyl group, which moiety is unsubstituted or substituted by one or more halogen atoms, and in which
(a) 0, 1 or 2 carbon atoms are replaced by groups selected from C₆₋₁₀ arylene, 5- to 10-membered heteroarylene, C₃₋₇ carbocyclylene and 5- to 10-membered heterocyclylene groups, and
(b) 0 to 4 of the —CH₂— groups are replaced by groups selected from —O—, —S—, —C(O)— and —N(C₁₋₆ alkyl)-groups, wherein:
(i) said arylene, heteroarylene, carbocyclylene and heterocyclylene groups are unsubstituted or substituted by one or more substituents selected from halogen atoms and sulfonic acid groups; and
(ii) 0, 1 or 2 carbon atoms in said carbocyclylene and heterocyclylene groups are replaced by —C(O)— groups.

More preferably, R₇ represents hydrogen or a moiety that is a C₁₋₁₂ alkyl group, a C₂₋₁₂ alkenyl group or a C₂₋₁₂ alkynyl group, which moiety is unsubstituted or substituted by one or more halogen atoms, and in which
(a) 0, 1 or 2 carbon atoms are replaced by groups selected from phenylene, 5- to 6-membered heteroarylene, C₅₋₆ carbocyclylene and 5- to 6-membered heterocyclylene groups, and
(b) 0 to 3 of the —CH₂— groups are replaced by groups selected from —O—, —S— and —C(O)—;
wherein said phenylene, heteroarylene, carbocyclylene and heterocyclylene groups are unsubstituted or substituted by one or more halogen atoms.

R₇ more preferably still represents hydrogen or a moiety that is C₁₋₁₀ alkyl group or a C₂₋₁₀, in which (a) 0 or 1 carbon atoms are replaced by a phenylene group, and (b) 0, 1 or 2 of the —CH₂— groups are replaced by groups selected from —O— and —C(O)— groups.

R₇ most preferably represents hydrogen.

In one particularly preferred embodiment, R¹ represents hydrogen, R₂ represents

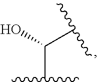

R₃ represents ethyl, R₄ represents methyl, R₅ represents —CH₂CH(CH₃)₂, and R₇ represents hydrogen. It will be appreciated that in this embodiment, the structure of the resulting cyclosporine analogue closely corresponds to that of cyclosporine A (“CsA”), but where the moiety -[Ring A]—R_{C}—R₆—N(R_{8A})(R_{8B}) replaces a methyl group, —CH₃, that is present in the same location in CsA.

The stereochemistry at the ethenyl group that connects the cyclic cyclosporine A core to the groups R₁ and Ring A can be either E or Z. Thus, the chemical formula (I) embraces both chemical formula (Ia) and (Ib):

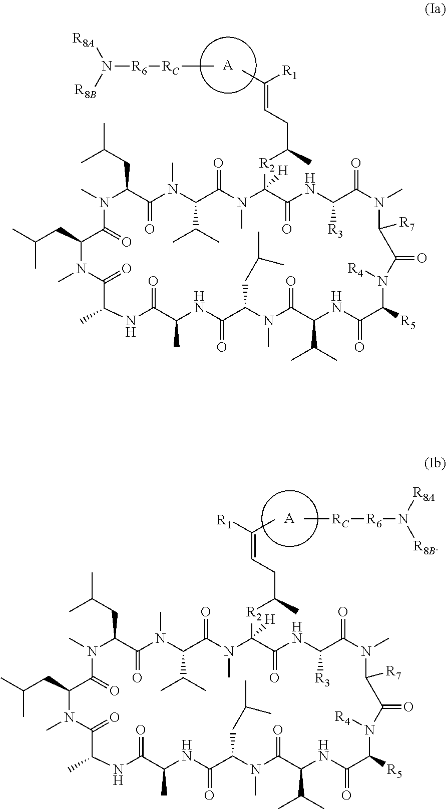

Moieties of formula (IIa) are currently preferred, and most particularly those in which R₁ represents hydrogen, R₂ represents

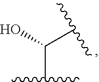

R₃ represents ethyl, R₄ represents methyl, R₅ represents —CH₂CH(CH₃)₂, and R₇ represents hydrogen.

For avoidance of doubt, and regardless of the stereochemistry of the compound (e.g., (IIa) or (IIb) above), where specific definitions are provided herein for any of the divalent moieties Ring A, R_{C} and R₆, using notation of the general form “-[definition]-”, the leftmost portion of the definition is that closest to the central cyclosporine ring and the rightmost portion of the definition is that closest to the terminal —N(R_{8A})(R_{8B}) group. For instance, for a definition of R_{C} as “-a-b-c-”, the group “a” attaches to Ring A and the group “c” attaches to R₆.

In the cyclosporine analogue of the present disclosure, the Ring A replaces the methyl group that is present in cyclosporine A (“CsA”) and, in its place, links the ethenyl group extending from the cyclic core of CsA to a side chain of structure —R_{C}—R₆—N(R_{8A})(R_{8B}).

For avoidance of doubt, while illustrated using a schematic circle in formulas (I), (Ia) and (Ib), and identified for brevity herein in using the singular language “Ring”, the Ring A represents either a monocyclic ring or a bicyclic ring system. A bicyclic ring system in general can be any of fused, bridged and spirocyclic, but most typically when Ring A is a bicyclic ring system, it is a fused bicyclic ring (i.e., it comprises a first ring and a second ring, which share two adjacent ring atoms).

In a first, and often more preferred, aspect, Ring A is an unsubstituted or substituted (fused) bicyclic ring system, such that the ring system has the general formula (IIa), (IIb) or (IIc)

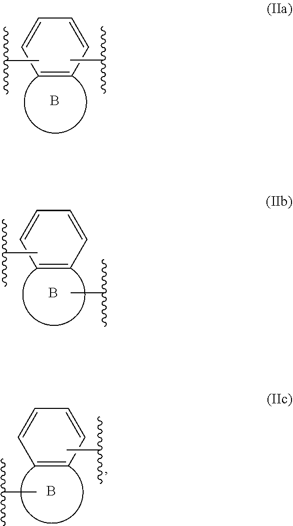

where B is a monocyclic, 5- to 6-membered ring (including the two carbon atoms of the benzene ring to which it is fused). The ring atoms of B may be exclusively carbon atoms, or may contain one or more heteroatoms selected from N, S and O (preferably up to two heteroatoms, and preferably wherein the heteroatoms are N atoms).

Exemplary systems of general formula (IIa), (IIb), and (IIc) include (a) a naphthylene group and (b) a 9- to 10-membered heteroarylene group, such as, but not limited to, a quinolinylene, isoquinolinylene or indolinylene group. Especially preferred is a naphthylene group, for instance of formula (IIIa), (IIIb) or (IIIc):

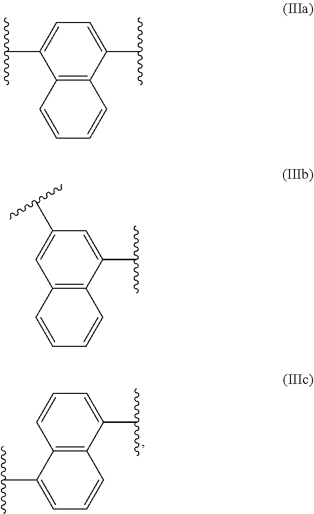 [SMILES: *c1ccc(*)c2ccccc12 ; *c1cc(*)c2ccccc2c1 ; *c1cccc2c(*)cccc12]

and especially preferably of formula (IIIa).

Specific examples of quinolinylene and indolinylene groups include formula (IVa), (IV) and (IVc)

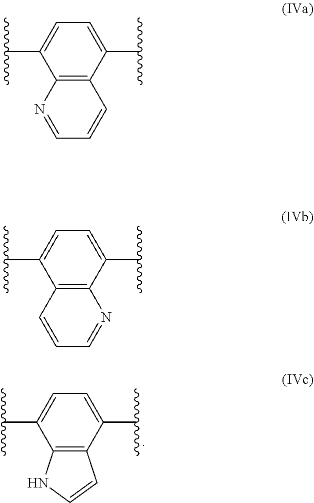 [SMILES: *c1ccc(*)c2ncccc12 ; *c1ccc(*)c2ncccc12 ; *c1ccc(*)c2[nH]ccc12]

For avoidance of doubt, in all of formulae (Ila), (IIb), (IIc), (IIIa), (IIIb), (IIIc), (IVa), (IVb) and (IVc), the connection shown to the right is to R_{C}. Also for avoidance of doubt, in all of formulae (IIa), (IIb), (IIc), (IIIa), (IIIb), (IIIc), (IVa), (IVb) and (IVc), the ring systems are either unsubstituted or substituted (namely, by the said one or more substituents selected from halogen atoms and C₁₋₆ alkyl, C₁₋₆ haloalkyl, C₁₋₆ alkoxy, —(C₁₋₆ alkyl)ₙC(O)O(C₁₋₆ alkyl) (where n=0 or 1), —(C₁₋₆ alkyl)ₙOC(O)(C₁₋₆ alkyl) (where n=0 or 1), C₁₋₆ alkylthiol, —N(R_{N})₂ (wherein each R_{N} independently represents a hydrogen atom or a C₁₋₆ alkyl group), —CN, —S(O)₂NH₂, nitro and sulfonic acid groups). In these ring systems, preferred substituents are selected from C₁₋₄ alkyl, C₁₋₄ haloalkyl and C₁₋₄ alkoxy groups, more preferably C₁₋₄ alkyl groups. Preferably these ring systems are either unsubstituted or substituted by one or two substituents, for instance they are unsubstituted. In a second aspect, Ring A is an unsubstituted or substituted monocyclic ring in which the monocyclic ring is typically a phenylene ring or a 5- to 6-membered heteroarylene group. In this aspect, the monocyclic ring is preferably a phenylene ring and is particularly preferably of formula (V)

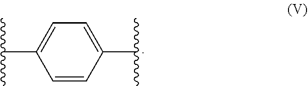 [SMILES: *c1ccc(*)cc1]

For avoidance of doubt, such a phenylene ring or 5- to 6-membered heteroarylene group can be unsubstituted or substituted (namely, by the said one or more substituents selected from halogen atoms and C₁₋₆ alkyl, C₁₋₆ haloalkyl, C₁₋₆ alkoxy, —(C₁₋₆ alkyl) nC(O)O(C₁₋₆ alkyl) (where n=0 or 1), —(C₁₋₆ alkyl)ₙOC(O)(C₁₋₆ alkyl) (where n=0 or 1), C₁₋₆ alkylthiol, —N(R_{N})₂ (wherein each R_{N} independently represents a hydrogen atom or a C₁₋₆ alkyl group), —CN, —S(O)₂NH₂, nitro and sulfonic acid groups).

In this second aspect, Ring A is most preferably a phenylene group of formula (Va)

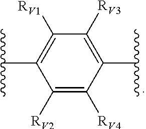

wherein R_{V1}, R_{V2}, R_{V3} and R_{V4} are independently selected from hydrogen and halogen atoms and C₁₋₆ alkyl, C₁₋₆ haloalkyl, C₁₋₆ alkoxy, —(C₁₋₆ alkyl)ₙC(O)O(C₁₋₆ alkyl) (where n=0 or 1), —(C₁₋₆ alkyl)ₙOC(O)(C₁₋₆ alkyl) (where n=0 or 1), C₁₋₆ alkylthiol, —N(R_{N})₂ (wherein each R_{N} independently represents a hydrogen atom or a C₁₋₆ alkyl group), —CN, —S(O)₂NH₂, nitro and sulfonic acid groups.

Preferably in formula (Va), at least two of R_{V1}, R_{V2}, R_{V3} and R_{V4} are hydrogen, and more preferably at least R_{V3} and R_{V4} are hydrogen. Preferably when (at least) one of R_{V1}, R_{V2}, R_{V3} and R_{V4} is other than hydrogen, then it is (at least) R_{V1} that is other hydrogen. Preferably when two of R_{V1}, R_{V2}, R_{V3} and R_{V4} are other than hydrogen, then it is R_{V1} and R_{V2} that are other hydrogen. Particularly preferred non-hydrogen R_{V1}, R_{V2}, R_{V3} and R_{V4} groups are C₁₋₆ alkyl, C₁₋₆ haloalkyl, C₁₋₆ alkoxy, —CN, and S(O)₂NH₂, more preferred being C₁₋₄ alkyl, C₁₋₄ haloalkyl and C₁₋₄ alkoxy groups (e.g., methyl, ethyl, propyl, methoxy, ethoxy, propoxy (including -OiPr), and trifluoromethyl) and most preferred being C₁₋₄ alkyl groups (e.g, methyl, ethyl, and propyl).

In general (i.e., referring to both the first aspect and second aspect discussed herein), in Ring A, it is preferred that said C₆₋₁₀ arylene group or 5- to 10-membered heteroarylene group is a naphthylene group, a 9- to 10-membered heteroarylene group or a phenylene group. Furthermore, it is particularly preferred that, in ring A, said C₆₋₁₀ arylene group or 5- to 10-membered heteroarylene group is a naphthylene group.

Also in general, preferred substituents of ring A, when ring A is substituted, are selected from C₁₋₄ alkyl, C₁₋₄ haloalkyl and C₁₋₄ alkoxy groups, most preferably C₁₋₄ alkyl groups. Preferably, Ring A is unsubstituted or substituted by one or two substituents.

Compounds in which —R_{C}—R₆—N(R_{8A})(R_{8B}) is defined according to (X):

R_{C} is a connector group that connects Ring A to the group R₆.

In R_{C}, any instances of R_{N} are preferably a hydrogen atom or a C₁₋₄ alkyl group, more preferably a hydrogen atom or a methyl group and most preferably a hydrogen atom.

R_{C} preferably represents a group selected from the group consisting of —C(O)O—, —C(O)N(R_{N})—, —S(O)₂N(R_{N})—, —N(R_{N})—C(O)—N(R_{N})—, —N(R_{N})—C(S)—N(R_{N})—, —C(O)CH₂—, —C(CF₃)N(R_{N})—, —C(O)NF—, —C(CN)═N—O—, —N(R_{N})C(O)O— and 5- to 6-membered heteroarylene.

When R_{C} is 5- to 6-membered heteroarylene, preferred such groups are triazolylene (i.e., a divalent group obtainable by removing two hydrogen atoms from triazole), oxazolylene (i.e., a divalent group obtainable by removing two hydrogen atoms from oxazole), isoxazolylene (i.e., a divalent group obtainable by removing two hydrogen atoms from isoxazole) and oxadiazolylene (i.e., a divalent group obtainable by removing two hydrogen atoms from oxadiazole). Preferred instances of triazolylene are 1,2,3-triazolylene, such as

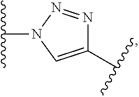 [SMILES: *c1cn(*)nn1]

where preferably the connection shown to the left is to Ring A and the connection to the right is to R₆, and 1,2,4-triazolylene, such as

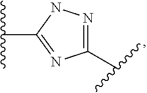 [SMILES: *c1n[nH]c(*)n1]

where preferably the connection shown to the left is to Ring A and the connection to the right is to R₆. Preferred instances of oxazolylene are

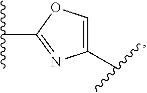 [SMILES: *c1coc(*)n1]

where preferably the connection shown to the left is to Ring A and the connection to the right is to R₆. Preferred instances of oxadiazolylene are 1,2,4-oxadiazolylene, such as

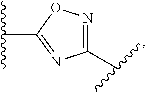 [SMILES: *c1noc(*)n1]

where preferably the connection shown to the left is to Ring A and the connection to the right is to R₆.

R_{C} more preferably represents a group selected from the group consisting of —C(O)O—, —C(O)N(R_{N})— (especially —C(O) NH—), —S(O)₂N(R_{N})— (especially —S(O)₂NH—), —N(R_{N})—C(O)—N(R_{N})— (especially —NH—C(O)—NH—), —N(R_{N})—C(S)—N(R_{N})— (especially —NH—C(S)—NH—), —N(R_{N})C(O)O— (especially —NH—C(O)—O—) and triazolylene (especially 1,2,3-triazolylene such as indicated above).

Hence, more preferred R_{C} groups are —C(O)O—, —C(O) NH—, —S(O)₂NH—, —NH—C(O)—NH—, —NH—C(S)—NH—, —NH—C(O)—O— and

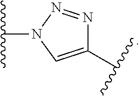 [SMILES: *c1cn(*)nn1]

Most preferably R_{C} is —C(O)O—.

R₆ is a linker group that connects R_{C} to the moiety-N(R_{8A})(R_{8B}).

R₆ is preferably either unsubstituted or substituted by one or more (e.g. up to four) halogen atoms. Most preferably R₆ is unsubstituted.

Preferably R₆ represents a C₂₋₄ alkylene group or a C₂₋₄ alkenylene group, and more preferably a C₂₋₄ alkylene group, and more preferably still —(CH₂)₂— or —(CH₂)₃— or —(CH₂)₄—.

Most preferably R₆ is ethylene, i.e. —(CH₂)₂—. Another especially preferred R₆ is (CH₂)₄—.

The groups R_{8A} and R_{8B} are attached to a nitrogen atom and together form either a heterocyclic group with this nitrogen atom or a tertiary amine group.

In a first aspect of the invention, R_{8A} and R_{8B}, together with the nitrogen atom to which they are attached, form a 5- to 10-membered heteroaryl group or 5- to 10-membered heterocyclyl group, wherein said heteroaryl group and said heterocyclyl group are either unsubstituted or substituted by one or more substituents selected from halogen atoms and C₁₋₆ alkyl, C₁₋₆ haloalkyl, C₁₋₆ alkoxy, —(C₁₋₆ alkyl)ₙC(O)O(C₁₋₆ alkyl) (where n=0 or 1), —(C₁₋₆ alkyl)ₙOC(O)(C₁₋₆ alkyl) (where n=0 or 1), C₁₋₆ alkylthiol, —N(R_{N})₂ (wherein each R_{N} independently represents a hydrogen atom or a C₁₋₆ alkyl group), —CN, —S(O)₂NH₂, nitro and sulfonic acid groups. Preferably said heteroaryl group and said heterocyclyl group are unsubstituted. However, in certain embodiments it may be desirable for said heteroaryl group and said heterocyclyl to comprise one or more (e.g. one) amino substituents (e.g., —NH₂), for instance so as to achieve a physiological pKa (e.g., between 7.5 and 10).

In a second aspect of the invention, R_{8A} and R_{8B} independently represent a C₁₋₆ alkyl group, a C₂₋₆ alkenyl group or a C₂₋₆ alkynyl group, and are unsubstituted or substituted by one or more substituents selected from halogen atoms, sulfonic acid groups and hydroxy groups. For instance, in this second aspect of the invention, R_{8A} and R_{8B} independently represent a C₁₋₆ alkyl group, a C₂₋₆ alkenyl group or a C₂₋₆ alkynyl group, and are unsubstituted or substituted by one or more substituents selected from halogen atoms and sulfonic acid groups. Preferably the C₁₋₆ alkyl group, C₂₋₆ alkenyl group and C₂-6 alkynyl group are unsubstituted.

Preferably in the first aspect, R_{8A} and R_{8B}, together with the nitrogen atom to which they are attached, form a 5- to 6-membered heteroaryl group or 5- to 6-membered heterocyclyl group.

More preferably in the first aspect, R_{8A} and R_{8B}, together with the nitrogen atom to which they are attached, form an imidazolyl group, a pyrazolyl group, a morpholinyl group, a piperidinyl group or a piperazinyl group.

Most preferably in the first aspect, R_{8A} and R_{8B}, together with the nitrogen atom to which they are attached, form an imidazolyl group (i.e., of a group of the formula

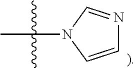 [SMILES: *n1ccnc1]

Preferably in the second aspect, R_{8A} and R_{8B} independently represent a C₁₋₄ alkyl group or a C₂₋₄ alkenyl group. More preferably in the second aspect, R_{8A} and R_{8B} independently represent a C₁₋₄ alkyl group. Most preferably in the second aspect, R₅ and R_{8B} independently represent a methyl group or an ethyl group.

In the present invention, therefore, preferably R_{8A} and R_{8B} either: (a) together with the nitrogen atom to which they are attached, form a 5- to 6-membered heteroaryl group or 5- to 6-membered heterocyclyl group; or (b) independently represent a C₁₋₄ alkyl group or a C₂₋₄ alkenyl group. More preferably R_{8A} and R_{8B} either: (a) together with the nitrogen atom to which they are attached, form an imidazolyl group, a pyrazolyl group, a morpholinyl group, a piperidinyl group or a piperazinyl group; or (b) independently represent a C₁₋₄ alkyl group. Most preferably, R_{8A} and R_{8B} either: (a) together with the nitrogen atom to which they are attached, form an imidazolyl group; or (b) independently represent a methyl group or an ethyl group—and optimally R_{8A} and R_{8B}, together with the nitrogen atom to which they are attached, form an imidazolyl group.

Exemplary cyclosporine analogues of the disclosure include those of formula (Ia) in which R₁ represents hydrogen, R₂ represents

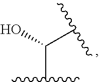

R₃ represents ethyl, R₄ represents methyl, R₅ represents —CH₂CH(CH₃)₂, R₆ represents ethylene (i.e. —(CH₂)₂—), R₇ represents hydrogen, R_{C} represents —C(O)O—, and R_{8A} and R_{8B}, together with the nitrogen atom to which they are attached, form an imidazolyl group. Further exemplary cyclosporine analogues of the disclosure are those where R₆ is —(CH₂)₃— or —(CH₂)₄—, particularly —(CH₂)₄—, rather than —(CH₂)₃—. In each of these exemplary cyclosporine analogues of the disclosure, preferred Ring A groups include those in which the C₆₋₁₀ arylene group or 5- to 10-membered heteroarylene group is a naphthylene group, a 9- to 10-membered heteroarylene group or a phenylene group. Furthermore, in these exemplary cyclosporine analogues of the disclosure, it is particularly preferred that, in ring A, said C₆₋₁₀ arylene group or 5- to 10-membered heteroarylene group is a naphthylene group. Still further in these exemplary cyclosporine analogues of the disclosure, preferred substituents of ring A, when ring A is substituted, are selected from C₁₋₄ alkyl, C₁₋₄ haloalkyl and C₁₋₄ alkoxy groups, most preferably C₁₋₄ alkyl groups. Preferably, Ring A is unsubstituted or substituted by one or two substituents, such as an unsubstituted naphthylene group (e.g. of formula (IIIa). Hence, one particular, exemplary compound is “BG147”, the formula of which is indicated in the Examples section. Another particular, exemplary compound is “JW3-158”, the formula of which is indicated in the Examples section. Still further exemplary compounds, the formulae of which are again indicated in the Examples section, are “BG150”, “BG181”, “BG185”, “BG186”, “BG190” and “BG149”. Still further exemplary compounds, the formulae of which are again indicated in the Examples section, are “VP72”, “VP73”, “VP74”, “VP50”, “VP53” and “VP76”.
Compounds in which —R_{C}—R₆—N(R_{8A})(R_{8B}) is defined according to (Y):

The disclosure also embraces compounds in which —R_{C}—R₆—N(R_{8A})(R_{8B}) is defined according to (Y), which means where —R_{C}—R₆—N(R_{8A})(R_{8B}) together form a group of formula (VI)

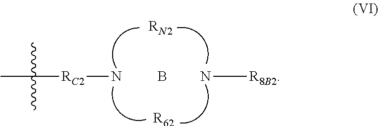

In formula (VI), R_{C2} represents —C(O)—, —S(O)₂—, —N(R_{N})—C(O)—, —N(R_{N})—C(S)—, —C(CF₃)— or —OC(O)—, and preferably represents —C(O)—.

In formula (VI), Ring B is a 5-10 membered heterocyclylene ring containing both the nitrogen atom bound to R_{C2} and the nitrogen atom bound to R_{8B2}, and in which R_{N2} and R₆₂ are each alkylene groups. Preferably Ring B is a 5-7 membered heterocyclylene ring containing both the nitrogen atom bound to R_{C2} and the nitrogen atom bound to R_{8B2}, and in which R_{N2} and R₆₂ are each alkylene groups. Particularly preferably Ring B is a 6 membered heterocyclylene ring containing both the nitrogen atom bound to R_{C2} and the nitrogen atom bound to R_{8B2}, and in which R_{N2} and R₆₂ are each alkylene groups. Most preferably Ring B is a membered heterocyclylene ring containing both the nitrogen atom bound to R_{C2} and the nitrogen atom bound to R_{8B2}, and in which R_{N2} and R₆₂ are each C₂ alkylene groups (i.e., Ring B is a 1,4-piperazinylene group).

In Formula (VI), R_{8B2} is a C₁₋₆ alkyl group, a C₂₋₆ alkenyl group or a C₂₋₆ alkynyl group, and is unsubstituted or substituted by one or more substituents selected from halogen atoms, sulfonic acid groups, and hydroxy groups. Preferably R_{8B2} is a C₁₋₄ alkyl group or a C₂₋₄ alkenyl group (and which is unsubstituted or substituted). More preferably R_{8B2} is a C₁₋₄ alkyl group which is unsubstituted or substituted. Most preferably, R_{8B2} is a methyl group or an ethyl group which is unsubstituted or substituted. Preferably R_{8B2} is unsubstituted or substituted with one or more substituents selected from halogen atoms and hydroxy groups. Most preferably R_{8B2} is unsubstituted or substituted with a hydroxy group. A particularly preferred R_{8B2} is hydroxyethyl (—CH₂CH₂OH).

One particular, exemplary such compound is “VP51”, the formula of which is indicated in the Examples section.

### Further Disclosure Relating to the Cyclosporine Analogues

As disclosed herein, in the present cyclosporine analogues the moiety —R_{C}—R₆—N(R_{8A})(R_{8B}) is defined according to either (X) or (Y). Preferably, however, the moiety —R_{C}—R₆—N(R_{8A})(R_{8B}) is defined according to (X). Furthermore, in this preferred aspect, the moiety —R_{C}—R₆—N(R_{8A})(R_{8B}) is preferably defined according to (X) and R_{8A} and R_{8B} either:
(a) together with the nitrogen atom to which they are attached, form a 5- to 10-membered heteroaryl group or 5- to 10-membered heterocyclyl group, wherein said heteroaryl group and said heterocyclyl group are either unsubstituted or substituted by one or more substituents selected from halogen atoms and C₁₋₆ alkyl, C₁₋₆ haloalkyl, C₁₋₆ alkoxy, —(C₁₋₆ alkyl)ₙC(O)O(C₁₋₆ alkyl) (where n=0 or 1), —(C₁₋₆ alkyl)ₙOC(O)(C₁₋₆ alkyl) (where n=0 or 1), C₁₋₆ alkylthiol, —N(R_{N})₂ (wherein each R_{N} independently represents a hydrogen atom or a C₁₋₆ alkyl group), —CN, —S(O)₂NH₂, nitro and sulfonic acid groups; or
(b) independently represent a C₁₋₆ alkyl group, a C₂₋₆ alkenyl group or a C₂₋₆ alkynyl group, and are unsubstituted or substituted by one or more substituents selected from halogen atoms and sulfonic acid groups.

Cyclosporine analogues according to the invention typically have low, minimal, or eliminated ability to bind to, inhibit and/or degrade CypA. For instance, the cyclosporine analogues according to the invention preferably have lower ability to bind to, inhibit and/or degrade CypA than CsA. In a preferred embodiment, a cyclosporine analogue of the invention has a lower binding affinity than cyclosporine A (CsA) to cyclophilin A (CypA), preferably less than a half of the binding affinity of CsA to CypA, more preferably less than a fifth of the binding affinity of CsA to CypA and most preferably less than a tenth of the binding affinity of CsA to CypA (e.g., when determined by a standard binding assay such as by surface plasmon resonance, SPR). Methods of determining relative ability to bind to, inhibit and/or degrade a protein (e.g. CypA) of one compound (e.g. a cyclosporine analogue according to the invention) relative to another (e.g., CsA) are well-known in the art and could routinely be utilised by those skilled in the art. One non-limiting method for determining ability to bind to CypA (e.g. as Kd (Kinetic), Kd (Affinity), Max Response, Koff, and/or Kon) is set out in Example 3.

Cyclosporine analogues according to the invention are typically IFITM3 inhibitors. For instance, in a preferred embodiment a cyclosporine analogue according to the invention may be a more potent IFITM3 inhibitor than CsA. Determination of IFITM3 inhibition, including with reference to CsA as a comparator, can be done using any method known in the art for measuring the ability of a compound to inibit a protein (e.g., in vitro). One suitable, non-limiting method is set in Example 2.

### Synthesis

Compounds of the invention may be prepared by standard methods known in the art. Representative examples of synthesis of analogues of cyclosporine, and which can be readily adapted to provide compounds of the present invention, are provided in WO 2021/229237 (including, but not limited to, the working examples of this publication); the content of WO 2021/229237 is herein incorporated by reference in its entirety. Further exemplary examples of synthesis of compounds is provided in the working examples of the present application.

Particularly useful intermediate compounds, and which therefore forms further aspects of the present invention, are the following
(a) the compound “VP75” whose synthesis is described in Example 8 and which has the following structure:

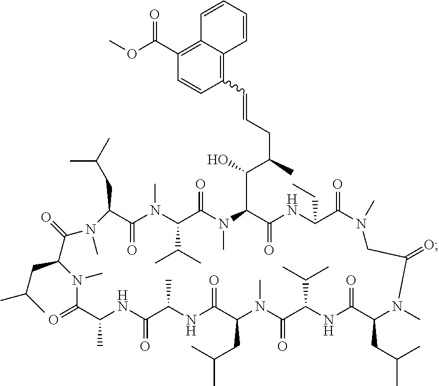 [SMILES: CC[C@@H]1NC(=O)[C@H]([C@H](O)[C@H](C)CC=Cc2ccc(C(=O)OC)c3ccccc23)N(C)C(=O)[C@H](C(C)C)N(C)C(=O)[C@H](CC(C)C)N(C)C(=O)[C@H](CC(C)C)N(C)C(=O)[C@@H](C)NC(=O)[C@H](C)NC(=O)[C@H](CC(C)C)N(C)C(=O)[C@H](C(C)C)NC(=O)[C@H](CC(C)C)N(C)C(=O)CN(C)C1=O]

and
(b) the compound “VP_130” whose synthesis is described in Example 10 and which has the following structure:

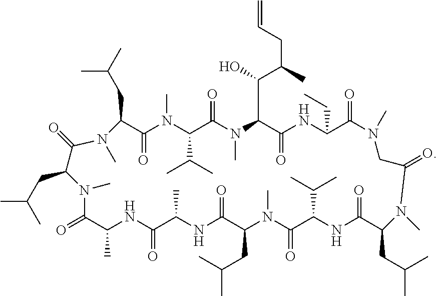 [SMILES: C=CC[C@@H](C)[C@@H](O)[C@H]1C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

These compounds are, in particular, especially useful for preparation of compounds of the invention in which:
R₁ represents hydrogen;

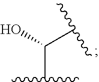

R₂ represents
R₃ represents ethyl;
R₄ represents methyl;
R₅ represents —CH₂CH(CH₃)₂; and
R₇ represents hydrogen.

The compound VP75 is specifically particularly useful for making compounds further characterised in that:
ring A represents

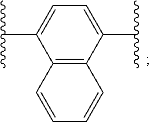 [SMILES: *c1ccc(*)c2ccccc12]

and
R_{C} represents —C(O)O— or —C(O)N—. The compound VP_130 can similarly be used for making such compounds, as well as other compounds of the present disclosure. Non-limiting, exemplary instances of synthesis, and subsequent reactions, of these compounds are provided in Examples 8 and 10.

### Gene Therapy Applications

The compounds of the present invention can be used in gene therapy, such as for increasing the efficiency of transduction of mammalian cells, human cells, human haematopoietic stem cells (HSC) and/or progenitor cells, induced human haematopoietic stem and/or progenitor cells, and/or cells differentiated from the human haematopoietic stem and/or progenitor cells or induced human haematopoietic stem and/or progenitor cells such as cells selected from one or more of a common myeloid progenitor, a megakaryocyte, an erythroblast, a mast cell, a myeloblast, a basophil, a neutrophil, an eosinophil, a monocyte, a common lymphoid progenitor, a natural killer cell, a T cell such as an α/β T cell, a γδ T cell or a regulatory T cell, a B cell and a plasma cell, by a gene therapy vector. The present invention embraces such uses of the compound, associated pharmaceutical compositions, and methods of treatment. Advantageously, it has been found that the compounds of the invention are easy-to-synthesise, highly potent IFITM3 inhibitors that can be utilised to enhance HIV-vector infection, reduce vector dose required and overcome patient variability. As further discussed elsewhere herein, the compounds may also have reduced binding to CypA compared with CsA, and therefore overcome a limitation associated with the previously described use of CsA, and other related cyclosporine analogues, in similar applications.

### Cells

A stem cell is able to differentiate into many cell types. A cell that is able to differentiate into all cell types is known as totipotent. In mammals, only the zygote and early embryonic cells are totipotent. Stem cells are found in most, if not all, multicellular organisms. They are characterised by the ability to renew themselves through mitotic cell division and differentiate into a diverse range of specialised cell types. The two broad types of mammalian stem cells are embryonic stem cells that are isolated from the inner cell mass of blastocysts, and adult stem cells that are found in adult tissues. In a developing embryo, stem cells can differentiate into all of the specialised embryonic tissues. In adult organisms, stem cells and progenitor cells act as a repair system for the body, replenishing specialised cells, but also maintaining the normal turnover of regenerative organs, such as blood, skin or intestinal tissues.

Haematopoietic stem cells (HSCs) are multipotent stem cells that may be found, for example, in peripheral blood, bone marrow and umbilical cord blood. HSCs are capable of self-renewal and differentiation into any blood cell lineage. They are capable of recolonising the entire immune system, and the erythroid and myeloid lineages in all the haematopoietic tissues (such as bone marrow, spleen and thymus). They provide for life-long production of all lineages of haematopoietic cells.

Haematopoietic progenitor cells have the capacity to differentiate into a specific type of cell. In contrast to stem cells however, they are already more specific: they are pushed to differentiate into their “target” cell. A difference between stem cells and progenitor cells is that stem cells can replicate indefinitely, whereas progenitor cells can only divide a limited number of times. Haematopoietic progenitor cells can be rigorously distinguished from HSCs only by functional in vivo assay (i.e. transplantation and demonstration of whether they can give rise to all blood lineages over prolonged time periods).

Preferably, the haematopoietic stem and progenitor cells of the invention comprise the CD34 cell surface marker (denoted as CD34+).

A population of haematopoietic stem and/or progenitor cells may be obtained from a tissue sample. For example, a population of haematopoietic stem and/or progenitor cells may be obtained from peripheral blood (e.g. adult and foetal peripheral blood), umbilical cord blood, bone marrow, liver or spleen. Preferably, these cells are obtained from peripheral blood or bone marrow. They may be obtained after mobilisation of the cells in vivo by means of growth factor treatment.

Mobilisation may be carried out using, for example, G-CSF, plerixaphor or combinations thereof. Other agents, such as NSAIDs and dipeptidyl peptidase inhibitors, may also be useful as mobilising agents.

With the availability of the stem cell growth factors GM-CSF and G-CSF, most haematopoietic stem cell transplantation procedures are now performed using stem cells collected from the peripheral blood, rather than from the bone marrow. Collecting peripheral blood stem cells provides a bigger graft, does not require that the donor be subjected to general anaesthesia to collect the graft, results in a shorter time to engraftment and may provide for a lower long-term relapse rate. Bone marrow may be collected by standard aspiration methods (either steady-state or after mobilisation), or by using next-generation harvesting tools (e.g. Marrow Miner). In addition, haematopoietic stem and/or progenitor cells may also be derived from induced pluripotent stem cells, and such cells are referred to here as induced haematopoietic stem and/or progenitor cells. Induced pluripotent stem cells, such as induced human pluripotent stem cells, may be generated by directly reprogramming a differentiated cell such as a fibroblast (see, for instance, Cell. 126 (4), 663-676, 2006; Cell, 131 (5), 861-872, 2007; Science, 318 (5858), 1917-1920, 2007; and Nat Biotechnol., 26 (1), 101-106, 2008). Induced pluripotent stem cell may be further differentiated to provide induced haematopoietic stem and/or progenitor cell. The induced haematopoietic stem and/or progenitor cell may be further differentiated to other cells of the haematopoietic lineage.

HSCs are typically of low forward scatter and side scatter profile by flow cytometric procedures. Some are metabolically quiescent, as demonstrated by Rhodamine labelling which allows determination of mitochondrial activity. HSCs may comprise certain cell surface markers such as CD34, CD45, CD133, CD90 and CD49f. They may also be defined as cells lacking the expression of the CD38 and CD45RA cell surface markers. However, expression of some of these markers is dependent upon the developmental stage and tissue-specific context of the HSC. Some HSCs called “side population cells” exclude the Hoechst 33342 dye as detected by flow cytometry. Thus, HSCs have descriptive characteristics that allow for their identification and isolation.

CD38 is the most established and useful single negative marker for human HSCs. Human HSCs may also be negative for lineage markers such as CD2, CD3, CD14, CD16, CD19, CD20, CD24, CD36, CD56, CD66b, CD271 and CD45RA. However, these markers may need to be used in combination for HSC enrichment. By “negative marker” it is to be understood that human HSCs lack the expression of these markers.

CD34 and CD133 are the most useful positive markers for HSCs. Some HSCs are also positive for lineage markers such as CD90, CD49f and CD93. However, these markers may need to be used in combination for HSC enrichment. By “positive marker” it is to be understood that human HSCs express these markers.

A differentiated cell is a cell which has become more specialised in comparison to a stem cell or progenitor cell. Differentiation occurs during the development of a multicellular organism as the organism changes from a single zygote to a complex system of tissues and cell types. Differentiation is also a common process in adults: adult stem cells divide and create fully-differentiated daughter cells during tissue repair and normal cell turnover. Differentiation dramatically changes a cell's size, shape, membrane potential, metabolic activity and responsiveness to signals. These changes are largely due to highly-controlled modifications in gene expression. In other words, a differentiated cell is a cell which has specific structures and performs certain functions due to a developmental process which involves the activation and deactivation of specific genes.

Here, a differentiated cell includes differentiated cells of the haematopoietic lineage such as monocytes, macrophages, neutrophils, basophils, eosinophils, erythrocytes, megakaryocytes/platelets, dendritic cells, T-cells, B-cells and NK-cells. For example, differentiated cells of the haematopoietic lineage can be distinguished from stem cells and progenitor cells by detection of cell surface molecules which are not expressed or are expressed to a lesser degree on undifferentiated cells. Examples of suitable human lineage markers include CD33, CD13, CD14, CD15 (myeloid), CD19, CD20, CD22, CD79a (B), CD36, CD71, CD235a (erythroid), CD2, CD3, CD4, CD8 (T) and CD56 (NK).

By “isolated population” of cells it is to be understood that the population of cells has been previously removed from the body. An isolated population of cells may be cultured and manipulated ex vivo or in vitro using standard techniques known in the art. An isolated population of cells may later be reintroduced into a subject. Said subject may be the same subject from which the cells were originally isolated or a different subject. Methods and uses carried out on isolated populations of cells are ex vivo or in vitro methods and uses.

A population of cells may be purified selectively for cells that exhibit a specific phenotype or characteristic, and from other cells which do not exhibit that phenotype or characteristic, or exhibit it to a lesser degree. For example, a population of cells that expresses a specific marker (such as CD34) may be purified from a starting population of cells. Alternatively, or in addition, a population of cells that does not express another marker (such as CD38) may be purified.

By “enriching” a population of cells for a certain type of cells it is to be understood that the concentration of that type of cells is increased within the population. The concentration of other types of cells may be concomitantly reduced.

Purification or enrichment may result in the population of cells being substantially pure of other types of cell. Purifying or enriching for a population of cells expressing a specific marker (e.g. CD34 or CD38) may be achieved by using an agent that binds to that marker, preferably substantially specifically to that marker.

An agent that binds to a cellular marker may be an antibody, for example an anti-CD34 or anti-CD38 antibody. The term “antibody” refers to complete antibodies or antibody fragments capable of binding to a selected target, and including Fv, ScFv, F(ab′) and F(ab′)2, monoclonal and polyclonal antibodies, engineered antibodies including chimeric, CDR-grafted and humanised antibodies, and artificially selected antibodies produced using phage display or alternative techniques. In addition, alternatives to classical antibodies may also be used in the invention, for example “avibodies”, “avimers”, “anticalins”, “nanobodies” and “DARPins”.

The agents that bind to specific markers may be labelled so as to be identifiable using any of a number of techniques known in the art. The agent may be inherently labelled, or may be modified by conjugating a label thereto. By “conjugating” it is to be understood that the agent and label are operably linked. This means that the agent and label are linked together in a manner which enables both to carry out their function (e.g. binding to a marker, allowing fluorescent identification, or allowing separation when placed in a magnetic field) substantially unhindered. Suitable methods of conjugation are well known in the art and would be readily identifiable by the skilled person.

A label may allow, for example, the labelled agent and any cell to which it is bound to be purified from its environment (e.g. the agent may be labelled with a magnetic bead, or an affinity tag, such as avidin), detected or both. Detectable markers suitable for use as a label include fluorophores (e.g. green, cherry, cyan and orange fluorescent proteins) and peptide tags (e.g. His tags, Myc tags, FLAG tags and HA tags).

A number of techniques for separating a population of cells expressing a specific marker are known in the art. These include magnetic bead-based separation technologies (e.g. closed-circuit magnetic bead-based separation), flow cytometry, fluorescence-activated cell sorting (FACS), affinity tag purification (e.g. using affinity columns or beads, such biotin columns to separate avidin-labelled agents) and microscopy-based techniques. It may also be possible to perform the separation using a combination of different techniques, such as a magnetic bead-based separation step followed by sorting of the resulting population of cells for one or more additional (positive or negative) markers by flow cytometry. Clinical grade separation may be performed, for example, using the CliniMACS® system (Miltenyi). This is an example of a closed-circuit magnetic bead-based separation technology.

It is also envisaged that dye exclusion properties (e.g. side population or rhodamine labelling) or enzymatic activity (e.g. ALDH activity) may be used to enrich for HSCs.

The cells of the present invention may be formulated for administration to subjects with a pharmaceutically acceptable carrier, diluent or excipient. Suitable carriers and diluents include isotonic saline solutions, for example phosphate-buffered saline, and potentially contain human serum albumin. B Handling of the cell therapy product is preferably performed in compliance with FACT-JACIE International Standards for cellular therapy.

### Vectors

A vector is a tool that allows or facilitates the transfer of an entity from one environment to another. Vectors that are used in the present invention to transduce mammalian cells, human cells, haematopoietic stem and/or progenitor cells, induced haematopoietic stem and/or progenitor cells, and/or cells differentiated from the haematopoietic stem and/or progenitor cells or induced haematopoietic stem and/or progenitor cells such as cells selected from one or more of a common myeloid progenitor, a megakaryocyte, an erythroblast, a mast cell, a myeloblast, a basophil, a neutrophil, an eosinophil, a monocyte, a common lymphoid progenitor, a natural killer cell, a T cell such as an α/β T cell, a γδ T cell or a regulatory T cell, a B cell and a plasma cell, may be viral vectors. The viral vectors may be derived from HIV-1, HIV-2, SIV, FIV, BIV, EIAV, CAEV or visna lentivirus. These viruses are all lentiviruses. By “vector derived from” a certain type of virus, it is to be understood that the vector comprises at least one component part derivable from that type of virus.

A retroviral vector may be derived from or may be derivable from any suitable retrovirus. A large number of different retroviruses have been identified. Examples include murine leukaemia virus (MLV), human T-cell leukaemia virus (HTLV), mouse mammary tumour virus (MMTV), Rous sarcoma virus (RSV), Fujinami sarcoma virus (FuSV), Moloney murine leukaemia virus (Mo-MLV), FBR murine osteosarcoma virus (FBR MSV), Moloney murine sarcoma virus (Mo-MSV), Abelson murine leukaemia virus (A-MLV), avian myelocytomatosis virus-29 (MC29) and avian erythroblastosis virus (AEV). A detailed list of retroviruses may be found in Coffin, J. M. et al. (1997) Retroviruses, Cold Spring Harbour Laboratory Press, 758-63.

Retroviruses may be broadly divided into two categories, “simple” and “complex”. Retroviruses may be even further divided into seven groups. Five of these groups represent retroviruses with oncogenic potential. The remaining two groups are the lentiviruses and the spumaviruses. A review of these retroviruses is presented in Coffin, J. M. et al. (1997) Retroviruses, Cold Spring Harbour Laboratory Press, 758-63.

The basic structure of retrovirus and lentivirus genomes share many common features such as a 5′ LTR and a 3′ LTR. Between or within these are located a packaging signal to enable the genome to be packaged, a primer binding site, integration sites to enable integration into a host cell genome, and gag, pol and env genes encoding the packaging components—these are polypeptides required for the assembly of viral particles.

Lentiviruses have additional features, such as rev and RRE sequences in HIV, which enable the efficient export of RNA transcripts of the integrated provirus from the nucleus to the cytoplasm of an infected target cell.

In the provirus, these genes are flanked at both ends by regions called long terminal repeats (LTRs). The LTRs are responsible for proviral integration and transcription. LTRs also serve as enhancer-promoter sequences and can control the expression of the viral genes.

The LTRs themselves are identical sequences that can be divided into three elements: U3, R and U5. U3 is derived from the sequence unique to the 3′ end of the RNA. R is derived from a sequence repeated at both ends of the RNA. U5 is derived from the sequence unique to the 5′ end of the RNA. The sizes of the three elements can vary considerably among different retroviruses.

In a defective retroviral vector genome gag, pol and env may be absent or not functional.

In a typical retroviral vector, at least part of one or more protein coding regions essential for replication may be removed from the virus. This makes the viral vector replication-defective. Portions of the viral genome may also be replaced by a library encoding candidate modulating moieties operably linked to a regulatory control region and a reporter moiety in the vector genome in order to generate a vector comprising candidate modulating moieties which is capable of transducing a target host cell and/or integrating its genome into a host genome.

Lentivirus vectors are part of the larger group of retroviral vectors. A detailed list of lentiviruses may be found in Coffin, J. M. et al. (1997) Retroviruses, Cold Spring Harbour Laboratory Press, 758-63. In brief, lentiviruses can be divided into primate and non-primate groups. Examples of primate lentiviruses include but are not limited to human immunodeficiency virus (HIV), the causative agent of human acquired immunodeficiency syndrome (AIDS); and simian immunodeficiency virus (SIV). Examples of non-primate lentiviruses include the prototype “slow virus” visna/maedi virus (VMV), as well as the related caprine arthritis-encephalitis virus (CAEV), equine infectious anaemia virus (EIAV), and the more recently described feline immunodeficiency virus (FIV) and bovine immunodeficiency virus (BIV).

The lentivirus family differs from retroviruses in that lentiviruses have the capability to infect both dividing and non-dividing cells (Lewis, P et al. (1992) EMBO J. 11:3053-8; Lewis, P. F. et al. (1994) J. Virol. 68:510-6). In contrast, other retroviruses, such as MLV, are unable to infect non-dividing or slowly dividing cells such as those that make up, for example, muscle, brain, lung and liver tissue.

A lentiviral vector, as used herein, is a vector which comprises at least one component part derivable from a lentivirus. Preferably, that component part is involved in the biological mechanisms by which the vector infects cells, expresses genes or is replicated.

The lentiviral vector may be a “primate” vector. The lentiviral vector may be a “non-primate” vector (i.e. derived from a virus which does not primarily infect primates, especially humans). Examples of non-primate lentiviruses may be any member of the family of lentiviridae which does not naturally infect a primate.

As examples of lentivirus-based vectors, HIV-1- and HIV-2-based vectors are described below.

The HIV-1 vector contains cis-acting elements that are also found in simple retroviruses. It has been shown that sequences that extend into the gag open reading frame are important for packaging of HIV-1. Therefore, HIV-1 vectors often contain the relevant portion of gag in which the translational initiation codon has been mutated. In addition, most HIV-1 vectors also contain a portion of the env gene that includes the RRE. Rev binds to RRE, which permits the transport of full-length or singly spliced mRNAs from the nucleus to the cytoplasm. In the absence of Rev and/or RRE, full-length HIV-1 RNAs accumulate in the nucleus. Alternatively, a constitutive transport element from certain simple retroviruses such as Mason-Pfizer monkey virus can be used to relieve the requirement for Rev and RRE. Efficient transcription from the HIV-1 LTR promoter requires the viral protein Tat.

Most HIV-2-based vectors are structurally very similar to HIV-1 vectors. Similar to HIV-1-based vectors, HIV-2 vectors also require RRE for efficient transport of the full-length or singly spliced viral RNAs.

In one system, the vector and helper constructs are from two different viruses, and the reduced nucleotide homology may decrease the probability of recombination. In addition to vectors based on the primate lentiviruses, vectors based on FIV have also been developed as an alternative to vectors derived from the pathogenic HIV-1 genome. The structures of these vectors are also similar to the HIV-1 based vectors.

Preferably the viral vector used in the present invention has a minimal viral genome. By “minimal viral genome” it is to be understood that the viral vector has been manipulated so as to remove the non-essential elements and to retain the essential elements in order to provide the required functionality to infect, transduce and deliver a nucleotide sequence of interest to a target host cell. Further details of this strategy can be found in WO 1998/017815.

Preferably the plasmid vector used to produce the viral genome within a host cell/packaging cell will have sufficient lentiviral genetic information to allow packaging of an RNA genome, in the presence of packaging components, into a viral particle which is capable of infecting a target cell, but is incapable of independent replication to produce infectious viral particles within the final target cell. Preferably the vector lacks a functional gag-pol and/or env gene and/or other genes essential for replication.

However, the plasmid vector used to produce the viral genome within a host cell/packaging cell will also include transcriptional regulatory control sequences operably linked to the lentiviral genome to direct transcription of the genome in a host cell/packaging cell. These regulatory sequences may be the natural sequences associated with the transcribed viral sequence (i.e. the 5′ U3 region), or they may be a heterologous promoter, such as another viral promoter (e.g. the CMV promoter).

The vectors may be self-inactivating (SIN) vectors in which the viral enhancer and promoter sequences have been deleted. SIN vectors can be generated and transduce non-dividing cells in vivo with an efficacy similar to that of wild-type vectors. The transcriptional inactivation of the long terminal repeat (LTR) in the SIN provirus should prevent mobilisation by replication-competent virus. This should also enable the regulated expression of genes from internal promoters by eliminating any cis-acting effects of the LTR.

The vectors may be integration-defective. Integration defective lentiviral vectors (IDLVs) can be produced, for example, either by packaging the vector with catalytically inactive integrase (such as an HIV integrase bearing the D64V mutation in the catalytic site; Naldini, L. et al. (1996) Science 272:263-7; Naldini, L. et al. (1996) Proc. Natl. Acad. Sci. USA 93:11382-8; Leavitt, A. D. et al. (1996) J. Virol. 70:721-8) or by modifying or deleting essential at sequences from the vector LTR (Nightingale, S. J. et al. (2006) Mol. Ther. 13:1121-32), or by a combination of the above.

HIV-derived vectors for use in the present invention are not particularly limited in terms of HIV strain. Numerous examples of sequences of HIV strains may be found at the HIV Sequence Database (http://www.hiv.lanl.gov/content index). For example, a HIV-1-derived vector may be derived from any of the HIV-1 strains NL4-3, IIIB LAI or HXB2_LAI (X4-tropic), or BAL (R₅-tropic), or a chimaera thereof. A HIV-2-derived vector may be derived, for example, from the HIV-2 strain ROD.

As discussed elsewhere herein, cyclosporine analogues according to the invention have low, minimal, or eliminated ability to bind to, inhibit and/or degrade CypA. Many viral vectors utilised in gene therapy applications recruit CypA to assist with efficient infection (e.g., the efficient infection of HSCs) because it shields the capsid from restriction by another antiviral protein called TRIM5. Inhibition of CypA recruitment by the cyclosporine analogues consequently risks reducing the efficiency of transduction to suboptimal levels.

In one embodiment, a viral vector that is not sensitive, or has limited sensitivity, to CypA is utilised (e.g. a viral vector that does not bind to CypA). Non-limiting examples of such vectors include HIV capsid mutants that are insensitive to CypA and resistant to TRIM5 restriction such as A92E and G94D (see, for instance, Ylinen et al., Journal of Virology 83 (4), 2009, p. 2044-2047).

However, in a more common embodiment of the invention, a viral vector that is sensitive to CypA is utilised (e.g., a viral vector that does bind to CypA). For instance, the viral vector may be a vector that is not selected from HIV capsid mutants that are insensitive to CypA and resistant to TRIM5 restriction, such as A92E and G94D.

### Transduction of Cells

In one aspect, the present invention provides the use of a cyclosporine analogue according to the invention for increasing the efficiency of transduction of an isolated population of mammalian cells, human cells, human haematopoietic stem and/or progenitor cells, induced human haematopoietic stem and/or progenitor cells, and/or cells differentiated from the human haematopoietic stem and/or progenitor cells or induced human haematopoietic stem and/or progenitor cells such as cells selected from one or more of a common myeloid progenitor, a megakaryocyte, an erythroblast, a mast cell, a myeloblast, a basophil, a neutrophil, an eosinophil, a monocyte, a common lymphoid progenitor, a natural killer cell, a T cell such as an α/β T cell, a γδ T cell or a regulatory T cell, a B cell and a plasma cell, by a vector derived from HIV-1, HIV-2, SIV, FIV, BIV, EIAV, CAEV or visna lentivirus.

Increasing the efficiency of transduction refers to an increase in the transduction of mammalian cells, human cells, haematopoietic stem and/or progenitor cells, induced haematopoietic stem and/or progenitor cells, and/or cells differentiated from the haematopoietic stem and/or progenitor cells or induced haematopoietic stem and/or progenitor cells such as cells selected from one or more of a common myeloid progenitor, a megakaryocyte, an erythroblast, a mast cell, a myeloblast, a basophil, a neutrophil, an eosinophil, a monocyte, a common lymphoid progenitor, a natural killer cell, a T cell such as an α/β T cell, a γδ T cell or a regulatory T cell, a B cell and a plasma cell, in the presence of an agent (e.g. a cyclosporine analogue according to the invention), in comparison to the transduction achieved in the absence of the agent but under otherwise substantially identical conditions. An increased efficiency of transduction may therefore allow the multiplicity of infection (MOI) and/or the transduction time required to achieve effective transduction to be reduced.

In one embodiment, the percentage of mammalian cells, human cells, haematopoietic stem and/or progenitor cells, induced haematopoietic stem and/or progenitor cells, and/or cells differentiated from the haematopoietic stem and/or progenitor cells or induced haematopoietic stem and/or progenitor cells such as cells selected from one or more of a common myeloid progenitor, a megakaryocyte, an erythroblast, a mast cell, a myeloblast, a basophil, a neutrophil, an eosinophil, a monocyte, a common lymphoid progenitor, a natural killer cell, a T cell such as an α/β T cell, a γδ T cell or a regulatory T cell, a B cell and a plasma cell transduced by the vector is increased. In another embodiment, the vector copy number per cell is increased. Preferably both are achieved at the same time.

Methods for determining the percentage of cells transduced by a vector are known in the art. Suitable methods include flow cytometry, fluorescence-activated cell sorting (FACS) and fluorescence microscopy. The technique employed is preferably one which is amenable to automation and/or high throughput screening.

For example, a population of cells may be transduced with a vector which harbours a reporter gene. The vector may be constructed such that the reporter gene is expressed when the vector transduces a cell. Suitable reporter genes include genes encoding fluorescent proteins, for example green, yellow, cherry, cyan or orange fluorescent proteins. Once the population of cells has been transduced by the vector, both the number of cells expressing and not-expressing the reporter gene may be quantified using a suitable technique, such as FACS. The percentage of cells transduced by the vector may then be calculated.

Alternatively, quantitative PCR (qPCR) may be used to determine the percentage of cells transduced by a vector that does not harbour a reporter gene. For example, single colonies of CD34+ cells may be picked from a semi-solid culture and qPCR may be performed on each colony separately to determine the percentage of vector-positive colonies among those analysed.

Methods for determining vector copy number are also known in the art. The technique employed is preferably one which is amenable to automation and/or high throughput screening. Suitable techniques include quantitative PCR (qPCR) and Southern blot-based approaches.

The concentration at which a cyclosporine analogue according to the invention can be applied to a population of mammalian cells, human cells, haematopoietic stem and/or progenitor cells, induced haematopoietic stem and/or progenitor cells, and/or cells differentiated from the haematopoietic stem and/or progenitor cells or induced haematopoietic stem and/or progenitor cells such as cells selected from one or more of a common myeloid progenitor, a megakaryocyte, an erythroblast, a mast cell, a myeloblast, a basophil, a neutrophil, an eosinophil, a monocyte, a common lymphoid progenitor, a natural killer cell, a T cell such as an α/β T cell, a γδ T cell or a regulatory T cell, a B cell and a plasma cell, may be adjusted for different vector systems to optimise transduction efficiency. Methods for determining transduction efficiency have been described above.

A cyclosporine analogue according to the invention may be toxic to mammalian cells, human cells, haematopoietic stem and/or progenitor cells, induced haematopoietic stem and/or progenitor cells, and/or cells differentiated from the haematopoietic stem and/or progenitor cells or induced haematopoietic stem and/or progenitor cells such as cells selected from one or more of a common myeloid progenitor, a megakaryocyte, an erythroblast, a mast cell, a myeloblast, a basophil, a neutrophil, an eosinophil, a monocyte, a common lymphoid progenitor, a natural killer cell, a T cell such as an α/β T cell, a γδ T cell or a regulatory T cell, a B cell and a plasma cell, if it is applied at too high a concentration. The toxicity the cyclosporine analogue according to the invention on mammalian cells, human cells, haematopoietic stem and/or progenitor cells, induced haematopoietic stem and/or progenitor cells, and/or cells differentiated from the haematopoietic stem and/or progenitor cells or induced haematopoietic stem and/or progenitor cells such as cells selected from one or more of a common myeloid progenitor, a megakaryocyte, an erythroblast, a mast cell, a myeloblast, a basophil, a neutrophil, an eosinophil, a monocyte, a common lymphoid progenitor, a natural killer cell, a T cell such as an α/β T cell, a γδ T cell or a regulatory T cell, a B cell and a plasma cell, may be determined by quantifying the number of viable cells remaining after exposure to the cyclosporine analogue for a certain time. Methods for quantifying the number of viable cells are known in the art. A skilled person may therefore select a suitable concentration of a cyclosporine analogue according to the invention to maximise increase in transduction efficiency while minimising the effect of toxicity using the approaches described herein.

For example, the concentration of the cyclosporine analogue applied to the population of mammalian cells, human cells, haematopoietic stem and/or progenitor cells, induced haematopoietic stem and/or progenitor cells, and/or cells differentiated from the haematopoietic stem and/or progenitor cells or induced haematopoietic stem and/or progenitor cells such as cells selected from one or more of a common myeloid progenitor, a megakaryocyte, an erythroblast, a mast cell, a myeloblast, a basophil, a neutrophil, an eosinophil, a monocyte, a common lymphoid progenitor, a natural killer cell, a T cell such as an α/β T cell, a γδ T cell or a regulatory T cell, a B cell and a plasma cell, may be about 0.1-50 μM, about 1-50 μM, about 5-50 μM, about 10-50 μM, about 5-40 μM, about 10-40 μM, about 10-25 μM, or about 10-15 μM.

The present invention encompasses the use of a cyclosporine analogue according to the invention. The cyclosporine analogue of the present invention may be those which increase the efficiency of transduction of an isolated population of mammalian cells, human cells, haematopoietic stem and/or progenitor cells, induced haematopoietic stem and/or progenitor cells, and/or cells differentiated from the haematopoietic stem and/or progenitor cells or induced haematopoietic stem and/or progenitor cells such as cells selected from one or more of a common myeloid progenitor, a megakaryocyte, an erythroblast, a mast cell, a myeloblast, a basophil, a neutrophil, an eosinophil, a monocyte, a common lymphoid progenitor, a natural killer cell, a T cell such as an α/β T cell, a γδ T cell or a regulatory T cell, a B cell and a plasma cell, by a vector derived from HIV-1, HIV-2, SIV, FIV, BIV, EIAV, CAEV or visna lentivirus.

Cyclosporine analogues according to the invention are preferably of low toxicity for mammals, in particular human, and preferably are of low toxicity for haematopoietic stem and/or progenitor cells, induced haematopoietic stem and/or progenitor cells, and/or cells differentiated from the haematopoietic stem and/or progenitor cells or induced haematopoietic stem and/or progenitor cells such as cells selected from one or more of a common myeloid progenitor, a megakaryocyte, an erythroblast, a mast cell, a myeloblast, a basophil, a neutrophil, an eosinophil, a monocyte, a common lymphoid progenitor, a natural killer cell, a T cell such as an α/β T cell, a γδ T cell or a regulatory T cell, a B cell and a plasma cell.

### Therapeutic Applications: Gene Therapy and Transplantation

The vector used in the present invention preferably comprises a nucleotide of interest (NOI). Preferably the NOI gives rise to a therapeutic effect and therefore has utility in gene therapy. Suitable NOIs include, but are not limited to sequences encoding enzymes, cytokines, chemokines, hormones, antibodies, anti-oxidant molecules, engineered immunoglobulin-like molecules, single chain antibodies, fusion proteins, immune co-stimulatory molecules, immunomodulatory molecules, anti-sense RNA, microRNA, shRNA, siRNA, bozymes, miRNA target sequences, a transdomain negative mutant of a target protein, toxins, conditional toxins, antigens, tumour suppressor proteins, growth factors, transcription factors, membrane proteins, surface receptors, anti-cancer molecules, vasoactive proteins and peptides, anti-viral proteins and hbozymes, and derivatives thereof (such as derivatives with an associated reporter group). The NOIs may also encode pro-drug activating enzymes. An example of a NOI is the beta-globin chain which may be used for gene therapy of thalassemia/sickle cell disease.

NOIs may also include those useful for the treatment of other diseases requiring nonurgent/elective gene correction in the myeloid lineage such as: chronic granulomatous disease (CGD, e.g. the gp91 phox transgene), leukocyte adhesion defects, other phagocyte disorders in patients without ongoing severe infections and inherited bone marrow failure syndromes (e.g. Fanconi anaemia), as well as primary immunodeficiencies (SCIDs). NOIs may also include those useful in the treatment of lysosomal storage disorders and immunodeficiencies.

The present invention also provides a population of mammalian cells, human cells, haematopoietic stem and/or progenitor cells, induced haematopoietic stem and/or progenitor cells, and/or cells differentiated from the haematopoietic stem and/or progenitor cells or induced haematopoietic stem and/or progenitor cells such as cells selected from one or more of a common myeloid progenitor, a megakaryocyte, an erythroblast, a mast cell, a myeloblast, a basophil, a neutrophil, an eosinophil, a monocyte, a common lymphoid progenitor, a natural killer cell, a T cell such as an α/β T cell, a γδ T cell or a regulatory T cell, a B cell and a plasma cell, prepared according to a method of the invention for use in therapy, for example for use in gene therapy. The use may be as part of a mammalian cell, human cell, haematopoietic stem and/or progenitor cell, induced haematopoietic stem and/or progenitor cells, and/or common myeloid progenitor, megakaryocyte, erythroblast, mast cell, myeloblast, basophil, neutrophil, eosinophil, monocyte, common lymphoid progenitor, natural killer cell, T cell such as α/β T cell, γδ T cell or regulatory T cell, B cell and plasma cell, transplantation procedure.

Haematopoietic stem cell transplantation (HSCT) is the transplantation of blood stem cells that may be derived from the bone marrow (in this case known as bone marrow transplantation) or blood. Stem cell transplantation is a medical procedure in the fields of haematology and oncology, most often performed for people with diseases of the blood or bone marrow, or certain types of cancer. Many recipients of HSCTs are multiple myeloma or leukaemia patients who would not benefit from prolonged treatment with, or are already resistant to, chemotherapy. Candidates for HSCTs include paediatric cases where the patient has an inborn defect such as severe combined immunodeficiency or congenital neutropenia with defective stem cells, and also children or adults with aplastic anaemia who have lost their stem cells after birth. Other conditions treated with stem cell transplants include sickle-cell disease, myelodysplastic syndrome, neuroblastoma, lymphoma, Ewing's Sarcoma, Desmoplastic small round cell tumour and Hodgkin's disease. More recently non-myeloablative, or so-called “mini transplant”, procedures have been developed that require smaller doses of preparative chemotherapy and radiation. This has allowed HSCT to be conducted in the elderly and other patients who would otherwise be considered too weak to withstand a conventional treatment regimen.

In one embodiment, a population of mammalian cells, human cells, haematopoietic stem and/or progenitor cells, induced haematopoietic stem and/or progenitor cells, and/or cells differentiated from the haematopoietic stem and/or progenitor cells or induced haematopoietic stem and/or progenitor cells such as cells selected from one or more of a common myeloid progenitor, a megakaryocyte, an erythroblast, a mast cell, a myeloblast, a basophil, a neutrophil, an eosinophil, a monocyte, a common lymphoid progenitor, a natural killer cell, a T cell such as an α/β T cell, a γδ T cell or a regulatory T cell, a B cell and a plasma cell, prepared according to a method of the invention is administered as part of an autologous stem cell transplant procedure.

In another embodiment, a population of mammalian cells, human cells, haematopoietic stem and/or progenitor cells, induced haematopoietic stem and/or progenitor cells, and/or cells differentiated from the haematopoietic stem and/or progenitor cells or induced haematopoietic stem and/or progenitor cells such as cells selected from one or more of a common myeloid progenitor, a megakaryocyte, an erythroblast, a mast cell, a myeloblast, a basophil, a neutrophil, an eosinophil, a monocyte, a common lymphoid progenitor, a natural killer cell, a T cell such as an α/β T cell, a γδ T cell or a regulatory T cell, a B cell and a plasma cell, prepared according to a method of the invention is administered as part of an allogeneic stem cell transplant procedure.

By “autologous stem cell transplant procedure” it is to be understood that the starting population of cells (which are then transduced according to a method of the invention) is obtained from the same subject as that to which the transduced cell population is administered. Autologous transplant procedures are advantageous as they avoid problems associated with immunological incompatibility and are available to subjects irrespective of the availability of a genetically matched donor.

By “allogeneic stem cell transplant procedure” it is to be understood that the starting population of cells (which are then transduced according to a method of the invention) is obtained from a different subject as that to which the transduced cell population is administered. Preferably, the donor will be genetically matched to the subject to which the cells are administered to minimise the risk of immunological incompatibility.

Suitable doses of transduced cell populations are such as to be therapeutically and/or prophylactically effective. The dose to be administered may depend on the subject and condition to be treated, and may be readily determined by a skilled person.

The products, methods and uses of the present invention may be useful in the treatment of the disorders listed in WO 1998/005635. For ease of reference, part of that list is now provided: cancer, inflammation or inflammatory disease, dermatological disorders, fever, cardiovascular effects, haemorrhage, coagulation and acute phase response, cachexia, anorexia, acute infection, HIV infection, shock states, graft-versus-host reactions, autoimmune disease, reperfusion injury, meningitis, migraine and aspirin-dependent anti-thrombosis; tumour growth, invasion and spread, angiogenesis, metastases, malignant, ascites and malignant pleural effusion; cerebral ischaemia, ischaemic heart disease, osteoarthritis, rheumatoid arthritis, osteoporosis, asthma, multiple sclerosis, neurodegeneration, Alzheimer's disease, atherosclerosis, stroke, vasculitis, Crohn's disease and ulcerative colitis; periodontitis, gingivitis; psoriasis, atopic dermatitis, chronic ulcers, epidermolysis bullosa; corneal ulceration, retinopathy and surgical wound healing; rhinitis, allergic conjunctivitis, eczema, anaphylaxis; restenosis, congestive heart failure, endometriosis, atherosclerosis or endosclerosis.

In addition, or in the alternative, the products, methods and uses of the present invention may be useful in the treatment of the disorders listed in WO 1998/007859. For ease of reference, part of that list is now provided: cytokine and cell proliferation/differentiation activity; immunosuppressant or immunostimulant activity (e.g. for treating immune deficiency, including infection with human immune deficiency virus; regulation of lymphocyte growth; treating cancer and many autoimmune diseases, and to prevent transplant rejection or induce tumour immunity); regulation of haematopoiesis, e.g. treatment of myeloid or lymphoid diseases; promoting growth of bone, cartilage, tendon, ligament and nerve tissue, e.g. for healing wounds, treatment of burns, ulcers and periodontal disease and neurodegeneration; inhibition or activation of follicle-stimulating hormone (modulation of fertility); chemotactic/chemokinetic activity (e.g. for mobilising specific cell types to sites of injury or infection); haemostatic and thrombolytic activity (e.g. for treating haemophilia and stroke); anti-inflammatory activity (for treating e.g. septic shock or Crohn's disease); as antimicrobials; modulators of e.g. metabolism or behaviour; as analgesics; treating specific deficiency disorders; in treatment of e.g. psoriasis, in human or veterinary medicine.

In addition, or in the alternative, the products, methods and uses of the present invention may be useful in the treatment of the disorders listed in WO 1998/009985. For ease of reference, part of that list is now provided: macrophage inhibitory and/or T cell inhibitory activity and thus, anti-inflammatory activity; anti-immune activity, i.e. inhibitory effects against a cellular and/or humoral immune response, including a response not associated with inflammation; inhibit the ability of macrophages and T cells to adhere to extracellular matrix components and fibronectin, as well as up-regulated fas receptor expression in T cells; inhibit unwanted immune reaction and inflammation including arthritis, including rheumatoid arthritis, inflammation associated with hypersensitivity, allergic reactions, asthma, systemic lupus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynaecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of stokes, post-polio syndrome, immune and inflammatory components of psychiatric disorders, myelitis, encephalitis, subacute sclerosing panencephalitis, encephalomyelitis, acute neuropathy, subacute neuropathy, chronic neuropathy, Guillaim-Barre syndrome, Sydenham chora, myasthenia gravis, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery, bone marrow transplantation or other transplantation complications and/or side effects, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue.

In addition, or in the alternative, the products, methods and uses of the present invention may be useful in the treatment of B-thalassemia, chronic granulomatous disease, metachromatic leukodystrophy, mucopolysaccharidoses disorders and other lysosomal storage disorders.

Gene therapy may occur through the sustained or transient release of product encoded by the NOI, for example an encoded product set out above. For example, haematopoietic progenitor cells generally provide short term engraftment. Accordingly, gene therapy by administering transduced haematopoietic progenitor cells may provide a non-permanent effect in the subject. For example, the effect may be limited to 1-6 months following administration of the transduced haematopoietic progenitor cells. An advantage of this approach would be better safety and tolerability, due to the self-limited nature of the therapeutic intervention. Such haematopoietic progenitor cell gene therapy may be suited to treatment of acquired disorders, for example cancer, where time-limited expression of a (potentially toxic) anti-cancer nucleotide of interest may be sufficient to eradicate the disease. In contrast, HSCs may be more likely to provide long term engraftment, and therefore may be better suited to providing a longer term effect in the subject or an effect sustained throughout the lifetime of the subject. For example, the effect may be limited to 3 months to 30 years following administration of the transduced HSCs. A longer term or sustained effect may be suited to treatment of inherited genetic disorders, for example SCID, where long term expression of a nucleotide of interest may be desirable.

Following gene therapy, the encoded product may be released systemically in the subject, for example into the circulation. Systemic release may result in a 1.1, 1.2, 1.5, 2, 5, 10, 25, 50, 100, 250, 500 or 1000 fold increase in encoded product activity relative to the activity before gene therapy. Assays for measuring encoded product activity would be apparent to the skilled person.

Alternatively, following gene therapy, the encoded product may be released in a targeted fashion such that it is targeted to a specific group of tissue and/or organ. For example, the encoded product may be targeted to the central nervous system (CNS), heart, face, mouth, eye, bone, liver, spleen and/or lung. Targeted release may result in a 1.1, 1.2, 1.5, 2, 5, 10, 25, 50, 100, 250, 500 or 1000 fold increase in encoded product activity in the targeted tissue and/or organ relative to the activity in the same tissue and/or organ before gene therapy. In addition, or in the alternative, targeted release may result in a 1.1, 1.2, 1.5, 2, 5, 10, 25, 50, 100, 250, 500 or 1000 fold increase in encoded product activity in the targeted tissue and/or organ after gene therapy, relative to the activity in non-targeted tissues and/or organs. Assays for measuring encoded product activity would be apparent to the skilled person.

Thus, the invention provides means whereby pathological phenotypes associated with the indications provided above can be corrected, treated, arrested, palliated and/or prevented. Correction can refer to both partial, total correction and hyper-correction. Correction may be achieved after about 10 days, 20 days, 30 days, 40 days, 50 days, 60 days, 70 days, 80 days, 90 days, 100 days, 125 days, 150 days, 175 days, 200 days, 250 days, 300 days, 1 year, 1.5 years, 2 years, 2.5 year, 3 years, 4 year or 5 years. The effect of correcting, treating, arresting, palliating and/or preventing a phenotype may be transient. Alternatively, the effect of correcting, treating, arresting, palliating and/or preventing a phenotype may be long term, or sustained.

The treatment of mammals, particularly humans, is preferred. However, both human and veterinary treatments may be within the scope of the present invention.

### Kit

In another aspect, the present invention provides a kit comprising the cyclosporine analogue and/or cell populations of the invention. The cyclosporine analogue and/or cell populations of the invention may be provided in suitable containers. The kit may also include instructions for use.

Treatment of Viral Infections and Other Pathological Conditions Associated with IFITM3 Expression

Cyclosporine A, CsA, has previously been disclosed as having antiviral activity, including against coronaviruses (see, e.g.: de Wilde et al., J Gen Virol. 2011; 92 (Pt 11): 2542-2548; Carbajo-Lozoya et al., Virus Res. 2014 184:44-53; Nasiri et al., J Dermatolog Treat. 2020:1-6; de Wilde et al., Virus Res. 2017 15; 228:7-13). More recently, it has been proposed that IFITM proteins may actively promote SARS-Cov-2 infections, for instance by hijacking these usually anti-viral proteins for efficient viral infection. This raises the prospect of treating such viral infections by inhibiting IFITM, thereby disrupting this hijacking mechanism and decreasing the efficiency of viral infection.

The compounds of the invention are hence useful in the treatment or prevention of a viral infection in a patient. Typically, the patient is a mammal, such as a human or a cat, preferably a human.

Typically, the viral infection is human immunodeficiency virus-1 (HIV-1), influenza virus, human cytomegalovirus (hCMV), hepatitis C virus (HCV), dengue virus, a vaccinia virus (such as Small Pox), feline immunodeficiency virus (FIV) or a corona virus (such as COVID-19 or SARs). Preferably, the viral infection is COVID-19, human immunodeficiency virus-1 (HIV-1), influenza virus, human cytomegalovirus (hCMV) or hepatitis C virus (HCV), more preferably COVID-19 or human immunodeficiency virus-1 (HIV-1) and more preferably still COVID-19.

COVID-19 (i.e., coronavirus disease 2019 is the disease caused by severe acute respiratory syndrome coronavirus 2 (SARS-COV-2). Many variants of COVID-19 have been identified and reached significant levels of prevalence in the global population, including the Alpha, Delta and Omicron variants. As shown in the examples section of this disclosure, the compounds of the invention may be especially useful in the treatment of certain variants of COVID-19 (for instance, the Delta and/or Omicron variant, especially the Omicron variant), which, without being limited by theory, may have evolved to make use of IFITM proteins for their efficient survival and/or replication in infected hosts.

The compounds of the invention may be administered to humans in various manners such as oral, rectal, vaginal, parenteral, intramuscular, intraperitoneal, intraarterial, intrathecal, intrabronchial, subcutaneous, intradermal, intravenous, nasal, buccal or sublingual routes of administration. The particular mode of administration and dosage regimen will be selected by the attending physician, taking into account a number of factors including the age, weight and condition of the patient.

The compound is typically administered as a pharmaceutical composition, which generally comprises a derivative of the invention and a pharmaceutically acceptable excipient, diluent or carrier. Thus, pharmaceutical compositions that contain the compounds of the invention will normally be formulated with an appropriate pharmaceutically acceptable excipient, carrier or diluent depending upon the particular mode of administration being used. For instance, parenteral formulations are usually injectable fluids that use pharmaceutically and physiologically acceptable fluids such as physiological saline, balanced salt solutions, or the like as a vehicle. Oral formulations, on the other hand, may be solids, e.g. tablets or capsules, or liquid solutions or suspensions.

Compositions may be formulated in unit dosage form, i.e., in the form of discrete portions containing a unit dose, or a multiple or sub-unit of a unit dose.

The amount of the compound of the invention that is given to a patient will depend upon on the activity of the particular compound in question. Further factors include the condition being treated, the nature of the patient under treatment and the severity of the condition under treatment. The timing of administration of the compound should be determined by medical personnel. As a skilled physician will appreciate, and as with any drug, the compound may be toxic at very high doses. For example, the compound may be administered at a dose of from 0.01 to 30 mg/kg body weight, such as from 0.1 to 10 mg/kg, more preferably from 0.1 to 5 mg/kg body weight.

The compounds of the invention may be given alone or in combination with one or more additional anti-viral agents, preferably one or more agents useful for treating human immunodeficiency virus-1 (HIV-1), influenza virus, human cytomegalovirus (hCMV), hepatitis C virus (HCV), dengue virus, vaccinia virus, feline immunodeficiency virus (FIV) or corona virus.

One such anti-viral agent is Remdesivir.

Anti-viral agents useful for treating HIV-1 include non-nucleoside reverse transcriptase inhibitors (NNRTIs), nucleoside analogue reverse transcriptase inhibitor (NRTIs) and nucleotide analog reverse-transcriptase inhibitors (NtRTIs). Preferred NRTIs include Zidovudine, Didanosine, Zalcitabine, Stavudine, Lamivudine, Abacavir, Emtricitabine, Entecavir and Apricitabine, Preferred NNRTIs include Efavirenz, Nevirapine, Delavirdine, Etravirine and Rilpivirine. Preferred NtRTIs include Tenofovir and Adefovir.

Anti-viral agents useful for treating influenza virus include (a) neuraminidase inhibitors such as oseltamivir and zanamivir, and (b) M2 protein inhibitors such as amantadine and rimantadine.

Anti-viral agents useful for treating human cytomegalovirus (hCMV) include human cytomegalovirus antibodies and antiviral agents such as Ganciclovir, Valganciclovir, Foscarnet and cidofovir.

Anti-viral agents useful for treating hepatitis C virus (HCV) include pegylated interferon alpha and ribavirin.

Anti-viral agents useful for treating vaccinia virus include cidofovir.

Anti-viral agents useful for treating feline immunodeficiency virus (FIV) include Lymphocyte T-Cell Immunomodulator.

Anti-viral agents useful for treating coronaviruses, including COVID-19, include dexamethasone, remdesivir, Paxlovid, sotrovimab, bebtelovimab, REGEN-COV, bamlanivimab/etesevimab, and molnupiravir.

The active ingredients are typically administered as a combined preparation.

Accordingly, the present invention also provides a combination comprising a compound of the invention and one or more said additional anti-viral agents. The combination is typically for use in the treatment or prevention of said viral infection in a patient.

The invention further provides a compound of the invention for use in the treatment or prevention of a viral infection in a patient, by co-administration with one or more said additional anti-viral agents. Co-administration can be simultaneous, concurrent, separate or sequential.

The invention further provides one or more additional said anti-viral agents, for use in the treatment or prevention of a said viral infection in a patient, by co-administration with a compound of the invention. Co-administration can be simultaneous, concurrent, separate or sequential.

The present invention further provides a product comprising a compound of the invention and one or more said additional anti-viral agents, as a combined preparation for simultaneous, concurrent, separate or sequential use in the treatment or prevention of a said viral infection in a patient.

The compounds of the invention, being IFITM3 inhibitors, also find utility more generally in the treatment of pathological conditions associated with IFITM3 expression. The term “associated with IFITM3 expression” can be used interchangeably herein with “susceptible to treatment by inhibition of IFITM3”. Non-limiting examples of such pathological conditions include viral infections, such as those discussed elsewhere herein, and Alzheimer's disease. However, it being known that IFITM3 has roles in regulating innate immune signaling, the compounds of the invention can be useful as medicaments in any clinical situation where manipulating IFITM3 favorably impacts on immunity.

### EXAMPLES

### Example 1: Representative Synthesis of Certain Exemplary Compounds

### 2-(1H-imidazol-1-yl)ethyl-4-((4R,5R,E)-5-((2S,5S,11S,14S,17S,20S,23R,26S,29S,32S)-5-ethyl-11,17,26,29-tetraisobutyl-14,32-diisopropyl-1,7,10,16,20,23,25,28,31-nonamethyl-3,6,9,12,15,18,21,24,27,30,33-undecaoxo-1,4,7,10,13,16,19,22,25,28,31-undecaazacyclotritriacontan-2-yl)-5-hydroxy-4-methylpent-1-en-1-yl)benzoate (JW3-158)

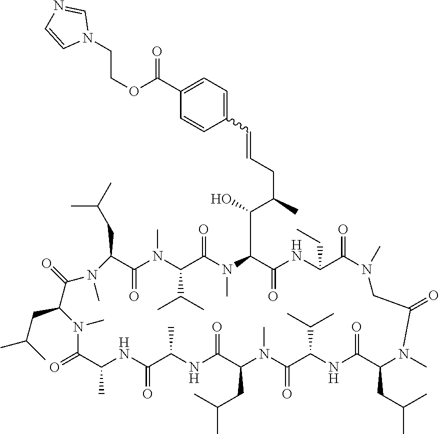 [SMILES: CC[C@@H]1NC(=O)[C@H]([C@H](O)[C@H](C)CC=Cc2ccc(C(=O)OCCn3ccnc3)cc2)N(C)C(=O)[C@H](C(C)C)N(C)C(=O)[C@H](CC(C)C)N(C)C(=O)[C@H](CC(C)C)N(C)C(=O)[C@@H](C)NC(=O)[C@H](C)NC(=O)[C@H](CC(C)C)N(C)C(=O)[C@H](C(C)C)NC(=O)[C@H](CC(C)C)N(C)C(=O)CN(C)C1=O]

To a solution of Cyclosporin A (500 mg, 0.42 mmol) in DCM (5 mL) was added 4-vinylbenzoic acid (185 mg, 1.25 mmol) and Hoveyda-Grubbs 2^{nd} generation catalyst (17 mol %). The reaction mixture was stirred in a MW reactor (90° C., 40 min) and then allowed to cool. The solvent was removed under reduce pressure and the row material was redissolved in MeOH, passed through a Stratospheres PL Thiol MP SPE cartridge (polymer Lab, Varian Inc) to remove the catalyst and concentrated under vacuum. The crude product (32 mg, 0.024 mmol) was dissolved in DCM (1 mL) and 1-(2-hydroxyethyl) imidazole (4 mg, 0.036 mmol), EDC (12 mg, 0.063 mmol) and 4-pyrrolidinopyridine (0.5 mg, 0.003 mmol) were added. After stirring for 15 h at r.t., the solvent was removed under vacuum and the crude was purified by reverse phase C18 (10→100% MeCN 0.1% FA in H₂O 0.1% FA) to give the final product (13 mg, 0.009 mmol, 38%) as a white solid.

HRMS (m/z): found 1402.91; calc. for C₇₃H₁₁₉N₁₃O₁₄ [MH]⁺: 1402.83.

### 2-(1H-imidazol-1-yl)-ethyl-4-((4R,5R,E)-5-((2S,5S,11S,14S,17S,20S,23R,26S,29S,32S)-5-ethyl-11,17,26,29-tetraisobutyl-14,32-diisopropyl-1,7,10,16,20,23,25,28,31-nonamethyl-3,6,9,12,15,18,21,24,27,30,33-undecaoxo-1,4,7,10,13,16,19,22,25,28,31-undecaazacyclotritriacontan-2-yl)-5-hydroxy-4-methylpent-1-en-1-yl)-1-naphthoate (BG147)

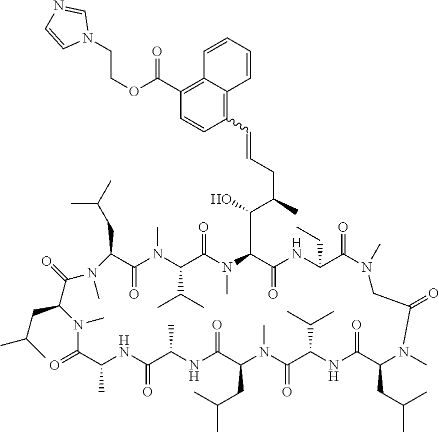 [SMILES: CC[C@@H]1NC(=O)[C@H]([C@H](O)[C@H](C)CC=Cc2ccc(C(=O)OCCn3ccnc3)c3ccccc23)N(C)C(=O)[C@H](C(C)C)N(C)C(=O)[C@H](CC(C)C)N(C)C(=O)[C@H](CC(C)C)N(C)C(=O)[C@@H](C)NC(=O)[C@H](C)NC(=O)[C@H](CC(C)C)N(C)C(=O)[C@H](C(C)C)NC(=O)[C@H](CC(C)C)N(C)C(=O)CN(C)C1=O]

Methyl 4-bromo-1-naphthoate (530 mg, 1.99 mmol) was dissolved in a solution THF:Water=9:1 (28 mL) followed by potassium vinyl trifluoroborate (1.34 g, 9.99 mmol), caesium carbonate (1.95 g, 5.99 mmol) and the catalyst Pd(dppf)Cl₂·DCM (5 mol %). The reaction mixture was stirred at 70° C. for 15 h, warmed to r.t. and diluted with H₂O. The organic layer was separated and the aqueous phase extracted with DCM (×3). The combined organic layers were washed with Brine (×3), dried over Mg₂SO₄, filtered and concentrated under reduced pressure. The crude product was purified by chromatography column on silica gel (EtOAc 0%→100% in Cyclohexane) to obtain methyl 4-vinyl-1-naphthoate (376 mg, 1.78 mmol, 89%) as a yellow oil. Methyl 4-vinyl-1-naphthoate (11 mg, 0.053 mmol) was dissolved into anhydrous DCE followed by Cyclosporine A (32 mg, 0.026 mmol) and Hoveyda-Grubbs 2^{nd} generation catalyst (4 mol %). The reaction mixture was stirred in a MW reactor (70° C., 30 min) and then cooled to r.t. The solvent was removed under reduce pressure and the row material was redissolved in MeOH, passed through a Stratospheres PL Thiol MP SPE cartridge (polymer Lab, Varian Inc) to remove the catalyst and concentrated under vacuum to give the product as a white solid (36 mg). NaH (21 mg, 0.52 mmol) was added to a two-necked round bottomed flask, sealed, and flushed with N₂, to which anhydrous THF was then added (18 mL) with stirring. This mixture was cooled to 0° C. and 2-(1H-imidazol-1-yl) ethan-1-ol (118 mg, 1.06 mmol) was added. After stirring for 15 min, a solution of the previous crude compound in anhydrous THF was added and the mixture was stirred at r.t overnight. After this time, the reaction mixture was quenched with saturated ammonium chloride solution (1 mL) and concentrated under vacuum. The crude residue was then dissolved into DCM (20 mL), washed with H₂O (3×20 mL) and concentrated under vacuum. Purification by reverse phase C18 (0->100% MeCN 0.1% FA+15% MeOH in H₂O 0.1% FA) gave the final product BG147 (3 mg, 0.002 mmol, 7%, cis/trans mixture) as a colourless solid.

LCMS (m/z): found 1452.9; calc. for C₇₇H₁₂₁N₁₃O₁₄ 1452.9 [M+H⁺].

¹H-NMR (600 MHz, MeOD, 8 ppm, J Hz) δ 8.90 (d, Napthyl Aromatic, 1H), 6.43 (m, Alkenyl, 1H), 5.93 (d, Alkenyl, 1H), 3.19 (s, NMe, 3H), 3.13 (s, NMe, 3H), 3.05 (s, NMe, 3H), 3.01 (s, NMe, 3H), 2.97 (s, NMe, 3H), 2.90 (s, NMe, 4H), 2.82-2.78 (s, NMe, 4H). (Characteristic ¹H-NMR signals for BG147)

### ((4R,5R,E)-5-((2S,5S,11S,14S,17S,20S,23R,26S,29S,32S)-5-ethyl-11,17,26,29-tetraisobutyl-14,32-diisopropyl-1,7,10,16,20,23,25,28,31-nonamethyl-3,6,9,12,15,18,21,24,27,30,33-undecaoxo-1,4,7,10,13,16,19,22,25,28,31-undecaazacyclotritriacontan-2-yl)-5-hydroxy-4-methylpent-1-en-1-yl)trifluoroborate (BG158)

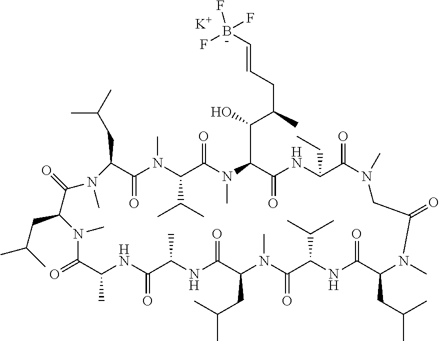

To a solution of Cyclosporin A (1 g, 0.83 mmol) in DCE (10 mL) was added vinylboronic acid pinacol ester (58 mg, 0.38 mmol) and Hoveyda-Grubbs 2nd generation catalyst (6 mol %). The reaction mixture was stirred in a MW reactor (70° C., 30 min) under N₂ atmosphere and then allowed to cool. The solvent was removed under reduce pressure and the row material was dissolved in MeOH, passed through a Stratospheres PL Thiol MP SPE cartridge (polymer Lab, Varian Inc) to remove the catalyst and concentrated under vacuum. The crude product (1.1 g, 0.83 mmol) was dissolved in MeOH (10 mL) and potassium hydrogen fluoride solution (4.5 M, 1 mL) was added. The reaction mixture was allowed to stir at r.t. overnight, concentrated under reduced pressure, dissolved in acetone (10 mL×3) and filtered. The filtrate was then precipitated into Et₂O (50 mL) to give an orange solid which was collected by filtration, washed with Et₂O (50 mL) and concentrated under reduced pressure to give the final product (50 mg, 0.04 mmol, 5%).

### 2-(1H-imidazol-1-yl)ethyl 4-((4R,5R)-5-((2S,5S,11S,14S,17S,20S,23R,26S,29S,32S)-5-ethyl-11,17,26,29-tetraisobutyl-14,32-diisopropyl-1,7,10,16,20,23,25,28,31-nonamethyl-3,6,9,12,15,18,21,24,27,30,33-undecaoxo-1,4,7,10,13,16,19,22,25,28,31-undecaazacyclotritriacontan-2-yl)-5-hydroxy-4-methylpent-1-en-1-yl)-3,5-dimethoxybenzoate (BG186)

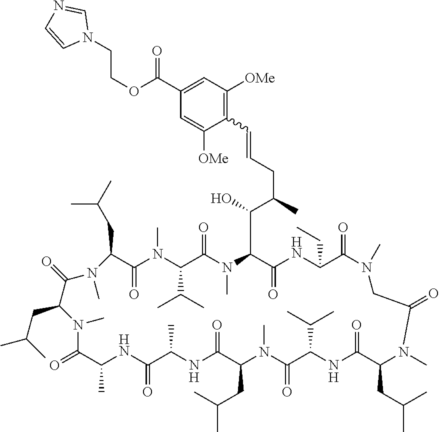 [SMILES: CC[C@@H]1NC(=O)[C@H]([C@H](O)[C@H](C)CC=Cc2c(OC)cc(C(=O)OCCn3ccnc3)cc2OC)N(C)C(=O)[C@H](C(C)C)N(C)C(=O)[C@H](CC(C)C)N(C)C(=O)[C@H](CC(C)C)N(C)C(=O)[C@@H](C)NC(=O)[C@H](C)NC(=O)[C@H](CC(C)C)N(C)C(=O)[C@H](C(C)C)NC(=O)[C@H](CC(C)C)N(C)C(=O)CN(C)C1=O]

BG158 (6 mg, 0.005 mmol) was dissolved in a solution THF:Water=9:1 (1.4 mL) followed by 2-(1H-imidazol-1-yl)ethyl 4-bromo-3,5-dimethoxybenzoate (3 g, 0.01 mmol), caesium carbonate (9 mg, 0.03 mmol) and the catalyst Pd(dppf)Cl₂·DCM (10 mol %). The reaction mixture was stirred at 90° C. for 30 min under N₂ atmosphere, warmed to r.t. and diluted with H₂O. The organic layer was separated and the aqueous phase extracted with DCM (×3). The combined organic layers were washed with Brine (×3), dried over Mg₂SO₄, filtered and concentrated under reduced pressure. The crude product was purified by reverse phase C18 (0->100% MeCN 0.1% FA+15% MeOH in H₂O 0.1% FA) obtaining the final product BG186 (0.2 mg, 3%).

### Example 2—JW3-158 is a More Potent Transduction Enhancer than Control Compounds CsA, CsH and JW115 in a Model System

HP-1 cells (human monocytic cell line) were treated with 10 ng/ml Type I interferon (IFNβ) overnight to induce IFITM3 expression. The following day the cells were infected with VSV-G pseudotyped HIV-1 vector encoding GFP (HIV-GFP) at a multiplicity of infection (MOI) of 0.2 IU/cell. Cells were infected in the presence of compound at 5 μM or equivalent volume of DMSO vehicle. 48 hours later infection levels were determined by counting GFP positive green cells by flow cytometry.

Infection of cells without IFN treatment is high and infection is lowered by IFN (baseline). Inhibitors rescue infection from IFN by inhibiting the anti-viral action of IFITM3. The higher the measured value (in fold rescue compared to baseline), the higher the rescue of infection, and the better the inhibition of IFITM3. Measured fold rescue for tested compound compared to inhibited baseline were 24.6 for CsH, 9.9 for CsA, 29.2 for JW3-158 and 14.2 for JW-115.

Note that this experiment shows that if cells are in an antiviral state due to IFN exposure, then the molecule improves infection. This is expected to even out differences in transduction efficiency between patients because variation is likely influenced by the level of antiviral state of the patient cells.

JW-115 was disclosed in WO 2021/229237 and has the structure:

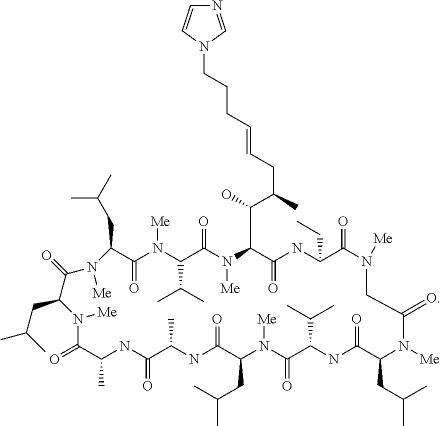

### Example 3—Representative Compounds have Reduced Binding to CypA Compared to CsA

### SPR Binding Experiments—Cyp Inhibitors

CypA binding was carried out using surface plasmon resonance as described (Warne J Biol Chem. 2016 Feb. 26; 291 (9): 4356-73). Cyclosporine A was used as the positive control.

### Protocol

Surface Plasmon Resonance (SPR) using Biacore at 25° C. was used to study the binding interactions between CypA and various inhibitors. The SPR binding experiments used a duel flow cell with a blank reference. The sensor chip surface was initially activated with a mixture of N-hydroxysuccinimide (NHS,) and N-(3-dimethyl-aminopropyl)-N′-ethyl-carbodiimide hydrochloride (EDC) for 420 s with a flow rate of 10 μl/min on blank flow cell 1 in addition to flow cell 2.

CypA protein was diluted to a concentration of 50 μg/mL by the addition of 10 mM sodium acetate pH 5 solution before immobilisation and covalent attachment to the carboxy-methylated dextran matrix of the Biacore CM5 sensor chip. CypA was run against the surface for 420 s and then any unreacted surface was quenched by 420 s of ethanolamine.

Immobilisations were carried out in the presence of JW47 (Warne J Biol Chem. 2016 Feb. 26; 291 (9): 4356-73). Cyp inhibitor compound stocks (10 mM concentration) in DMSO were diluted with HBS-EP+ buffer to give final DMSO concentrations of 2% (e.g. 5 μl into 245 μl of buffer).

### Kinetics/Affinity Experiments

Contact time of inhibitor vs surface was 120 s followed by a dissociation time of 600 s, with the latter being extended to 1200 s in the case of very strong binders such as CsA. No regeneration stage was required if the dissociation time was sufficient.

Solvent correction was carried out in each experiment using 8 different DMSO dilutions as reference: 2.8%, 2.6%, 2.4%, 2.2%, 2%, 1.8%, 1.6% & 1.5%.

A 50/50 mixture of buffer and DMSO is required to include a wash cycle in the experiment.

### EXAMPLE

50 μl of CypA (0.1 mg/ml)+40 μL of 10 mM sodium acetate pH5+10 μl of 600 μM of JW47 DMSO solution. This mixture was allowed to stand at room temperature for 10 mins prior to set-up in the injection rack.

Cyp inhibitor compound stocks (10 mM concentration) in DMSO were diluted with HBS-EP+ buffer to give final DMSO concentrations of 2% (e.g. 5 μl into 245 μl of buffer).

### Results

Results are summarized in the table below.

| Run | | Kd | Kd | Max | | |
|---|---|---|---|---|---|---|
| Order: | meta-R- | (Kinetics) | (Affinity) | Response | K_{off} | Kₒₙ |
| Sample | Group | uM | uM | (RU) | (s⁻¹) | (M⁻¹s⁻¹) |
| 1: CsA | — | — | — | ~40 | — | — |
| (Control) | | | | | | |
| 2: JW3-158 | H | 0.16 | 1.83 | 27.0 | 0.002168 | 13380 |
| 3: BG147 | Napthyl | 2.22 | 5.27 | 14.0 | 0.02164 | 9752 |
| 4: BG149 | Ethyl | 0.1 | 34.65 | 4.8 | 0.001098 | 11300 |
| 5: BG150 | Methyl | 3.84 | 1.31 | 18.7 | 0.04251 | 11070 |
| 6: BG181 | Methoxy | 35.9 | 2.66 | 9.8 | 0.2282 | 6356 |
| 7: BG185 | Isopropoxy | 259.5 | 2.92 | 14.5 | 0.5843 | 2252 |
| 8: BG186 | 3,5-Dimethoxy | 28.9 | 4.76 | 7.6 | 0.4221 | 14560 |
| 9: BG190 | CF₃ | 28.8 | 6.07 | 13.2 | 0.01382 | 4799 |

The structures of JW3-158, BG147 and BG186 are shown in Example 1. The structures of BG149, BG150, BG181, BG185 and BG190 are shown below.

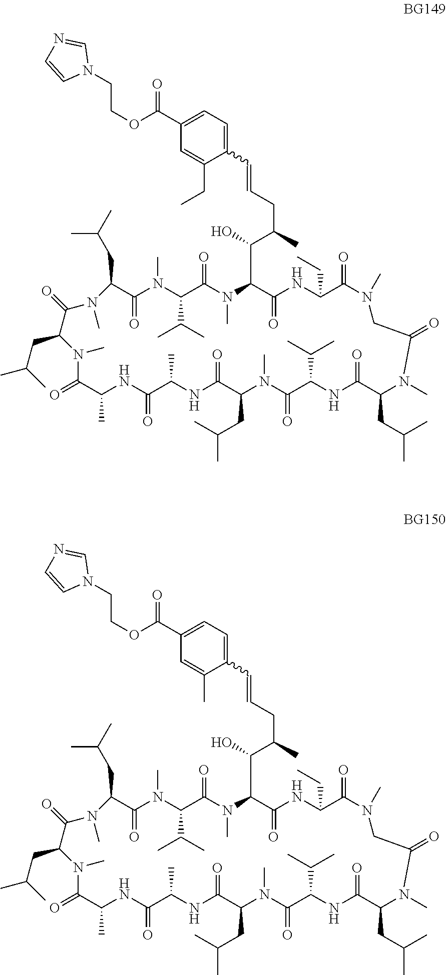 [SMILES: CCc1cc(C(=O)OCCn2ccnc2)ccc1C=CC[C@@H](C)[C@@H](O)[C@H]1C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C ; CC[C@@H]1NC(=O)[C@H]([C@H](O)[C@H](C)CC=Cc2ccc(C(=O)OCCn3ccnc3)cc2C)N(C)C(=O)[C@H](C(C)C)N(C)C(=O)[C@H](CC(C)C)N(C)C(=O)[C@H](CC(C)C)N(C)C(=O)[C@@H](C)NC(=O)[C@H](C)NC(=O)[C@H](CC(C)C)N(C)C(=O)[C@H](C(C)C)NC(=O)[C@H](CC(C)C)N(C)C(=O)CN(C)C1=O]

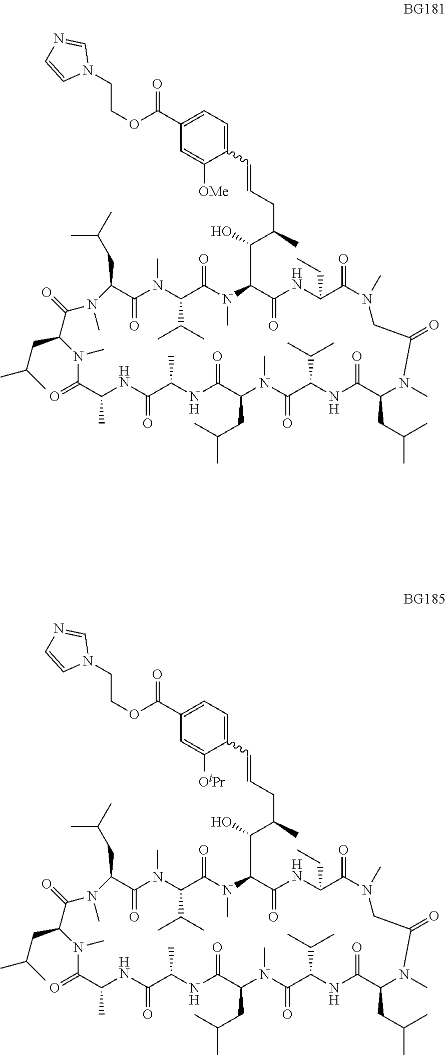 [SMILES: CC[C@@H]1NC(=O)[C@H]([C@H](O)[C@H](C)CC=Cc2ccc(C(=O)OCCn3ccnc3)cc2OC)N(C)C(=O)[C@H](C(C)C)N(C)C(=O)[C@H](CC(C)C)N(C)C(=O)[C@H](CC(C)C)N(C)C(=O)[C@@H](C)NC(=O)[C@H](C)NC(=O)[C@H](CC(C)C)N(C)C(=O)[C@H](C(C)C)NC(=O)[C@H](CC(C)C)N(C)C(=O)CN(C)C1=O ; CC[C@@H]1NC(=O)[C@H]([C@H](O)[C@H](C)CC=Cc2ccc(C(=O)OCCn3ccnc3)cc2OC(C)C)N(C)C(=O)[C@H](C(C)C)N(C)C(=O)[C@H](CC(C)C)N(C)C(=O)[C@H](CC(C)C)N(C)C(=O)[C@@H](C)NC(=O)[C@H](C)NC(=O)[C@H](CC(C)C)N(C)C(=O)[C@H](C(C)C)NC(=O)[C@H](CC(C)C)N(C)C(=O)CN(C)C1=O]

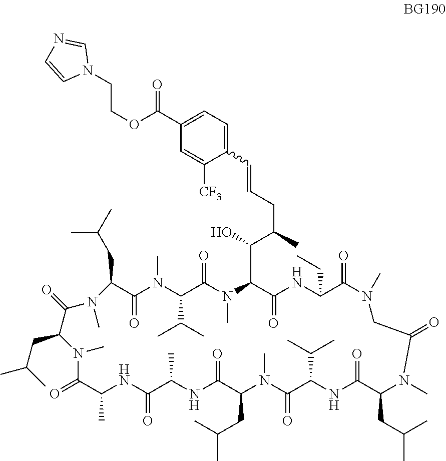 [SMILES: CC[C@@H]1NC(=O)[C@H]([C@H](O)[C@H](C)CC=Cc2ccc(C(=O)OCCn3ccnc3)cc2C(F)(F)F)N(C)C(=O)[C@H](C(C)C)N(C)C(=O)[C@H](CC(C)C)N(C)C(=O)[C@H](CC(C)C)N(C)C(=O)[C@@H](C)NC(=O)[C@H](C)NC(=O)[C@H](CC(C)C)N(C)C(=O)[C@H](C(C)C)NC(=O)[C@H](CC(C)C)N(C)C(=O)CN(C)C1=O]

### Example 4—Complementary Cell-Line Assays to Identify Selective IFITM3 Inhibitors

An experiment similar to that in Example 2 was performed in IFN treated THP-1 cells.

THP-1 cells (human monocytic cell line) were treated with 10 ng/ml type 1 interferon (IFNβ) overnight to induce IFITM3 expression. The following day the cells were infected with VSV-G pseudotyped HIV-1 vector encoding GFP (HIV-GFP) at a multiplicity of infection (MOI) of 0.35. Cells were infected in the presence of compound at 3 doses of 1.25, 2.5 and 5 μM or DMSO vehicle at 0.1% v/v. 48 hours later infection levels were determined by counting GFP positive green cells by flow cytometry.

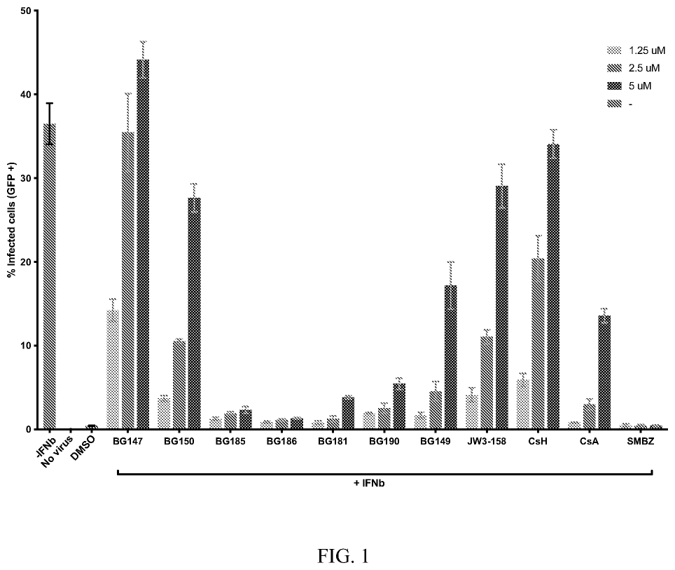

Results are shown in FIG. 1. The no virus bar leads to no infected (green) cells. Infection of cells without IFN treatment is high (dark grey bar) and infection is lowered by IFN (third bar). Inhibitors rescue infection from IFN by inhibiting the anti-viral action of IFITM3. The higher the bar, the higher the rescue of infection, the better the inhibition of IFITM3. BG147 completely rescued infection from IFN in this experiment and outperformed the other molecules. For each compound, three concentrations were tested (left to right bars being 1.25 μM, 2.5 UM and 5 μM, respectively).

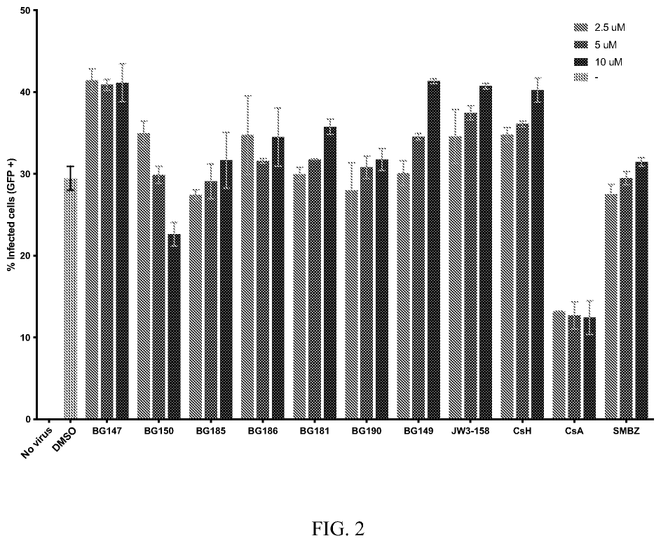

Next, an experiment was performed in U87 cells. These cells differ from THP-1 in that they express IFITM3 and TRIM5. Thus only inhibitors with reduced CypA inhibition will rescue infection. This functionally tests whether the inhibitors have lost CypA inhibition, but retain IFITM3 inhibition. Results are shown in FIG. 2.

U87 cells were infected at an MOI of 0.3 in the presence of DMSO (0.1% v/v) or inhibitors at 2.5, 5, or 10 μM (left bar to right bar for each indicated compound, respectively). Inhibition of IFITM3 raises infectivity. Unlike other inhibitors, BG147 gives maximal infection enhancement at the lowest concentration of 2.5 μM. Other molecules achieve maximal infection rescue at the highest concentration.

### Explanation for Differences in Drug Potency in Different Cell Lines

Cyclosporine A is a complex molecule that targets multiple pathways in cells. Specifically, CsA targets Cyclophilin A (an enzyme which is inhibited by CCsA) and IFITM3 (an antiviral membrane protein that is re-routed to degradation pathways in lysosomes on addition of CsA). Cyclophilin A (CypA) acts as a cofactor for HIV infection.

It acts to protect HIV from the anti-viral protein TRIM5. Therefore, inhibiting CypA with CsA reduces infection through TRIM5 activity, but only in cells that make TRIM5. On the other hand, CsA also inhibits IFITM3 which increases HIV infectivity in cells that make IFITM3. Thus, the effect of CsA is dependent on the levels of TRIM5 and IFITM3. High TRIM5, low IFITM3, CsA inhibits infection. Low TRIM5, high IFITM3 CsA rescues infection (IFN treated THP-1 are high IFITM3, low TRIM5 so they allow one to isolate the effect of drugs on IFITM3). Most cells make both proteins to some degree. This is why CsA is not an effective transduction enhancer and also why the results differ between lines, depending on how much CypA and IFITM3 they make. Stem cells make TRIM5 so one cannot use CsA. Thus, in the present work it has been sought to develop molecules that inhibit IFITM3 and not CypA, such as BG147.

### Further Studies

THP-1 cells (human monocytic cell line) were treated with 10 ng/ml type 1 interferon (IFNβ) overnight to induce IFITM3 expression.

The following day IFN treated (closed symbols) and untreated cells (open symbols) were infected with VSV-G pseudotyped HIV-1 vector encoding GFP (HIV-GFP) at a multiplicity of infection (MOI) of 0.4. Cells were infected in the presence of a titration of either BG147 or Cyclosporine H (CsH). 48 hours later infection levels were determined by counting GFP positive green cells by flow cytometry.

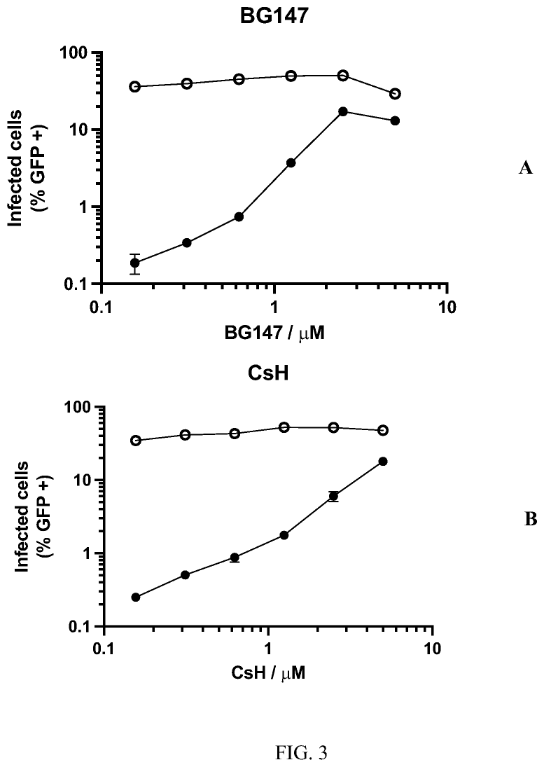

Results are shown in FIG. 3. In each of panels A and B, results for IFN treated cells are shown as closed symbols, and results for untreated cells as open symbols. Panel A shows results with BG147 and panel B shows results with CsH.

Both BG147 and CsH rescue infection from the antiviral effect of IFN. BG147 is more potent than CsH.

### Example 5—Stem Cell Studies

Here inhibitors were tested for their ability to improve transduction of HIV vector in the primary human stem cells that are used for gene therapy. This is a gold standard test because it tests the ability to improve transduction in the same cells that are actually used for gene therapy.

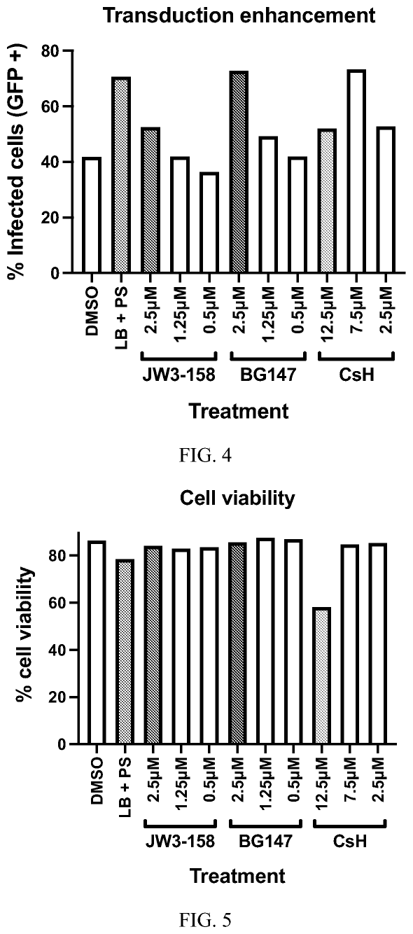

Human stem cells (HSC) were infected with a dose of VSV-G pseudotyped HIV gene therapy vector encoding GFP that infects 40% of the cells. Cells were infected in the presence and absence of various transduction enhancers as follows: Lentiboost (LB), Protamine Sulphate (PS), JW3-158, BG147 or CsH at concentrations of 2.5, 1.25 and 0.5 μM. 48 hours post transduction infected (green) cells were enumerated by flow cytometry. Results are shown in FIG. 4.

BG147 out performs other inhibitors and is as effective as state of the art transduction enhancer (combination of LB+PS). It was noted that competitor molecule CsH achieves good enhancement but at higher concentrations and is toxic at 12.5 μM.

This first experiment established effective concentration ranges.

Cell viability measurements from the cells in the above experiment showed that viability is good after BG147 and JW3-158 treatment and better than after PS+LB treatment (FIG. 5). Note that CsH is toxic at 12.5 μM and this is reflected in reduced transduction.

This experiment was then repeated as follows in cells from a second HSC donor.

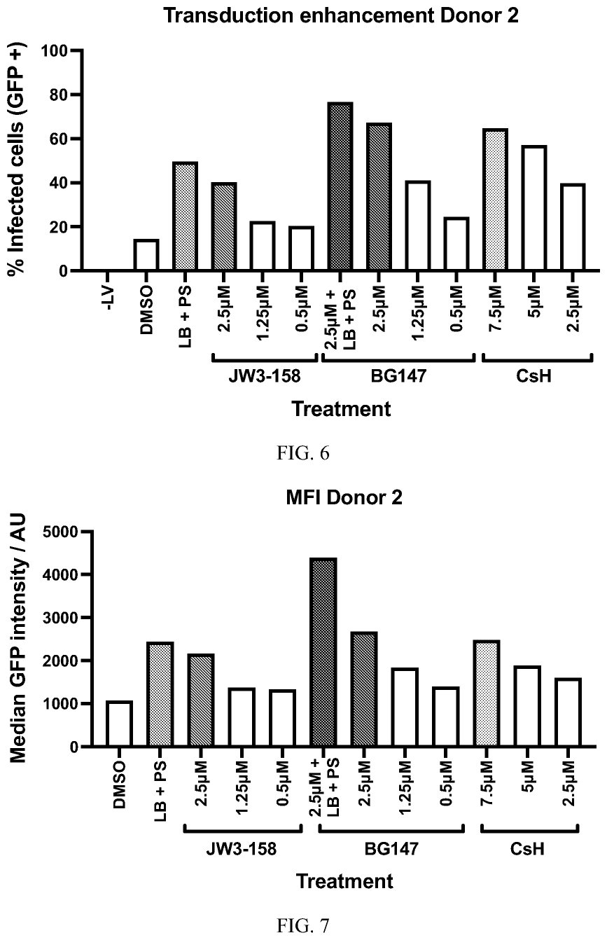

HSC were infected with a dose of VSV-G pseudotyped HIV gene therapy vector encoding GFP that infects 40% of the cells. Cells were infected in the presence and absence of various transduction enhancers as follows: Lentiboost (LB), Protamine Sulphate (PS), and JW3-158, BG147 or CsH at concentrations of 2.5, 1.25 and 0.5 μM. 48 hours post transduction infected (green) cells are enumerated by flow cytometry. Results are shown in FIG. 6

In this experiment BG147 out-performed other inhibitors including state of the art transduction enhancer combination of LB+PS.

Importantly, BG147 at 2.5 μM is additive with PS and LB giving the best number of transduced (infected) cells. This is expected because PS and LB improve the virus sticking to the cells whereas BG147 improves the process of viral entry, so they work well together.

### Protocol

Primary CD34+ HSCs were cultured in Stemspan II supplemented with 1% Pen/Strep, 100 ng/mL TPO, SCF and FLT3R, and 60 ng/mL IL-3. Cells were resuspended after thawing at a concentration of 10\hat{ }6/mL and allowed to recover for 24 h. On Day 2, cells were resuspended and incubated with or without GFP-Lentivirus (MOI 10), Lentiboost+Protamine Sulphate, JW3-158, BG-147 and CsH at various concentrations and combinations. The final volume was 125 uL in all cases with 250000 cells per condition.

The cells were incubated a further 48 h to allow GFP expression. Cells were then transferred to FACS tubes and centrifuged at 300×g for 10 minutes. Supernatant was aspirated before resuspension in 1 mL FACS buffer and centrifuging again to wash. Cells were then resuspended in 400 uL FACS buffer with DAPI. Viability and GFP expression were measured by flow cytometry.

### Example 6—Further Stem Cell Studies

Here a different measurement was made of transgene expression using the cells from the same experiment as that above (Example 5). Rather than measuring the number of cells transduced (% infection) the mean fluorescent intensity (greenness of the transgene expressing cells) was measured. Results are shown in FIG. 7.

This shows that the greenest cells are in the treatment with 2.5 μM BG147+PS+LB. This suggests that the infection after this treatment is the highest of all and the cells are greener because they have been infected with more than one HIV GFP virus. This also suggests that the maximum number of infected cells was achieved, i.e. the remaining 20% cannot be infected with HIV GFP whatever the dose.

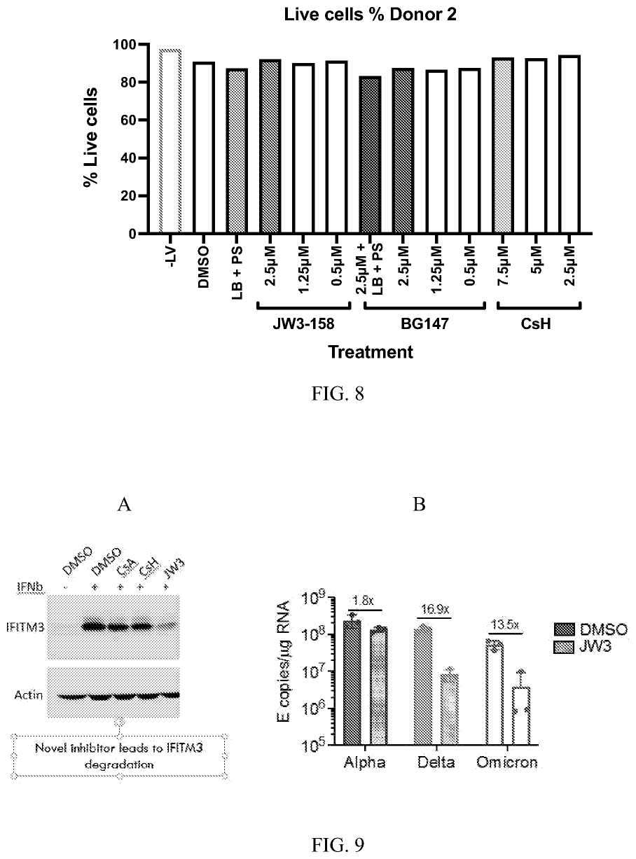

FIG. 8 summarises the live/dead stain data for the previous experiment showing that none of the treatments significantly killed the cells.

BG147 performs best. Without being limited by theory, it is proposed that JW3-158 is not working so well because it retains anti CypA activity that would optimally be removed or at least further reduced for the molecule to perform optimally in stem cells.

### Example 7—Antiviral Activity Against COVID-19

### Western Blot

THP-1 cells (human monocytic cell line) were treated with 10 ng/ul Type I interferon (IFNβ) overnight to induce IFITM3 expression. The following day the cells were treated with CsA, CsH or JW3 (JW3-158) at 5 μM or equivalent volume of DMSO vehicle. Protein samples were harvested 6 hours later and the IFITM3 levels measured by immunoblot with Actin detected as a loading control. IFN induces IFITM3. Results are shown in FIG. 9, panel A. Inhibitors result in loss of IFITM3 protein levels compared to DMSO treated cells (second lane), JW3-158 has the most potent effect

Calu-3 lung epithelial cells were pre-treated with either 2.5 μM JW3 (JW3-158) or an equivalent volume of DMSO for 4 h prior to infection with either Alpha, Delta or Omicron isolates of SARS-COV-2 at an MOI of 1000 viral E RNA copies/cell. The compound was present throughout infection. RNA was harvested at 48 hours post infection, and the level of viral replication quantified by RT-qPCR measuring E copies. Results are shown in FIG. 9, panel B. The fold change between DMSO and JW3-158 treated conditions is shown for each variant, where JW3-158 inhibits Delta and Omicron replication to a greater extent than Alpha.

### Example 8—Further Synthesis Studies

Additional work was directed to: (a) synthesis of a larger batch of BG147; and (b) synthesis of further compounds of the disclosure.

### Procedure Method

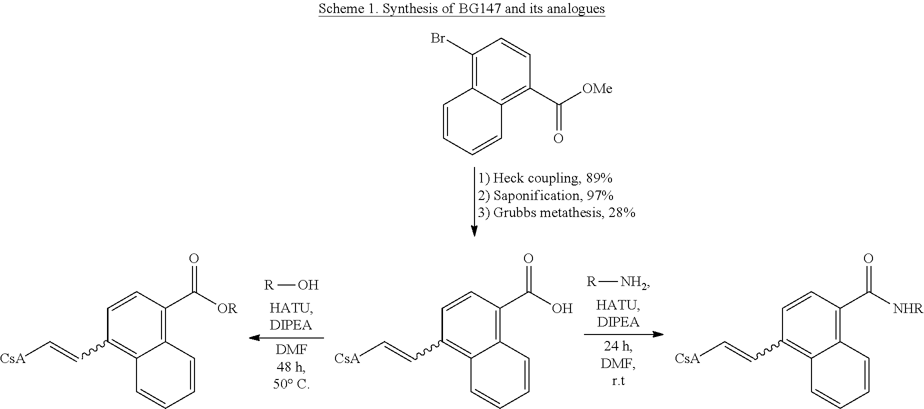

### Synthesis and Characterization of BG147 and its Analogues

### Methyl 4-vinyl-1-naphthoate (VP43)

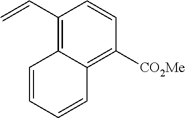 [SMILES: C=Cc1ccc(C(=O)OC)c2ccccc12]

Methyl 4-bromonaphthalene-1-carboxylate (530 mg, 2 mmol) was dissolved in THF:Water=9:1 (28 mL) followed by potassium vinyltrifluoroborate (1.3 g, 10 mmol), Cesium carbonate (1.9 g, 6 mmol), and [1,1′-Bis(diphenylphosphino) ferrocene]dichloropalladium (II) (1:1) (5 mol %). After stirring overnight at 70° C., the reaction mixture was cooled and condensed in vacuo. The residue was diluted with CH₂Cl₂ (10 mL), washed with water (3×10 mL) and brine, dried over MgSO₄ and concentrated under reduced pressure. The crude product was purified by chromatography column on silica gel (EtOAc 0%→100% in Cyclohexane) to afford VP43 (378 mg, 1.779 mmol, 89%) as a yellow oil.

### 4-vinyl-1-naphthoic acid (VP57)

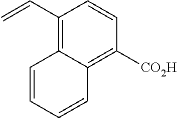

VP43 (2.2 g, 10 mmol) was dissolved in THF (12 mL) and treated with a solution 5M of LiOH (25 mL). After stirring overnight at 50° C., THF removed under vacuum and the residue was acidified with HCl 1M up to pH 2, extracted with EtOAc (30×3), dried over MgSO₄, filtered and concentrated under reduced pressure to afford VP57 (2 g, 10 mmol, 97%) as a yellow solid.

### 4-((4R,5R)-5-((2S,5S,11S,14S,17S,20S,23R,26S,29S,32S)-5-ethyl-11,17,26,29-tetraisobutyl-14,32-diisopropyl-1,7,10,16,20,23,25,28,31-nonamethyl-3,6,9,12,15,18,21,24,27,30,33-undecaoxo-1,4,7,10,13,16,19,22,25,28,31-undecaazacyclotritriacontan-2-yl)-5-hydroxy-4-methylpent-1-en-1-yl)-1-naphthoic acid (VP47)

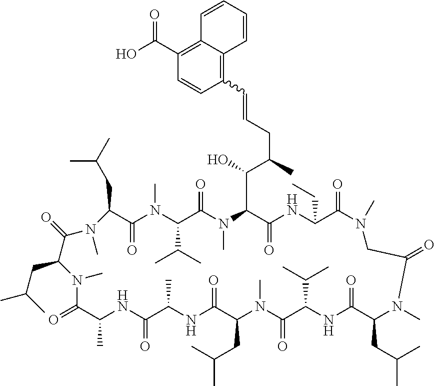 [SMILES: CC[C@@H]1NC(=O)[C@H]([C@H](O)[C@H](C)CC=Cc2ccc(C(=O)O)c3ccccc23)N(C)C(=O)[C@H](C(C)C)N(C)C(=O)[C@H](CC(C)C)N(C)C(=O)[C@H](CC(C)C)N(C)C(=O)[C@@H](C)NC(=O)[C@H](C)NC(=O)[C@H](CC(C)C)N(C)C(=O)[C@H](C(C)C)NC(=O)[C@H](CC(C)C)N(C)C(=O)CN(C)C1=O]

VP57 (270 mg, 1.36 mmol) was dissolved into anhydrous DCE (4.5 mL) followed by Cyclosporine A (360 mg, 0.299 mmol) and Hoveyda-Grubbs 2^{nd} generation catalyst (15 mol %). The reaction mixture was stirred under N₂ atmosphere in a MW reactor (70° C., 30 min) and then cooled to r.t. The solvent was removed under reduced pressure and the row material was redissolved in MeOH, passed through a Stratospheres PL Thiol MP SPE cartridge (polymer Lab, Varian Inc) to remove the catalyst. The unreacted CsA was removed by Biotage Isolute PE-AX 10 g (MeOH+10% NH₄OH→100% MeOH→5% MeOH in DCM→5% MeOH+5% AcOH in DCM). The excess of VP57 was removed by reverse phase chromatography (C18, MeCN 10%→100% in H₂O+10 mM NH₃) obtaining VP47 (115 mg, 0.085 mmol, 28%, trans/cis=3/1) as an off-white solid.

¹H-NMR (500 MHz, CD13, δ ppm) δ 3.56 (s, NMe, 3H), 3.40 (s, NMe, 3H), 3.27 (s, NMe, 3H), 3.11 (s, NMe, 3H), 3.10 (s, NMe, 3H), 2.72 (s, NMe, 3H), 2.67 (s, NMe, 3H).

¹³C-NMR (500 MHz, CDCl₃, δ ppm) δ 173.92 (C=O), 173.89 (C=O), 173.66 (C=O), 171.71 (C=O), 171.50 (C=O), 171.38 (C=O), 171.34 (C=O), 170.63 (C=O), 170.56 (C=O), 170.45 (C=O), 170.37 (C=O).

LCMS (m/z): [MH]⁺ calcd. for C₇₂H₁₁₅N₁₁O₁₄, 1358.9; found 1358.4

### General Procedure for the Synthesis of BG147 and its Ester and Amide Analogues

To a solution of VP47 (1 eq) in DMF (1 mL) were added DIPEA (7 eq) and HATU (6 eq). After stirring for 10 min at r.t., a solution of the appropriate alcohol or amine (10 eq) in DMF (0.5 mL) was added and the reaction mixture was stirred overnight at 50° C. Then, the solvent was removed under reduced pressure and the crude product was purified by chromatography column on silica gel (MeOH 0%→10% in DCM) and by reverse phase C4 (MeCN 30%→100% in H₂O 10 mM NH₃) obtaining the final product.

### 2-(1H-imidazol-1-yl)ethyl 4-((4R,5R)-5-((2S,5S,11S,14S,17S,20S,23R,26S,29S,32S)-5-ethyl-11,17,26,29-tetraisobutyl-14,32-diisopropyl-1,7,10,16,20,23,25,28,31-nonamethyl-3,6,9,12,15,18,21,24,27,30,33-undecaoxo-1,4,7,10,13,16,19,22,25,28,31-undecaazacyclotritriacontan-2-yl)-5-hydroxy-4-methylpent-1-en-1-yl)-1-naphthoate (BG147)

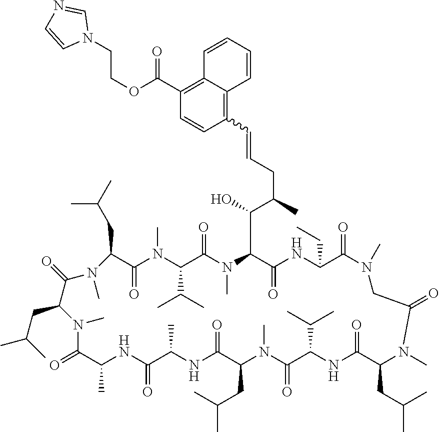 [SMILES: CC[C@@H]1NC(=O)[C@H]([C@H](O)[C@H](C)CC=Cc2ccc(C(=O)OCCn3ccnc3)c3ccccc23)N(C)C(=O)[C@H](C(C)C)N(C)C(=O)[C@H](CC(C)C)N(C)C(=O)[C@H](CC(C)C)N(C)C(=O)[C@@H](C)NC(=O)[C@H](C)NC(=O)[C@H](CC(C)C)N(C)C(=O)[C@H](C(C)C)NC(=O)[C@H](CC(C)C)N(C)C(=O)CN(C)C1=O]

BG147 was obtained as an off-white solid (17 mg, 0.01 mmol, 20%, trans/cis=3/1) following the general procedure described above and using 2-(1H-imidazol-1-yl) ethan-1-ol as appropriate alcohol.

¹H-NMR (600 MHz, CDl3, δ ppm) δ 3.55 (s, NMe, 3H), 3.39 (s, NMe, 3H), 3.27 (s, NMe, 3H), 3.10 (s, NMe, 3H), 3.09 (s, NMe, 3H), 2.70 (s, NMe, 3H), 2.66 (s, NMe, 3H).

¹³C-NMR (600 MHz, CDCl₃, δ ppm) δ 173.97 (C=O), 173.88 (C=O), 173.63 (C=O), 173.13 (C=O), 172.80 (C=O), 172.16 (C=O), 171.54 (C=O), 171.43 (C=O), 171.26 (C=O), 170.53 (C=O), 170.28 (C=O).

HRMS (m/z): [MH]⁺ calcd. for C₇₇H₁₂₁N₁₃O₁₄, 1452.91560; found 1452.92287

### 2-morpholinoethyl 4-((4R,5R)-5-((2S,5S,11S,14S,17S,20S,23R,26S,29S,32S)-5-ethyl-11,17,26,29-tetraisobutyl-14,32-diisopropyl-1,7,10,16,20,23,25,28,31-nonamethyl-3,6,9,12,15,18,21,24,27,30,33-undecaoxo-1,4,7,10,13,16,19,22,25,28,31-undecaazacyclotritriacontan-2-yl)-5-hydroxy-4-methylpent-1-en-1-yl)-1-naphthoate

(VP50)

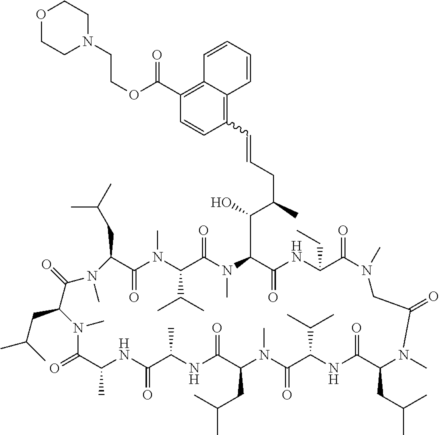 [SMILES: CC[C@@H]1NC(=O)[C@H]([C@H](O)[C@H](C)CC=Cc2ccc(C(=O)OCCN3CCOCC3)c3ccccc23)N(C)C(=O)[C@H](C(C)C)N(C)C(=O)[C@H](CC(C)C)N(C)C(=O)[C@H](CC(C)C)N(C)C(=O)[C@@H](C)NC(=O)[C@H](C)NC(=O)[C@H](CC(C)C)N(C)C(=O)[C@H](C(C)C)NC(=O)[C@H](CC(C)C)N(C)C(=O)CN(C)C1=O]

VP50 was obtained as an off-white solid (3.0 mg, 0.002 mmol, 17%, trans/cis=3/1) following the general procedure described above and using 2-morpholinoethan-1-ol as appropriate alcohol.

¹H-NMR (600 MHz, CDCl₃, δ ppm) δ 3.56 (s, NMe, 3H), 3.41 (s, NMe, 3H), 3.28 (s, NMe, 3H), 3.11 (s, NMe, 3H), 3.10 (s, NMe, 3H), 2.70 (s, NMe, 3H), 2.66 (s, NMe, 3H). LCMS (m/z): [MH]⁺ calcd. for C₇₈H₁₂₆N₁₂O₁₅, 1471.9; found 1471.6

### (3S,6S,9S,12R,15S,18S,21S,24S,30S,33S)-30-ethyl-33-((1R,2R)-1-hydroxy-5-(4-(4-(2-hydroxyethyl) piperazine-1-carbonyl) naphthalen-1-yl)-2-methylpent-4-en-1-yl)-6,9,18,24-tetraisobutyl-3,21-diisopropyl-1,4,7,10,12,15,19,25,28-nonamethyl-1,4,7,10,13,16,19,22,25,28,31-undecaazacyclotritriacontan-2,5,8,11,14,17,20,23,26,29,32-undecaone (VP51)

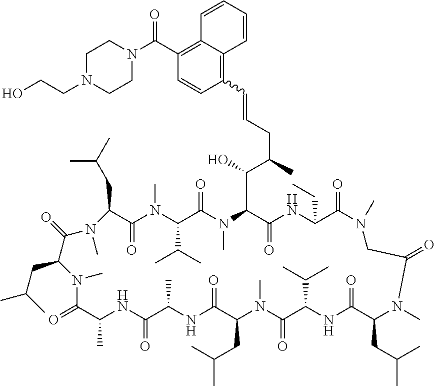 [SMILES: CC[C@@H]1NC(=O)[C@H]([C@H](O)[C@H](C)CC=Cc2ccc(C(=O)N3CCN(CCO)CC3)c3ccccc23)N(C)C(=O)[C@H](C(C)C)N(C)C(=O)[C@H](CC(C)C)N(C)C(=O)[C@H](CC(C)C)N(C)C(=O)[C@@H](C)NC(=O)[C@H](C)NC(=O)[C@H](CC(C)C)N(C)C(=O)[C@H](C(C)C)NC(=O)[C@H](CC(C)C)N(C)C(=O)CN(C)C1=O]

VP51 was obtained as an off-white solid (3.0 mg, 0.002 mmol, 18%, trans/cis=3/1) following the general procedure described above and using 2-(piperazin-1-yl) ethan-1-ol as appropriate amine.

¹H-NMR (600 MHz, CDCl₃, δ ppm) δ 3.55 (s, NMe, 3H), 3.41 (s, NMe, 3H), 3.27 (s, NMe, 3H), 3.12 (s, NMe, 3H), 3.00 (s, NMe, 3H), 2.71 (s, NMe, 3H), 2.67 (s, NMe, 3H). LCMS (m/z): [MH]⁺ calcd. for C₇₈H₁₂₇N₁₃O₁₄, 1,471.0; found 1471.2

### N-(2-(1H-imidazol-1-yl)ethyl)-4-((4R,5R)-5-((25,5S,11S,14S,17S,20S,23R,26S,29S,32S)-5-ethyl-11,17,26,29-tetraisobutyl-14,32-diisopropyl-1,7,10,16,20,23,25,28,31-nonamethyl-3,6,9,12,15,18,21,24,27,30,33-undecaoxo-1,4,7,10,13,16,19,22,25,28,31-undecaazacyclotritriacontan-2-yl)-5-hydroxy-4-methylpent-1-en-1-yl)-1-naphthamide (VP53)

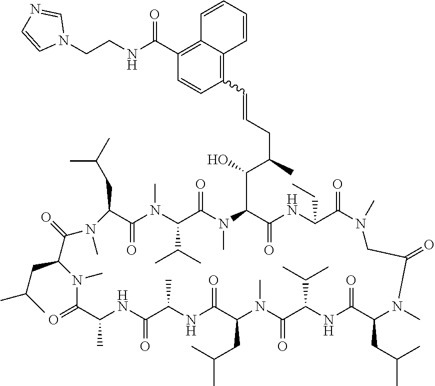 [SMILES: CC[C@@H]1NC(=O)[C@H]([C@H](O)[C@H](C)CC=Cc2ccc(C(=O)NCCn3ccnc3)c3ccccc23)N(C)C(=O)[C@H](C(C)C)N(C)C(=O)[C@H](CC(C)C)N(C)C(=O)[C@H](CC(C)C)N(C)C(=O)[C@@H](C)NC(=O)[C@H](C)NC(=O)[C@H](CC(C)C)N(C)C(=O)[C@H](C(C)C)NC(=O)[C@H](CC(C)C)N(C)C(=O)CN(C)C1=O]

VP53 was obtained as an yellow solid (8.0 mg, 0.006 mmol, 47%, trans/cis-3/1) following the general procedure described above and using 2-(1H-imidazol-1-yl) ethan-1-amine as appropriate amine.

¹H-NMR (600 MHz, CDCl₃, δ ppm) δ 3.55 (s, NMe, 3H), 3.37 (s, NMe, 3H), 3.29 (s, NMe, 3H), 3.08 (s, NMe, 3H), 3.03 (s, NMe, 3H), 2.71 (s, NMe, 3H), 2.67 (s, NMe, 3H).

LCMS (m/z): [MH]⁺ calcd. for C₇₇H₁₂₂N₁₄O₁₃, 1451.9; found 1452.2

### 2-(1H-pyrazol-1-yl)ethyl 4-((4R,5R)-5-((2S,5S,11S,14S,17S,20S,23R,26S,29S,32S)-5-ethyl-11,17,26,29-tetraisobutyl-14,32-diisopropyl-1,7,10,16,20,23,25,28,31-nonamethyl-3,6,9,12,15,18,21,24,27,30,33-undecaoxo-1,4,7,10,13,16,19,22,25,28,31-undecaazacyclotritriacontan-2-yl)-5-hydroxy-4-methylpent-1-en-1-yl)-1-naphthoate (VP72)

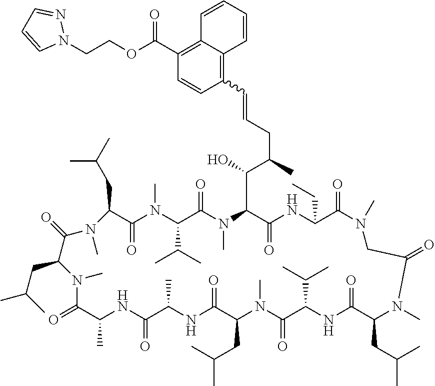 [SMILES: CC[C@@H]1NC(=O)[C@H]([C@H](O)[C@H](C)CC=Cc2ccc(C(=O)OCCn3cccn3)c3ccccc23)N(C)C(=O)[C@H](C(C)C)N(C)C(=O)[C@H](CC(C)C)N(C)C(=O)[C@H](CC(C)C)N(C)C(=O)[C@@H](C)NC(=O)[C@H](C)NC(=O)[C@H](CC(C)C)N(C)C(=O)[C@H](C(C)C)NC(=O)[C@H](CC(C)C)N(C)C(=O)CN(C)C1=O]

VP72 was obtained as an yellow solid (3.0 mg, 0.002 mmol, 14%, trans/cis=3/1) following the general procedure described above and using 2-(1H-pyrazol-1-yl) ethan-1-ol as appropriate alcohol.

¹H-NMR (500 MHz, CDCl₃, δ ppm) δ 3.56 (s, NMe, 3H), 3.41 (s, NMe, 3H), 3.27 (s, NMe, 3H), 3.11 (s, NMe, 3H), 3.10 (s, NMe, 3H), 2.71 (s, NMe, 3H), 2.67 (s, NMe, 3H).

LCMS (m/z): [MH]⁺ calcd. for C₇₇H₁₂₁N₁₃O₁₄, 1451.9; found 1453.0

### 3-(1H-pyrazol-1-yl) propyl 4-((4R,5R)-5-((2S,5S,11S,14S,17S,20S,23R,26S,29S,32S)-5-ethyl-11,17,26,29-tetraisobutyl-14,32-diisopropyl-1,7,10,16,20,23,25,28,31-nonamethyl-3,6,9,12,15,18,21,24,27,30,33-undecaoxo-1,4,7,10,13,16,19,22,25,28,31-undecaazacyclotritriacontan-2-yl)-5-hydroxy-4-methylpent-1-en-1-yl)-1-naphthoate (VP73)

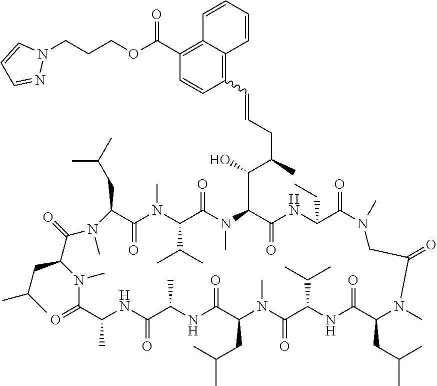 [SMILES: CC[C@@H]1NC(=O)[C@H]([C@H](O)[C@H](C)CC=Cc2ccc(C(=O)OCCCn3cccn3)c3ccccc23)N(C)C(=O)[C@H](C(C)C)N(C)C(=O)[C@H](CC(C)C)N(C)C(=O)[C@H](CC(C)C)N(C)C(=O)[C@@H](C)NC(=O)[C@H](C)NC(=O)[C@H](CC(C)C)N(C)C(=O)[C@H](C(C)C)NC(=O)[C@H](CC(C)C)N(C)C(=O)CN(C)C1=O]

VP73 was obtained as an yellow solid (3.0 mg, 0.002 mmol, 14%, trans/cis=3/1) following the general procedure described above and using 3-(1H-pyrazol-1-yl) propan-1-ol as appropriate alcohol.

¹H-NMR (500 MHz, CDCl₃, δ ppm) δ 3.56 (s, NMe, 3H), 3.41 (s, NMe, 3H), 3.28 (s, NMe, 3H), 3.11 (s, NMe, 3H), 3.10 (s, NMe, 3H), 2.71 (s, NMe, 3H), 2.67 (s, NMe, 3H).

LCMS (m/z): [MH]⁺ calcd. for C₇₈H₁₂₃N₁₃O₁₄, 1465.9; found 1467.1

### 4-(1H-imidazol-1-yl)butyl 4-((4R,5R)-5-((2S,5S,11S,14S,17S,20S,23R,26S,29S,32S)-5-ethyl-11,17,26,29-tetraisobutyl-14,32-diisopropyl-1,7,10,16,20,23,25,28,31-nonamethyl-3,6,9,12,15,18,21,24,27,30,33-undecaoxo-1,4,7,10,13,16,19,22,25,28,31-undecaazacyclotritriacontan-2-yl)-5-hydroxy-4-methylpent-1-en-1-yl)-1-naphthoate (VP74)

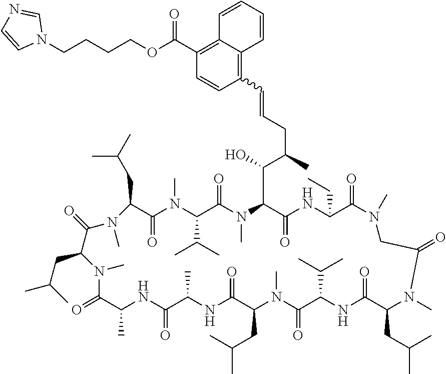 [SMILES: CC[C@@H]1NC(=O)[C@H]([C@H](O)[C@H](C)CC=Cc2ccc(C(=O)OCCCCn3ccnc3)c3ccccc23)N(C)C(=O)[C@H](C(C)C)N(C)C(=O)[C@H](CC(C)C)N(C)C(=O)[C@H](CC(C)C)N(C)C(=O)[C@@H](C)NC(=O)[C@H](C)NC(=O)[C@H](CC(C)C)N(C)C(=O)[C@H](C(C)C)NC(=O)[C@H](CC(C)C)N(C)C(=O)CN(C)C1=O]

VP74 was obtained as an yellow solid (4.0 mg, 0.003 mmol, 18%, trans/cis=1/1) following the general procedure described above and using 4-(1H-imidazol-1-yl) butan-1-ol as appropriate alcohol.

¹H-NMR (500 MHz, CDCl₃, δ ppm) δ 3.55 (s, NMe, 3H), 3.40 (s, NMe, 3H), 3.28 (s, NMe, 3H), 3.11 (s, NMe, 3H), 3.10 (s, NMe, 3H), 2.71 (s, NMe, 3H), 2.67 (s, NMe, 3H).

LCMS (m/z): [MH]⁺ calcd. for C₇₉H₁₂₅N₁₃O₁₄, 1479.9; found 1481.1

### Methyl 4-((4R,5R)-5-((2S,5S,11S,14S,17S,20S,23R,26S,29S,32S)-5-ethyl-11,17,26,29-tetraisobutyl-14,32-diisopropyl-1,7,10,16,20,23,25,28,31-nonamethyl-3,6,9,12,15,18,21,24,27,30,33-undecaoxo-1,4,7,10,13,16,19,22,25,28,31-undecaazacyclotritriacontan-2-yl)-5-hydroxy-4-methylpent-1-en-1-yl)-1-naphthoate (VP75)

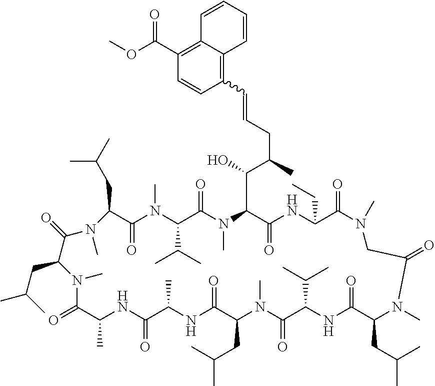 [SMILES: CC[C@@H]1NC(=O)[C@H]([C@H](O)[C@H](C)CC=Cc2ccc(C(=O)OC)c3ccccc23)N(C)C(=O)[C@H](C(C)C)N(C)C(=O)[C@H](CC(C)C)N(C)C(=O)[C@H](CC(C)C)N(C)C(=O)[C@@H](C)NC(=O)[C@H](C)NC(=O)[C@H](CC(C)C)N(C)C(=O)[C@H](C(C)C)NC(=O)[C@H](CC(C)C)N(C)C(=O)CN(C)C1=O]

VP75 was obtained as an yellow solid (11.0 mg, 0.008 mmol, 54%, trans/cis=3/1) following the general procedure described above and using methanol as appropriate alcohol.

¹H-NMR (500 MHz, CDCl₃, δ ppm) δ 3.56 (s, NMe, 3H), 3.40 (s, NMe, 3H), 3.27 (s, NMe, 3H), 3.11 (s, NMe, 3H), 3.10 (s, NMe, 3H), 2.71 (s, NMe, 3H), 2.67 (s, NMe, 3H).

LCMS (m/z): [MH]⁺ calcd. for C₇₃H₁₁₇N₁₁O₁₄, 1371.9; found 1373.0

### 4-((4R,5R)-5-((2S,5S,11S,14S,17S,20S,23R,26S,29S,32S)-5-ethyl-11,17,26,29-tetraisobutyl-14,32-diisopropyl-1,7,10,16,20,23,25,28,31-nonamethyl-3,6,9,12,15,18,21,24,27,30,33-undecaoxo-1,4,7,10,13,16,19,22,25,28,31-undecaazacyclotritriacontan-2-yl)-5-hydroxy-4-methylpent-1-en-1-yl)-N-(2-morpholinoethyl)-1-naphthamide (VP76)

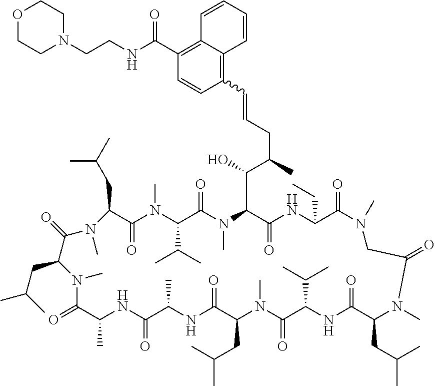 [SMILES: CC[C@@H]1NC(=O)[C@H]([C@H](O)[C@H](C)CC=Cc2ccc(C(=O)NCCN3CCOCC3)c3ccccc23)N(C)C(=O)[C@H](C(C)C)N(C)C(=O)[C@H](CC(C)C)N(C)C(=O)[C@H](CC(C)C)N(C)C(=O)[C@@H](C)NC(=O)[C@H](C)NC(=O)[C@H](CC(C)C)N(C)C(=O)[C@H](C(C)C)NC(=O)[C@H](CC(C)C)N(C)C(=O)CN(C)C1=O]

VP76 was obtained as an white solid (10.0 mg, 0.007 mmol, 46%, trans/cis-3/1) following the general procedure described above and using 2-morpholinoethan-1-amine as appropriate amine.

¹H-NMR (500 MHz, CDCl₃, δ ppm) δ 3.55 (s, NMe, 3H), 3.38 (s, NMe, 3H), 3.27 (s, NMe, 3H), 3.10 (s, NMe, 3H), 3.08 (s, NMe, 3H), 2.72 (s, NMe, 3H), 2.67 (s, NMe, 3H).

LCMS (m/z): [MH]⁺ calcd. for C₇₈H₁₂₇N₁₃O₁₄, 1470.0; found 1471.3

### Materials and Methods

All commercially available solvents and reagents were used without further treatment as received unless otherwise noted.
Solvent evaporations were performed under reduced pressure (40-60° C.) by using Buchi Rotavapor R-210.
Microwave-assisted reactions were carried out in Biotage Initiator⁺ apparatus for synthesis.
Reactions under anhydrous atmosphere were performed using commercial N₂.
Thin layer chromatography (TLC) was performed with qualitative purposes on aluminium silica gel plates (Alugram Sil G/UV 254) with detection by UV light (λ 254 nm) and by staining with [(NH₄)₆MoO₄, Ce(SO₄)₂, H₂SO₄, H₂O].
Purifications were carried in a Biotage Isolera Four Flash Chromatography System. The chromatography column used were: a) normal phase Biotage Sfär Silica (5 g, 10 g, 25 g), and b) reverse phase Biotage Snap Bio C4 300 Å 25 g or Biotage Sfar C18 D-Duo 100 Å (12 g, 30 g, 60 g). PE-AX 10 g, strong base ion exchange resin was used when indicated.
¹H- and ¹³C-NMR spectra were performed at UCL Chemistry NMR Facility and Bruker DRX 500, 600 or 700 MHz spectrometer were used. Chemical shifts (8) are expressed in ppm relative to TMS as an internal standard and coupling constants (J) in Hz. J are assigned and not repeated. CDCl₃ and CD₃OD were used as solvents at room temperature except when indicated. All the assignments were confirmed by 2D spectra (COSY and HSCQ).
Accurate mass measurements using an ASAP-HESI ionisation connected to the Q Exactive Plus mass spectrometer were performed at UCL Chemistry Mass Spectrometry Facility.
LC-MS spectra were obtained using a single quadrupole LC/MSD XT mass spectrometer with electrospray ionisation (ESI), using an analytical C4 column (Symmetry 300, 50× 4.6 mm, 3.5 μm) and C18 column (Kinetex 5 μm 100 Å, 50×4.6 mm). The gradients used were:
   a) 10%→95% of MeCN+0.1% FA (FA=formic acid) in H₂O+0.1% FA (6.5 min)
b) 30%→95% of MeCN+0.1% FA in H₂O+0.1% FA (9 min)
c) 30%→95% of MeCN in H₂O 10 mM NH₃ (9 min)

### Example 9—Activity Studies on Further Compounds of the Disclosure

Further studies were undertaken to test the properties of additional compounds of the disclosure.

### Study 1:

THP-1 cells (human monocytic cell line) were treated with 10 ng/ml type 1 interferon (IFNβ) overnight to induce IFITM3 expression.

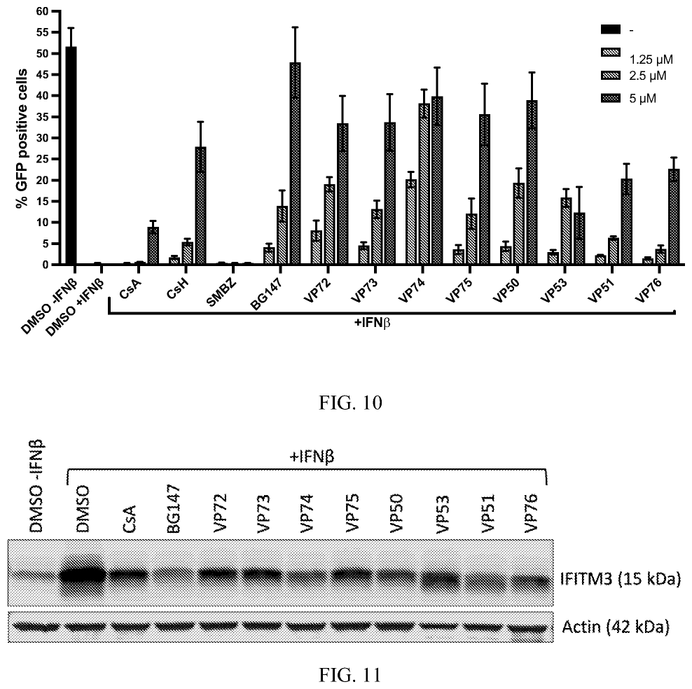

The following day IFN treated cells were infected with VSV-G pseudotyped HIV-1 vector encoding GFP (HIV-GFP) at a multiplicity of infection (MOI) of 0.25. Cells were infected in the presence of a titration (1.25-5 μM) of one of a range of control and comparative compounds, as indicated in FIG. 10. 48 hours later, cells were fixed and infection levels were determined by counting GFP positive cells by flow cytometry. For comparison, DMSO treated THP-1 cells with and without IFNβ, are shown by black bars.

Results are shown in FIG. 10 (N=2). A high level of % GFP positive cells at a particular test compound concentration indicates a high level of rescued infection from the antiviral effect of IFN.

### Study 2 (Western Blot):

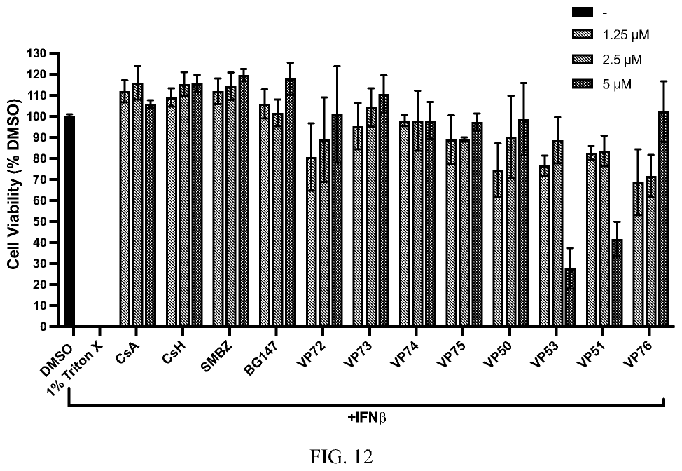

THP-1 cells (human monocytic cell line) were treated with 10 ng/ml type 1 interferon (IFNβ) overnight to induce IFITM3 expression. Cells were then treated with 5 μM of the specified test compounds. After 24 hrs cells were lysed and run on SDS PAGE gels. Western Blot analysis was performed using anti-Actin (control) and anti-IFITM3 antibodies to visualise IFITM3 degradation. Results are shown in FIG. 11.

### Study 3 (Cell Viability Assay (MTT)):

A MTT colourimetric viability assay of THP1 cells in the presence of the specified test compounds was performed in parallel with infection experiments. THP-1 cells (human monocytic cell line) were treated with 10 ng/ml type 1 interferon (IFNβ) overnight to induce IFITM3 expression. The following day, cells were treated with a titration (1.25-5 μM) of one of a range of control and comparative compounds, as indicated in FIG. 12. Triton X was a control to show cell death. After 48 hours, MTT reagent was added to treated cells, after 4 hours at 37° C. cells crystal metabolites were solubilised and a spectrometer used to measure absorbance at 570 nm. Results are shown in FIG. 12. Error bars represent the standard deviation of three technical replicates from one experiment. N=1. For each test compound, three bars are shown, which in each instance correspond, from left to right, to results obtained when using 1.25, 2.5 and 5 μM of test compound, respectively.

### Example 10-Synthesis of Intermediates Useful for Preparation of the Target Compounds

### VP_130

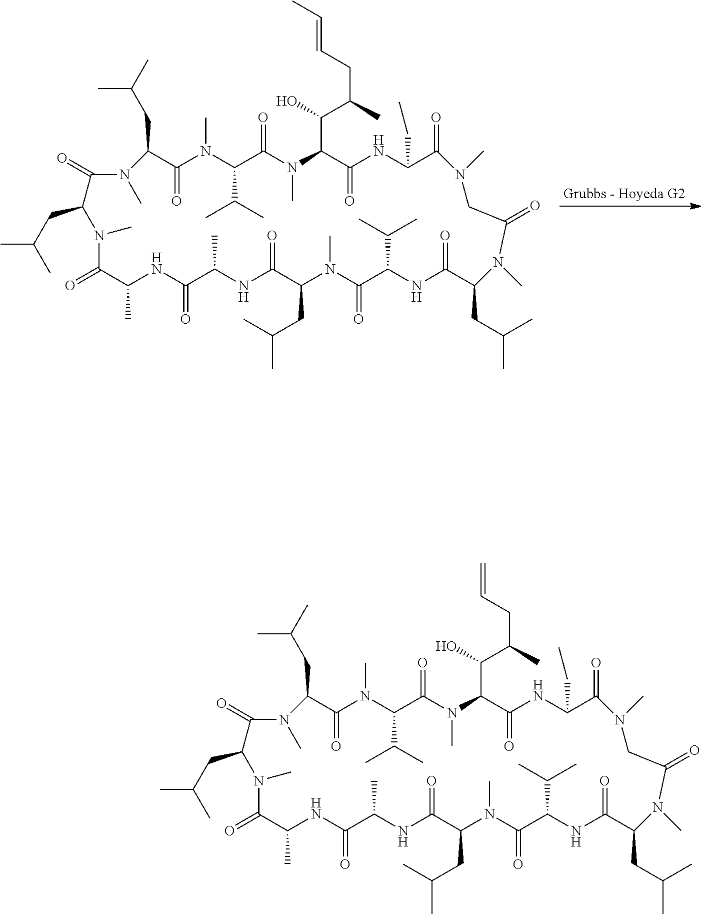 [SMILES: C/C=C/C[C@@H](C)[C@@H](O)[C@H]1C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C ; C=CC[C@@H](C)[C@@H](O)[C@H]1C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

CsA (500.0 mg 0.416 1 eq) was dissolved into DCE (10 mL) in a microwave vial followed by Grubbs'-Hoyveda G2 (18.3 mg 0.029 mmol) and sparged with Ethylene gas. The vial was irradiated at 70° C. for 30 minutes (MW). The reaction passed through SH resin column to remove catalyst. Rinsed with MeOH and concentrated. Crude 490 mg, 0.412 mmol, 92%). The product was used without further purification (pure by lcms, C4 Acetonitrile: Water: 0.1% Formic acid). [MH]+ 1189.

### VP_136

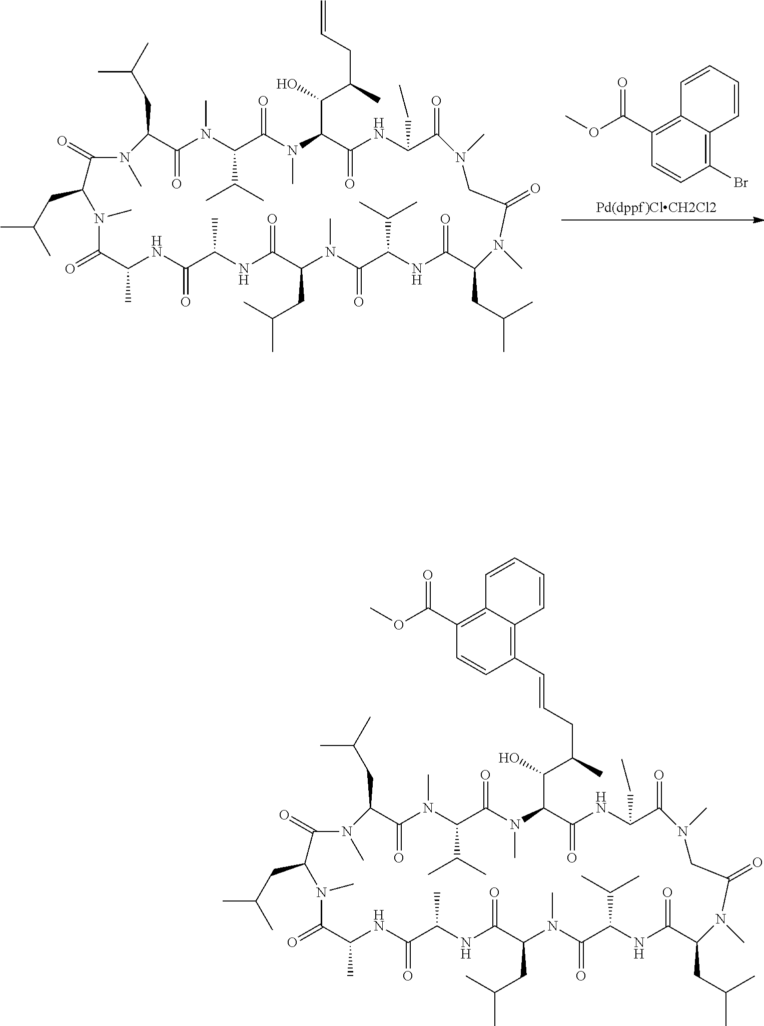 [SMILES: C=CC[C@@H](C)[C@@H](O)[C@H]1C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C ; COC(=O)c1ccc(Br)c2ccccc12 ; CC[C@@H]1NC(=O)[C@H]([C@H](O)[C@H](C)C/C=C/c2ccc(C(=O)OC)c3ccccc23)N(C)C(=O)[C@H](C(C)C)N(C)C(=O)[C@H](CC(C)C)N(C)C(=O)[C@H](CC(C)C)N(C)C(=O)[C@@H](C)NC(=O)[C@H](C)NC(=O)[C@H](CC(C)C)N(C)C(=O)[C@H](C(C)C)NC(=O)[C@H](CC(C)C)N(C)C(=O)CN(C)C1=O]

The bromide ((80.3 mg, 0.303 mmol, 5.000 eq) was dissolved in THF*:Water=9:1 (2 mL) followed by the VP130 ((72.0 mg, 0.061 mmol, 1.000 eq)), Cesium carbonate (59.2 mg, 0.182 mmol, 3.000 eq), and Pd(dppf)Cl·CH₂Cl₂ (2.5 mg, 0.003 mmol, 0.050 eq) (5 mol %). The mixture was stirred at 70° C. for 4 days in the microwave. The residue was partitioned between DCM (10 mL) and water (10 mL), the organic phase washed with Brine, dried over MgSO₄ and concentrated under reduced pressure. Purification using acetonitrile/water 0.1% formic acid, C4 column gave product (25 mg, 0.022 mmol, 30%). [MH]+ 1373.

The resulting methyl ester compound VP_136 can readily be converted into target compounds, including (but not limited to) by hydrolysis to the corresponding carboxylic acid, i.e. VP47 (e.g. with LiOH), followed by esterification with an appropriate alcohol (see, e.g., the representative examples in Example 8, showing analogous reactions utilising VP47).

1. A cyclosporine analogue that is a compound of formula (I): 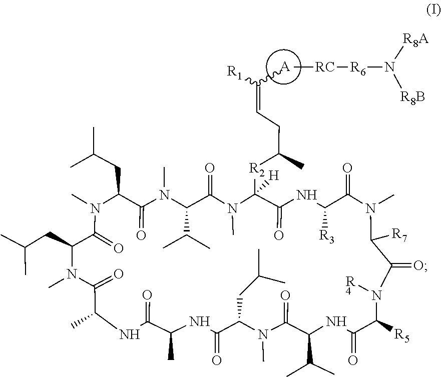
or a pharmaceutically acceptable salt thereof,
wherein:
R₁ represents hydrogen, C₁-C₄ alkyl or C₂-C₄ alkenyl;
R₂ represents 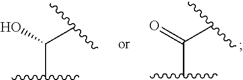
R₃ represents ethyl or isopropyl;
R₄ represents methyl or ethyl;
R₅ represents —CH₂CH(CH₃)₂, —CH₂CH(CH₃)CH₂CH₃, —CH(CH₃)CH₃, or —CH(CH₃)CH₂CH₃;
R₇ represents a hydrogen atom or a moiety that is a C₁₋₂₀ alkyl group, a C₂₋₂₀ alkenyl group or a C₂₋₂₀ alkynyl group, which moiety is unsubstituted or substituted by one or more substituents selected from halogen atoms and sulfonic acid groups, and in which
(a) 0, 1, 2 or 3 carbon atoms are replaced by groups selected from C₆₋₁₀ arylene, 5- to 10-membered heteroarylene, C₃₋₇ carbocyclylene and 5- to 10-membered heterocyclylene groups, and
(b) up to half of the —CH₂— groups are replaced by groups selected from —O—, —S—, —C(O)— and —N(C₁₋₆ alkyl)-groups, wherein:
(i) said arylene, heteroarylene, carbocyclylene and heterocyclylene groups are unsubstituted or substituted by one or more substituents selected from halogen atoms and C₁₋₆ alkyl, C₁₋₆ haloalkyl, C₁₋₆ alkoxy, —(C₁₋₆ alkyl)ₙC(O)O(C₁₋₆ alkyl) (where n=0 or 1), —(C₁₋₆ alkyl)ₙOC(O)(C₁₋₆ alkyl) (where n=0 or 1), C₁₋₆ alkylthiol, —N(R_{N})₂ (wherein each R_{N} independently represents a hydrogen atom or a C₁₋₆ alkyl group), —CN, —S(O)₂NH₂, nitro and sulfonic acid groups; and
(ii) 0, 1 or 2 carbon atoms in said carbocyclylene and heterocyclylene groups are replaced by —C(O)— groups; and
Ring A represents a monocyclic ring or bicyclic ring system, is a C₆₋₁₀ arylene group or a 5- to 10-membered heteroarylene group, and is unsubstituted or substituted by one or more substituents selected from halogen atoms and C₁₋₆ alkyl, C₁₋₆ haloalkyl, C₁₋₆ alkoxy, —(C₁₋₆ alkyl)ₙC(O)O(C₁₋₆ alkyl) (where n=0 or 1), —(C₁₋₆ alkyl)ₙOC(O)(C₁₋₆ alkyl) (where n=0 or 1), C₁₋₆ alkylthiol, —N(R_{N})₂ (wherein each R_{N} independently represents a hydrogen atom or a C₁₋₆ alkyl group), —CN, —S(O)₂NH₂, nitro and sulfonic acid groups;
and wherein the moiety —R_{C}—R₆—N(R_{8A})(R_{8B}) is defined according to either (X) or (Y):
(X):
R_{C} represents a moiety selected from the group consisting of —C(O)O—, —OC(O)—, —C(O)N(R_{N})—, —N(R_{N})C(O)—, —S(O)₂N(R_{N})—, —N(R_{N})S(O)₂—, —N(R_{N})—C(O)—N(R_{N})—, —N(R_{N})—C(S)—N(R_{N})—, —C(O)CH₂—, —CH₂C(O)—, —C(CF₃)N(R_{N})—, —N(R_{N})C(CF₃)—, —C(O)NF—, —NFC(O)—, —C(CN)═N—O—, —O—N═C(CN)—, —N(R_{N})C(O)O—, —OC(O)N(R_{N})—, phenylene, 5- to 6-membered heteroarylene, C₅₋₆ carbocyclylene and 5- to 6-membered heterocyclylene, wherein each R_{N} independently represents a hydrogen atom or a C₁₋₆ alkyl group, and wherein said phenylene, said heteroarylene, said carbocyclylene and said heterocyclylene are each unsubstituted or substituted by one or more substituents selected from halogen atoms and C₁₋₆ alkyl, C₁₋₆ haloalkyl, C₁₋₆ alkoxy, —(C₁₋₆ alkyl)ₙC(O)O(C₁₋₆ alkyl) (where n=0 or 1), —(C₁₋₆ alkyl)ₙOC(O)(C₁₋₆ alkyl) (where n=0 or 1), C₁₋₆ alkylthiol, —N(R_{N})₂ (wherein each R_{N} independently represents a hydrogen atom or a C₁₋₆ alkyl group), —CN, —S(O)₂NH₂, nitro and sulfonic acid groups;
R₆ represents a C₁₋₆ alkylene group, a C₂₋₆ alkenylene group or a C₂₋₆ alkynylene group, and is unsubstituted or substituted by one or more substituents selected from halogen atoms and sulfonic acid groups; and
R_{8A} and R_{8B} either:
(a) together with the nitrogen atom to which they are attached, form a 5- to 10-membered heteroaryl group or 5- to 10-membered heterocyclyl group, wherein said heteroaryl group and said heterocyclyl group are either unsubstituted or substituted by one or more substituents selected from halogen atoms and C₁₋₆ alkyl, C₁₋₆ haloalkyl, C₁₋₆ alkoxy, —(C₁₋₆ alkyl)ₙC(O)O(C₁₋₆ alkyl) (where n=0 or 1), —(C₁₋₆ alkyl)ₙOC(O)(C₁₋₆ alkyl) (where n=0 or 1), C₁₋₆ alkylthiol, —N(R_{N})₂ (wherein each R_{N} independently represents a hydrogen atom or a C₁₋₆ alkyl group), —CN, —S(O)₂NH₂, nitro and sulfonic acid groups; or
(b) independently represent a C₁₋₆ alkyl group, a C₂₋₆ alkenyl group or a C₂₋₆ alkynyl group, and are unsubstituted or substituted by one or more substituents selected from halogen atoms, sulfonic acid groups and hydroxy groups;
(Y):
—R_{C}—R₆—N(R_{8A})(R_{8B}) together form a group of formula (VI) 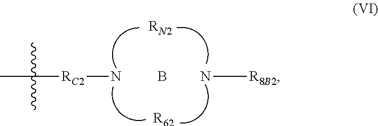
in which
R_{C2} represents —C(O)—, —S(O)₂—, —N(R_{N})—C(O)—, —N(R_{N})—C(S)—, —C(CF₃)— or —OC(O)—, Ring B is a 5-10 membered heterocyclylene ring containing both the nitrogen atom bound to R_{C2} and the nitrogen atom bound to R_{8B2}, and in which R_{N2} and R₆₂ are each alkylene groups, and
R_{8B2} is a C₁₋₆ alkyl group, a C₂₋₆ alkenyl group or a C₂₋₆ alkynyl group, and is unsubstituted or substituted by one or more substituents selected from halogen atoms, sulfonic acid groups, and hydroxy groups.

2. The cyclosporine analogue according to claim 1, wherein the moiety —R_{C}—R₆—N(R_{8A})(R_{8B}) is defined according to (X) and wherein R_{8A} and R_{8B} either:
(a) together with the nitrogen atom to which they are attached, form a 5- to 10-membered heteroaryl group or 5- to 10-membered heterocyclyl group, wherein said heteroaryl group and said heterocyclyl group are either unsubstituted or substituted by one or more substituents selected from halogen atoms and C₁₋₆ alkyl, C₁₋₆ haloalkyl, C₁₋₆ alkoxy, —(C₁₋₆ alkyl)ₙC(O)O(C₁₋₆ alkyl) (where n=0 or 1), —(C₁₋₆ alkyl)ₙOC(O)(C₁₋₆ alkyl) (where n=0 or 1), C₁₋₆ alkylthiol, —N(R_{N})₂ (wherein each R_{N} independently represents a hydrogen atom or a C₁₋₆ alkyl group), —CN, —S(O)₂NH₂, nitro and sulfonic acid groups; or
(b) independently represent a C₁₋₆ alkyl group, a C₂₋₆ alkenyl group or a C₂₋₆ alkynyl group, and are unsubstituted or substituted by one or more substituents selected from halogen atoms and sulfonic acid groups.

3. The cyclosporine analogue according to claim 1, wherein, in Ring A, said C₆₋₁₀ arylene group or 5- to 10-membered heteroarylene group is a naphthylene group, a 9- to 10-membered heteroarylene group or a phenylene group.

4. The cyclosporine analogue according to claim 3, wherein said C₆₋₁₀ arylene group or 5- to 10-membered heteroarylene group is a naphthylene group.

5. The cyclosporine analogue according to claim 3, wherein, said C₆₋₁₀ arylene group or 5- to 10-membered heteroarylene group is a 9- to 10-membered heteroarylene group.

6. (canceled)

7. The cyclosporine analogue according to claim 3, wherein said C₆₋₁₀ arylene group or 5- to 10-membered heteroarylene group is a phenylene group.

8. (canceled)

9. The cyclosporine analogue according to claim 1, wherein Ring A is unsubstituted or substituted by one or two of said substituents.

10. (canceled)

11. The cyclosporine analogue according to claim 1, wherein the moiety —R_{C}—R₆—N(R_{8A})(R_{8B}) is defined according to (X) and wherein R_{C} represents a group selected from the group consisting of —C(O)O—, —C(O) NH—, —S(O)₂NH—, —NH—C(O)—NH—, —NH—C(S)—NH—, —NH—C(O)—O— and

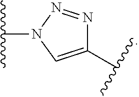 [SMILES: *c1cn(*)nn1]

12. (canceled)

13. The cyclosporine analogue according to claim 1, wherein the moiety —R_{C}—R₆—N(R_{8A})(R_{8B}) is defined according to (X) and wherein R₆ represents a C₂₋₄ alkylene group or a C₂₋₄ alkenylene group, and preferably wherein: (a) R₆ is ethylene, —(CH₂)₂—; or (b) R₆ is —(CH₂)₄—.

14. The cyclosporine analogue according to claim 1, wherein the moiety —R_{C}—R₆—N(R_{8A})(R_{8B}) is defined according to (X) and wherein R_{8A} and R_{8B} either:
(a) together with the nitrogen atom to which they are attached, form a 5- to 6-membered heteroaryl group or 5- to 6-membered heterocyclyl group; or
(b) independently represent a C₁₋₄ alkyl group or a C₂₋₄ alkenyl group.

15. The cyclosporine analogue according to claim 14, wherein R_{8A} and R_{8B}, together with the nitrogen atom to which they are attached, form an imidazolyl group, preferably of the formula

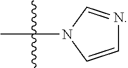 [SMILES: *n1ccnc1]

16. The cyclosporine analogue according to claim 1, wherein:
R₁ represents hydrogen;
R₂ represents 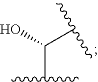
R₃ represents ethyl;
R₄ represents methyl;
R₅ represents —CH₂CH(CH₃)₂; and
R₇ represents hydrogen.

17. The cyclosporine analogue according to claim 1, which has at least one and preferably both of (a) and (b):
(a) the cyclosporine analogue has a lower binding affinity than cyclosporine A (CsA) to cyclophilin A (CypA);
(b) the cyclosporine analogue is an IFITM3 inhibitor.

18. The cyclosporine analogue according to claim 1, which is a compound selected from the following compounds, or is a pharmaceutically acceptable salt thereof:

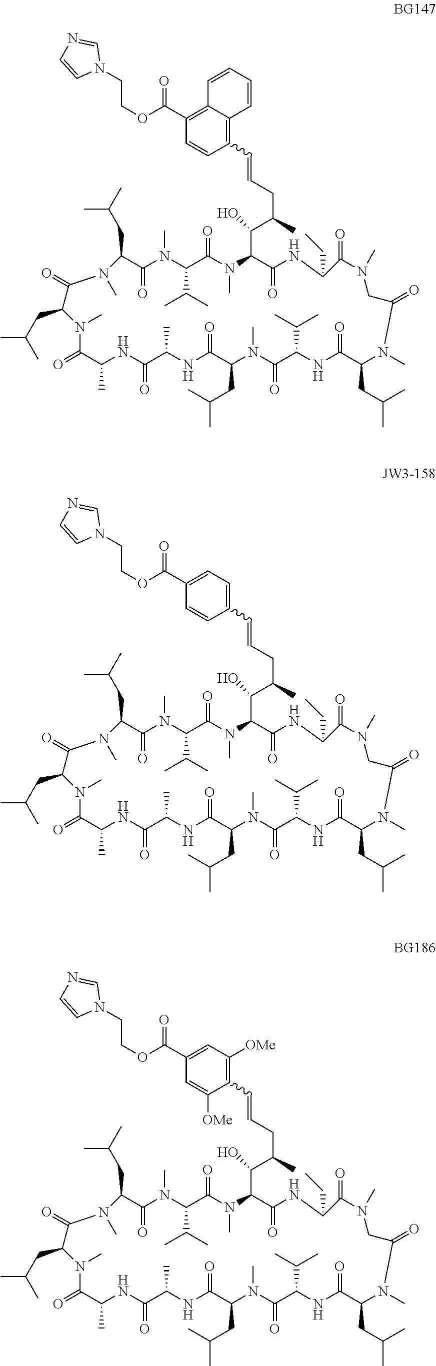

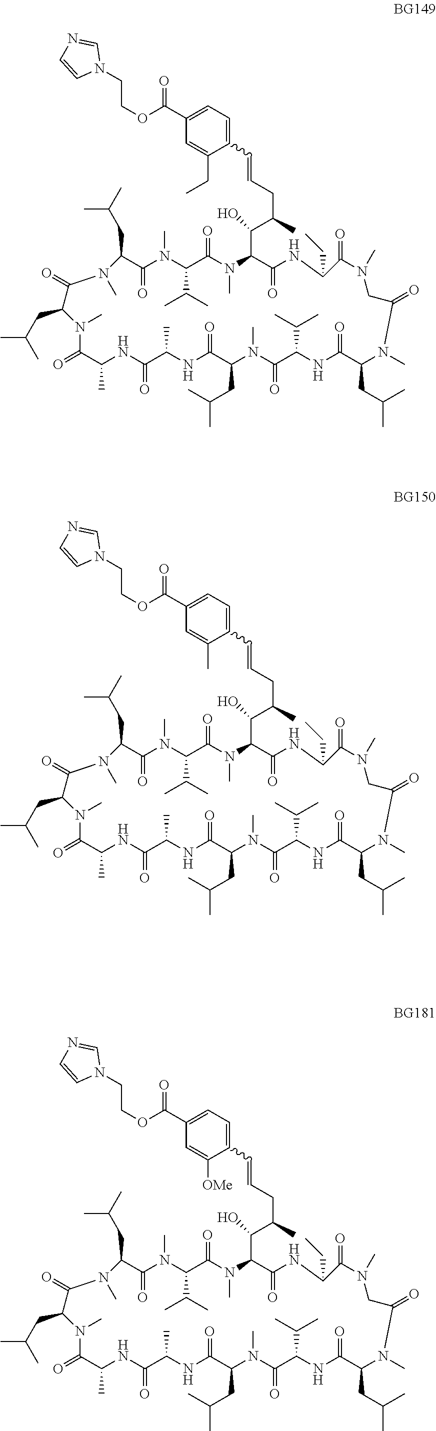

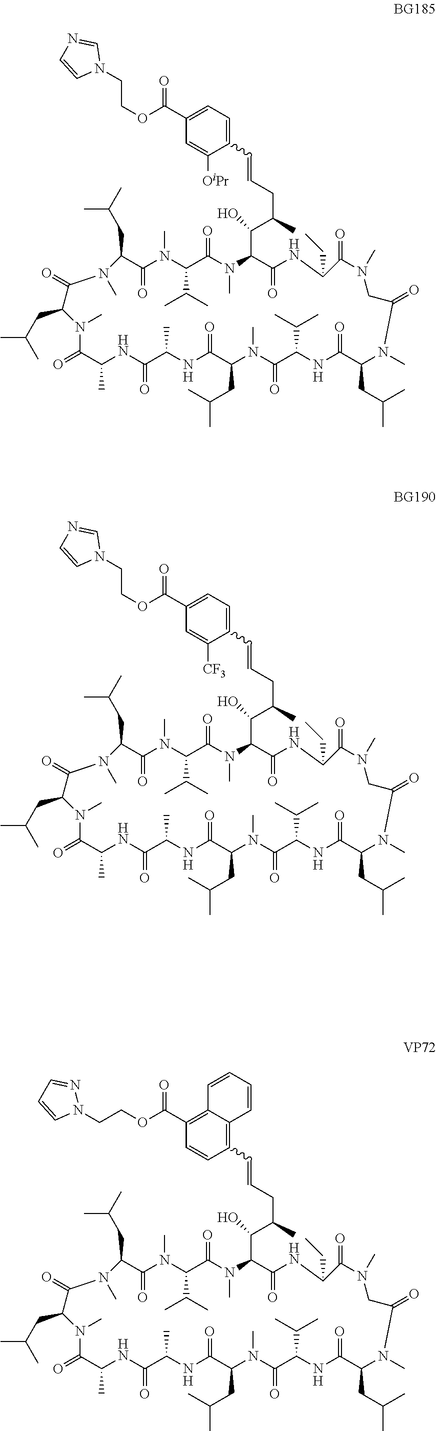

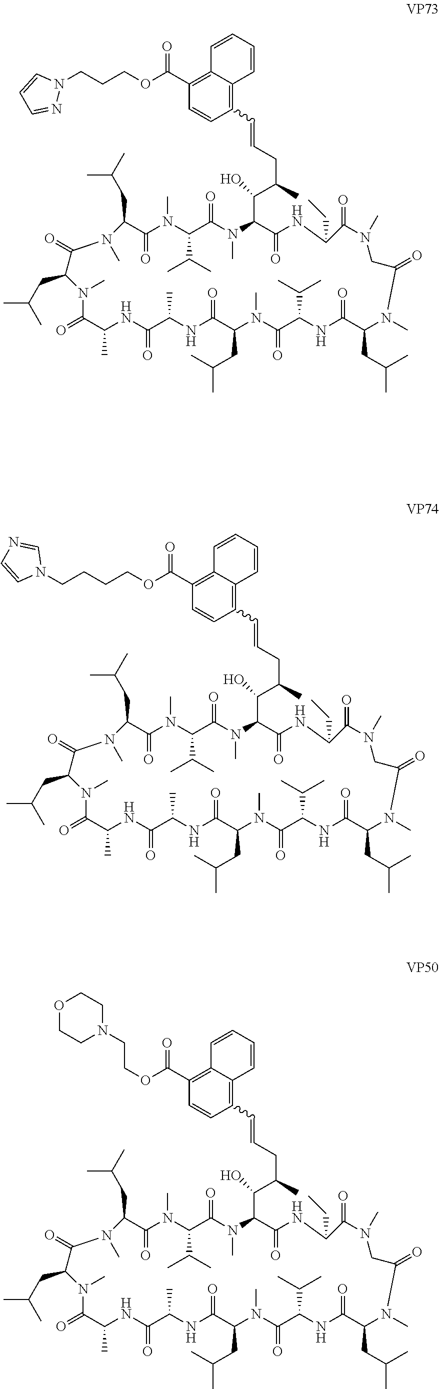

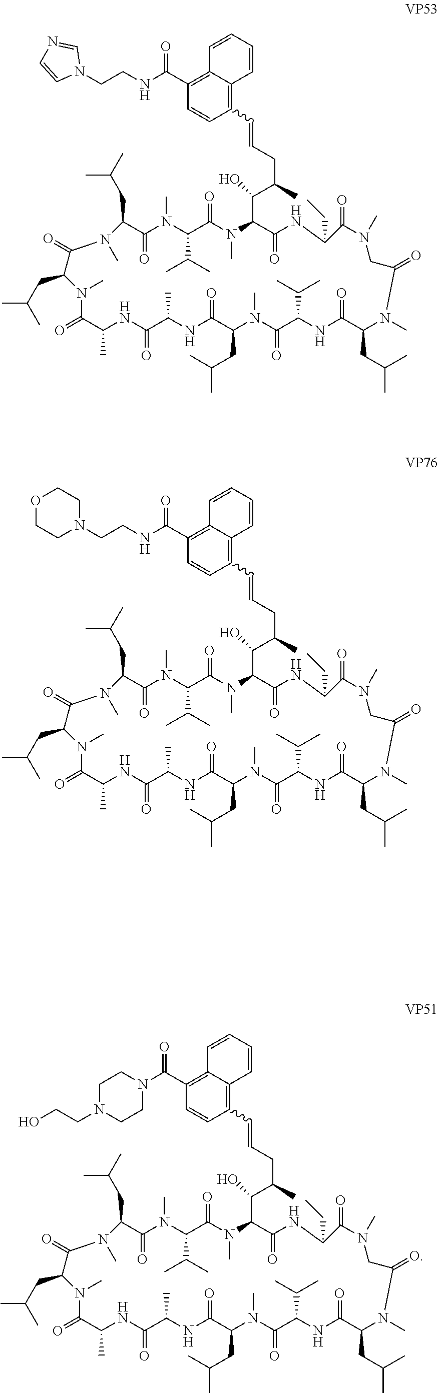

19. (canceled)

20. A method of transducing a population of mammalian cells, preferably wherein the mammalian cells are human cells, and more preferably wherein the population of mammalian cells is selected from human haematopoietic stem and/or progenitor cells, induced human haematopoietic stem and/or progenitor cells, and/or cells differentiated from the human haematopoietic stem and/or progenitor cells or induced human haematopoietic stem and/or progenitor cells such as cells selected from one or more of a common myeloid progenitor, a megakaryocyte, an erythroblast, a mast cell, a myeloblast, a basophil, a neutrophil, an eosinophil, a monocyte, a common lymphoid progenitor, a natural killer cell, a T cell such as an α/β T cell, a γδ T cell or a regulatory T cell, a B cell and a plasma cell, comprising the steps of:
a) contacting the population of cells with the cyclosporine analogue according to claim 1; and
b) transducing the population of cells with a vector derived from HIV-1, HIV-2, FIV, BIV, EIAV, CAEV or visna lentivirus;
the method optionally having at least one of the following further features (i) to (iv):
(i) steps (a) and (b) are carried out ex vivo or in vitro;
(ii) the percentage of mammalian cells, human cells, haematopoietic stem and/or progenitor cells, induced haematopoietic stem and/or progenitor cells, and/or cells differentiated from the haematopoietic stem and/or progenitor cells or induced haematopoietic stem and/or progenitor cells such as cells selected from one or more of a common myeloid progenitor, a megakaryocyte, an erythroblast, a mast cell, a myeloblast, a basophil, a neutrophil, an eosinophil, a monocyte, a common lymphoid progenitor, a natural killer cell, a T cell such as an α/β T cell, a γδ T cell or a regulatory T cell, a B cell and a plasma cell transduced by the vector is increased and/or the vector copy number per cell is increased;
(iii) the population of mammalian cells, human cells, haematopoietic stem and/or progenitor cells, induced haematopoietic stem and/or progenitor cells, and/or cells differentiated from the haematopoietic stem and/or progenitor cells or induced haematopoietic stem and/or progenitor cells such as cells selected from one or more of a common myeloid progenitor, a megakaryocyte, an erythroblast, a mast cell, a myeloblast, a basophil, a neutrophil, an eosinophil, a monocyte, a common lymphoid progenitor, a natural killer cell, a T cell such as an α/β T cell, a γδ T cell or a regulatory T cell, a B cell and a plasma cell is or has been obtained from mobilised peripheral blood, bone marrow or umbilical cord blood; and
(iv) the method includes a further step of enriching the population for mammalian cells, human cell, haematopoietic stem and/or progenitor cells, induced haematopoietic stem and/or progenitor cells, and/or cells differentiated from the haematopoietic stem and/or progenitor cells or induced haematopoietic stem and/or progenitor cells such as cells selected from one or more of a common myeloid progenitor, a megakaryocyte, an erythroblast, a mast cell, a myeloblast, a basophil, a neutrophil, an eosinophil, a monocyte, a common lymphoid progenitor, a natural killer cell, a T cell such as an α/β T cell, a γδ T cell or a regulatory T cell, a B cell and a plasma cell.

21. A method of gene therapy comprising the steps of:
a) transducing a population of mammalian cells, preferably wherein the mammalian cells are human cells, and more preferably wherein the population of mammalian cells is selected from human haematopoietic stem and/or progenitor cells, induced human haematopoietic stem and/or progenitor cells, and/or cells differentiated from the human haematopoietic stem and/or progenitor cells or induced human haematopoietic stem and/or progenitor cells such as cells selected from one or more of a common myeloid progenitor, a megakaryocyte, an erythroblast, a mast cell, a myeloblast, a basophil, a neutrophil, an eosinophil, a monocyte, a common lymphoid progenitor, a natural killer cell, a T cell such as an α/β T cell, a γδ T cell or a regulatory T cell, a B cell and a plasma cell according to the method of claim 20; and
b) administering the transduced cells to a subject;
and optionally wherein the transduced cells are administered to a subject as part of an autologous stem cell transplant procedure or an allogeneic stem cell transplant procedure.

22. A population of mammalian cells, human cells, human haematopoietic stem and/or progenitor cells, induced human haematopoietic stem and/or progenitor cells, and/or cells differentiated from the human haematopoietic stem and/or progenitor cells or induced human haematopoietic stem and/or progenitor cells such as cells selected from one or more of a common myeloid progenitor, a megakaryocyte, an erythroblast, a mast cell, a myeloblast, a basophil, a neutrophil, an eosinophil, a monocyte, a common lymphoid progenitor, a natural killer cell, a T cell such as an α/β T cell, a γδ T cell or a regulatory T cell, a B cell and a plasma cell, prepared according to the method of claim 2018.

23. A pharmaceutical composition comprising the population of mammalian cells, human cells, haematopoietic stem and/or progenitor cells, induced haematopoietic stem and/or progenitor cells, and/or cells differentiated from the haematopoietic stem and/or progenitor cells or induced haematopoietic stem and/or progenitor cells such as cells selected from one or more of a common myeloid progenitor, a megakaryocyte, an erythroblast, a mast cell, a myeloblast, a basophil, a neutrophil, an eosinophil, a monocyte, a common lymphoid progenitor, a natural killer cell, a T cell such as an α/β T cell, a γδ T cell or a regulatory T cell, a B cell and a plasma cell, according to claim 22.

24.-26. (canceled)

27. A method of treating a pathological condition associated with IFITM3 expression in a patient in need thereof, which comprises administering to the patient an effective amount of a cyclosporine analogue according to claim 1, preferably wherein the pathological condition is selected from the group consisting of a viral infection and Alzheimer's disease, more preferably wherein the pathological condition is a corona virus, and more preferably still wherein the pathological condition is COVID-19.

28. A compound of formula

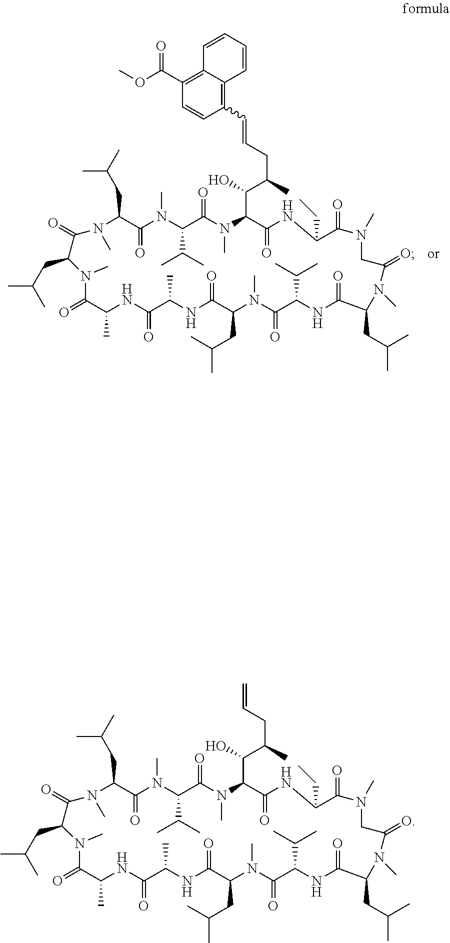

29. (canceled)
